(19) United States
(12) Patent Application Publication
(10) Pub. No.: US 2025/0327028 A1
(43) Pub. Date: Oct. 23, 2025

(54) ENGINEERED PLATELETS AS TARGETED PROTEIN DEGRADERS

(71) Applicant: Wisconsin Alumni Research Foundation, Madison, WI (US)

(72) Inventors: Quanyin Hu, Waunakee, WI (US); Yu Chen, Madison, WI (US)

(73) Assignee: Wisconsin Alumni Research Foundation, Madison, WI (US)

(21) Appl. No.: 18/639,579

(22) Filed: Apr. 18, 2024

(51) Int. Cl. C12N 5/078; A61K 35/19; A61K 38/00; A61P 35/00; C07K 14/47

(52) U.S. Cl. CPC C12N 5/0644; A61K 35/19; A61P 35/00; C07K 14/4702; A61K 38/00; C12N 2510/00

(57) ABSTRACT

Described herein are engineered platelets including a platelet cell or platelet-derived microparticle loaded with an HSP90 anchoring chimera including a protein of interest (POI) ligand covalently linked to a heat shock protein 90 (HSP90). The POI can be an intracellular or extracellular POI. Also described are methods of making the engineered platelets, and methods of degrading intracellular and extracellular POIs at a disease site.

### FIELD OF THE DISCLOSURE

The present disclosure is related to a targeted protein degradation platform that combines ubiquitin-proteasome system (UPS)- and endosome-lysosome pathway-mediated targeted protein degradation into endogenous platelets, which guide the effector protein pre-labeled with the ligand for the protein of interest (POI) to the diseased site for degradation of the intracellular or extracellular POI.

### BACKGROUND

Emerging targeted protein degradation (TPD) strategies employ a chimeric molecule to simultaneously capture an effector protein and a protein of interest (POI) to form a ternary complex, thereby repurposing cellular proteolytic machinery to degrade the POI. Various bioactive modules that can bind to the POIs or the effectors, including small molecules, peptides, and oligonucleotides, have been explored for developing degraders to eliminate intracellular disease-causing proteins by hijacking the ubiquitin-proteasome system (UPS) or autophagy-lysosomal pathway. Other rationally designed chimeras, commonly constructed by antibodies or small-molecule ligands, could degrade the extracellular targets through the endocytosis-lysosomal machinery. In vivo applications of chimera-mediated TPD technologies are often plagued with intrinsic limitations hidden in their unique molecular and pharmacological properties.

Heterobifunctional chimeric molecules that bridge two binders by a linker frequently suffer from unfavorable drug-like properties, such as solubility, permeability, and biocompatibility. Coupled with non-specific biodistribution, the chimeric structures may suffer from limited accumulation in the lesion and increased risks of off-target or off-tissue on-target side effects associated with having two different functional ligands. Most chimeras, especially those containing other modular entities, have not progressed beyond the preclinical stage. More importantly, even when accumulated at the disease site, the chimera-induced proximity between the effector and POI, the pharmacological basis of both UPS- and lysosome-mediated TPD, requires good spatiotemporal cooperativity among the chimera and these two proteins. To degrade specific dysregulated POI by leveraging the formation of the ternary complex, the balance of effector abundance and chimera dose within the lesion raises additional challenges in chimera development and delivery course than the conventional therapies (e.g., small molecule inhibitor and antibody therapy). Collectively, these shortcomings of the chimeric protein degraders rooted in their unique structures and mechanisms of action impose barriers to efficient and safe in vivo TPD.

What is needed are novel strategies for in vivo TPD.

### BRIEF SUMMARY

In an aspect, an engineered platelet comprises a platelet cell or platelet-derived microparticle loaded with an HSP90 chimera comprising a protein of interest (POI) ligand covalently linked to a heat shock protein 90 (HSP90).

In an aspect, a method of making an engineered platelet comprises covalently linking a ligand for a POI to an HSP90 to form an HSP90 chimera, and incubating a suspension of platelet cells or platelet-derived microparticles with the HSP90 chimera to load the HSP-90 chimera in the platelet cells or platelet-derived microparticles and form the engineered platelets.

In another aspect, a method of degrading an intracellular POI via a ubiquitin-protease system comprises contacting a disease site with the engineered platelet described herein, wherein the POI ligand binds the intracellular POI, and wherein the engineered platelet transfers the HSP90 chimera to cells at the disease site, thereby binding to and degrading the intracellular POI.

In yet a further aspect, a method of degrading an extracellular POI via a lysosome-associated pathway comprises contacting a disease site with the engineered platelet described herein, wherein the POI ligand binds the extracellular POI, and wherein the engineered platelet releases the HSP90 anchoring chimera to the extracellular space at the disease site, thereby binding to the extracellular POI and guiding it to the lysosome for degradation.

### BRIEF DESCRIPTION OF THE DRAWINGS

FIG. 1 is a schematic illustration of platelet-based protein degraders with covalently labeling HSP90 realizing TPD for intracellular or extracellular targets in vivo. Engineered platelets with POI ligand-tethered HSP90 (DePLT) were generated through ligand-directed covalent labeling, which could selectively reach the post-surgical area and undergo activation due to tropism towards hemorrhage. According to the distinct POI ligands tethered, DePLT could transfer labeled HSP90 to cancer cells through PMPs and induce degradation for intracellular POI via UPS, or DePLT could release the pre-labeled free HSP90 to redirect extracellular POI to lysosome for proteolysis.

FIGS. 2A-N show a BRD4-targeting HSP90-anchoring chimera (designated iHAC) degrades the intracellular POI by covalent and non-covalent HSP90 binding. (2A) Chemical structure of biotin-labeled HSP90-anchoring chimera (designated HAC-B). (2B) Immunoblot analysis of the biotin-labeled protein (detected by HRP-streptavidin) in 4T1 cells treated with HAC-B at various concentrations for 2 h. (2C) Gel electrophoresis and immunoblot analysis of the biotin-labeled protein immunoprecipitated by streptavidin beads in the lysate of 4T1 cells treated with HAC-B at 10 μM for 2 h. The arrow indicates the possible HSP90 band. (2D) Mass spectrometry analysis of the biotin-labeled protein immunoprecipitated by streptavidin beads in the lysate of 4T1 cells treated with HAC-B at 10 M for 4 h. Proteins with more than 2 unique peptides were shown. The number/percentage indicates the amount of identified peptides and the coverage of identified peptides on the relevant protein. (2E) Chemical structure of iHAC-B. (2F and 2G) Immunoblot analysis of BRD4 degradation in 4T1 (F) and WT MDA-MB-231 (G) cells after iHAC treatment at various concentrations for 24 h. (2H) Immunoblot analysis of HSP90α expression in WT MDA-MB-231 and HSP90α-knock-out MDA-MB-231 cells and BRD4 degradation in HSP90α-knock-out MDA-MB-231 cells after iHAC treatment at various concentrations for 24 h. (2I) Chemical structure of HAI. (2J) Immunoblot analysis of BRD4 degradation in 4T1 cells after the treatments with iHAC at 10 μM for 12 h with or without 2-h pretreatments of HAI at various concentrations. (2K and 2L) Immunoblot analysis of BRD4 degradation in 4T1 (K) and WT MDA-MB-231 (L) cells after the treatments with iHAC, iHACepi, the combinations of iHAC plus Epox, JQ1 plus PU-H71, HAC-B plus JQ1 at 0.16 μM and 10 M for 12 h, respectively. (2M) Immunoblot analysis of BRD4 precipitated by the anti-HSP90 antibody in the lysate of 4T1 cells treated with iHAC at 10 μM for 2 h. (2N) Schematic illustration of iHAC-induced degradation of the intracellular protein by covalently and non-covalently HSP90 binding.

FIGS. 3A-M show activated iDePLT packages BRD4 ligand-labeled HSP90 into PMPs for BRD4 degradation in cancer cells. (3A) Visualization of membrane fusion between PMPs and 4T1 cells by staining PMPs with Rhodamine-labeled wheat germ agglutinin (WGA) and 4T1 cells with DiO. Hoechst 33342 was used to indicate the nucleus. Scale bar, 10 μm. (3B) Detection of HSP90 encapsulated in PMPs released from activated platelets. (3C and 3D) Immunoblot analysis of the biotin-labeled HSP90 precipitated by streptavidin (SA) beads in the lysate of platelets treated with HAC-B at 10 μM for various time points (3C) or at various concentrations for 2 h (3D). (3E) Biotin-labeled HSP90 in platelets treated with HAC-B at various concentrations for 2 h. (3F) Release of biotin-labeled HSP90 from resting PLT-B and activated PLT-B at different time points. Thrombin was used to trigger the activation of PLT-B. (3G) Distribution of biotin-labeled HSP90 released from PLT-B in 4T1 cells as detected by streptavidin-FITC. Rhodamine (red)-labeled WGA and Hoechst 33342 were used to indicate the cell membrane and nucleus, respectively. Scale bar, 25 μm. (3H) Immunoblot analysis of BRD4 degradation in 4T1 cells after coincubation with a total of 2×10⁸ platelets for 24 h. The amounts indicate the numbers of iDePLT mixed with nPLT. (3I) Immunoblot analysis of BRD4 degradation in 4T1 cells after the treatments with resting or activated platelets. (3J) Immunoblot analysis of BRD4 degradation in 4T1 cells after the treatments with 1×10⁸ iDePLTs for 12 h with or without 2-h pretreatments of HAI at various concentrations. (3K) Immunoblot analysis of BRD4 degradation in 4T1 cells after the treatments with various PMPs released from their corresponding parent platelets. Epox was used as a proteasome inhibitor. (3L) Immunoblot analysis of BRD4 precipitated by the anti-HSP90 antibody in the lysate of 4T1 cells treated with the DePMP released from 1×10⁸ iDePLTs for 6 h. (3M) Volcano plots of the Log₂ (fold change) versus the −Log 10 (P value) presenting the abundance changes of global proteins in 4T1 cells after coincubation with nPLT versus iDePLT for 12 h. The plot indicates BRD4.

FIGS. 4A-I show iDePLT inhibits tumor recurrence and metastasis in post-surgery mice with primary breast cancer. (4A) Operation schedule of the treatments and evaluations in post-surgery mice with primary 4T1-Luc tumors. (4B) Quantified bioluminescence intensities of the primary breast sites in post-surgery mice from different treatment groups. (4C) Quantified bioluminescence intensities of the lung metastasis areas in post-surgery mice from different treatment groups. The insert images show metastatic nodules in the isolated lung tissues after treatments. (4D) Survival curves of post-surgery mice from the indicated treatment groups. Data are analyzed with Log-rank (Mantel-Cox) test. (4E) Body weight changes of post-surgery mice during the treatments. Data are analyzed with two-way ANOVA. For (D) and (E), all data represent seven independent experiments, (4F) Blood biochemical analysis of post-surgery mice in saline- or iDePLT-treated groups. All data represent three independent experiments and were analyzed by unpaired Student's t-test. ALT,alanine transaminase; AST, aspartate transferase; BUN, blood urea nitrogen. (4G) Experimental design of post-surgery NSG mice with orthotopic PDX breast cancer. (4H and I) Weights (H) and image (I) of relapsed tumors from post-surgery NSG mice with primary breast cancer PDXs in different treatment groups. Data are analyzed with one-way ANOVA. Scale bar, 1 cm. For (4D), (4E), (4F), and (4H), ns indicates no significance, *P<0.05, **P<0.01.

FIGS. 5A-N show eDePLT releases PD-L1 ligand-labeled extracellular HSP90 to degrade PD-L1 on cancer cells by repurposing lysosome machinery. (5A) Detection of the free extracellular HSP90 released from activated platelets. (5B) Internalization of streptavidin-FITC in HAC-B-pretreated 4T1 cells pretreated with HAC-B at 5 μM for 2 h. Scale bar, 25 μm. (5C and 5D) Internalization of NA-647 in WT MDA-MB-231 cells pretreated with HAC-B at 10 μM for various time points (5C) or at the indicated concentrations (5D) for 4 h and then incubated in neutravidin (NA)-647 (0.5 μM)-containing medium for 1 h. (5E) Internalization of NA-647 in WT MDA-MB-231 cells after the treatments with HAC-B at 5 μM for 4 h with or without 2-h pretreatments of HAI at various concentrations. (5F) Internalization of NA-647 in WT MDA-MB-231 cells after the pretreatments with HAC-B, the combinations of HAC-B plus PU-H71, IM-6 plus free biotin, and PU-H71 plus free biotin at 5 μM for 4 h. (5G) Colocalization between internalized NA-647 and lysosome marker (LAMP1) in 4T1 cells pretreated with HAC-B at 5 μM for 4 h and incubated in NA-647 (0.5 μM)-containing medium for 1 h. Scale bar, 25 μm. (5H) Chemical structure of HAC targeting PD-L1 (designated eHAC). (5I) Immunoblot analysis of PD-L1 degradation in 4T1 cells treated with eHAC at various concentrations for 8 h incubated in the conditioned medium (CM). (5J) PD-L1 distribution in WT MDA-MB-231 cells treated with eHAC or the combination of PU-H71 plus BMS-1166 at 5 μM for 2 h. Scale bar, 25 μm. (5K) Immunoblot analysis of PD-L1 degradation in 4T1 cells after the treatments with eHAC, the combinations of PU-H71 plus BMS-1166, eHAC plus lysosome degradation inhibitor (Leupeptin), and HAC-B plus BMS-1166 at 5 μM for 8 h. (5L) Immunoblot analysis of HSP90α and PD-L1 expression in PEA-10 cells treated with eHAC at various concentrations for 8 h incubated in CM. (5M) Immunoblot analysis of PD-L1 degradation in 4T1 cells after coincubation with a total of 2×10⁸ platelets for 24 h. The amounts indicate the numbers of eDePLT mixed with nPLT. (5N) Immunoblot analysis of PD-L1 degradation in 4T1 cells after the treatments with resting or activated platelets. (5O) Immunoblot analysis of PD-L1 degradation in 4T1 cells after the treatments with 1×10⁸ eDePLTs for 8 h with or without 2-h pretreatments of HAI at various concentrations. For (B), (G), and (J), Hoechst 33342 was used to indicate the cell nucleus.

FIGS. 6A-G show eDePLT degrades PD-L1 and elicits potent antitumor immune response against TNBC recurrence and metastasis in a post-surgical mouse model. (6A) Operation schedule of the treatments and evaluations in post-surgical mice with primary 4T1-Luc tumors. (6B) Quantified bioluminescence intensities of the primary breast sites in post-surgery mice from different treatment groups. (6C) Quantified bioluminescence intensities of the lung metastasis areas in post-surgery mice from different treatment groups. The insert images show metastatic nodules in the isolated lung tissues after treatments. (6D) Plot illustrating flow cytometry analysis of CD8⁺ T cells out of the total CD3⁺ cell population. (6E) Percentages of CD8⁺ T cells out of the total CD3⁺ cell population within the tumor tissues after the indicated treatments. (6F) IFN-γ and TNF-α levels within the tumor tissues after the indicated treatments detected by the ELISA assay. (6G) Survival curves of post-surgery mice from the indicated treatment groups. Data are analyzed with Log-rank (Mantel-Cox) test. For (E) and (F), all data represent five independent experiments and are presented as mean±SD (n=5), *P<0.05, **P<0.01, ***P<0.01.

The above-described and other features will be appreciated and understood by those skilled in the art from the following detailed description, drawings, and appended claims.

### DETAILED DESCRIPTION

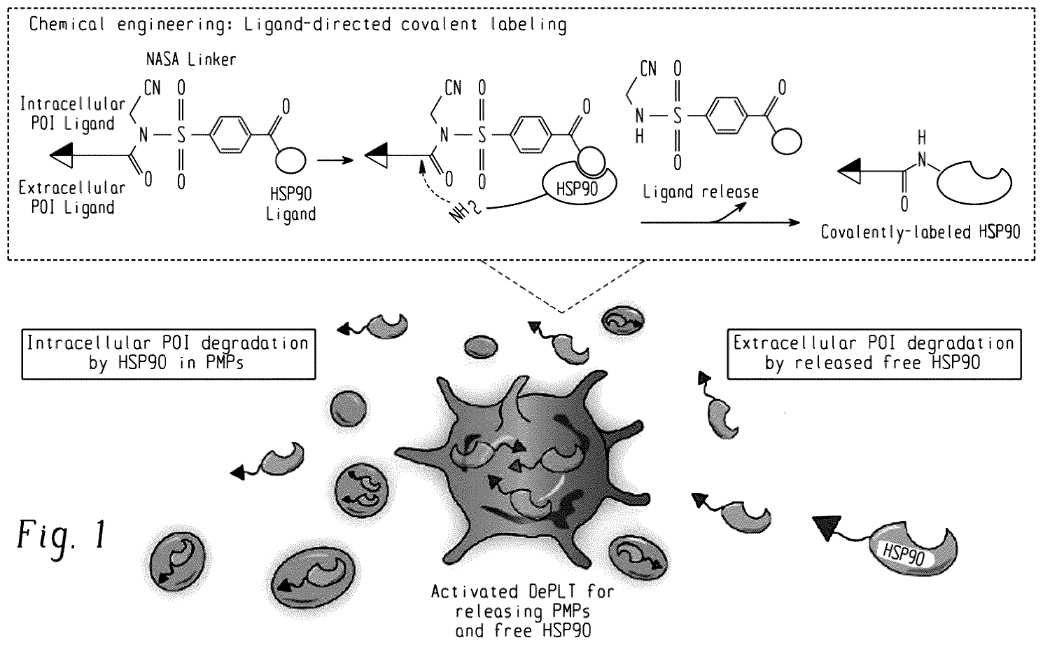

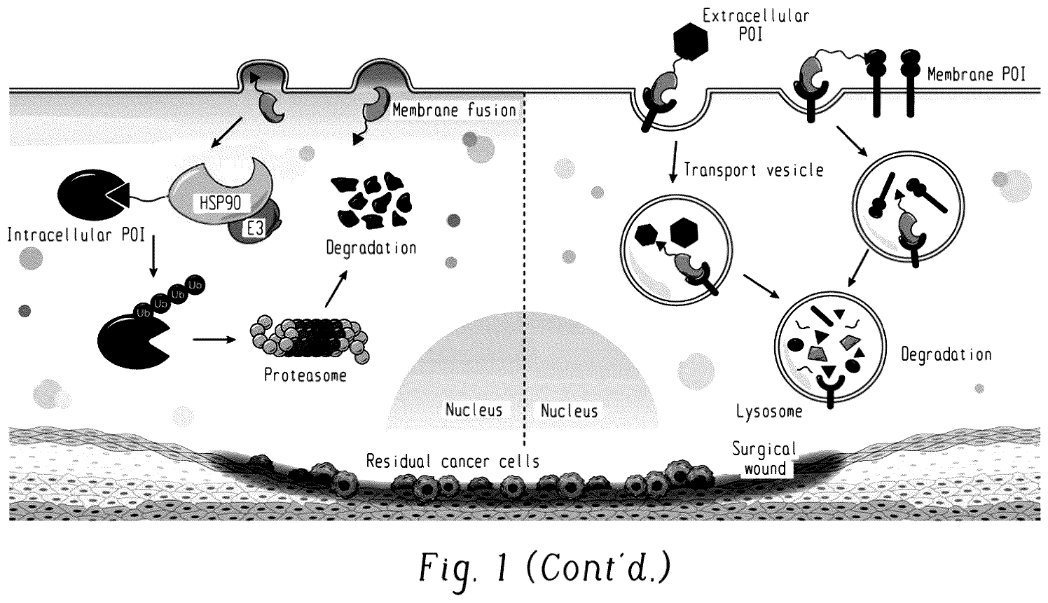

Described herein is a cell-based protein degradation platform established by grafting UPS- and lysosome-mediated TPD concepts into endogenous platelets to address challenges in in vivo applications of chimeric molecules (FIG. 1). Specifically, the POI ligand was covalently tethered to heat shock protein 90 (HSP90) within platelets through a facile chemical engineering method. These proteolytic platelets, termed DePLT, could inherently and selectively accumulate at wound-associated disease sites and then potently degrade the POI by repurposing the critical role of molecular chaperones in protein processing. Based on the distinct POI ligands tethered within the activated DePLT, the pre-labeled HSP90 could be packaged into platelet-derived microparticles (PMPs) and transferred to the targeted cells through membrane fusion, wherein the labeled HSP90 was able to capture the intracellular POI and trigger the USP-mediated degradation; alternatively, the POI ligand-tethered free HSP90 could be released to surrounding environment from activated DePLT and bind to the extracellular POI, thereby guiding it along the endosome-lysosome pathway for degradation. It is demonstrated herein that DePLT efficiently suppressed tumor recurrence and metastasis in post-surgical triple-negative breast cancer (TNBC)-bearing mouse models by targeting representative intracellular POI, bromodomain-containing protein 4 (BRD4), and substantially enhanced the anticancer immune response in vivo through the degradation of immune-associated extracellular POI, programmed death-ligand 1 (PD-L1). Collectively, to facilitate in vivo applications of TPD technologies, a platelet-templated TPD strategy is expected to overcome the limitations in the commonly used chimera concepts.

In an aspect, an engineered platelet comprises a platelet cell or platelet-derived microparticle loaded with an HSP90 chimera comprising a protein of interest (POI) ligand covalently linked to a heat shock protein 90 (HSP90, also called HSPC).

As used herein, a platelet cell can be prepared by isolating platelets from whole blood by centrifuging whole blood and separating the platelet-rich plasma. The platelets can then be separated from the plasma by centrifugation.

Platelet-derived microparticles (PMP) are nano-size fragments (100-1000 nm) released from platelets under certain conditions, such as thrombin treatment or vortexing.

In an aspect, the platelet cell, or platelet-derived microparticle is of human origin. Advantageously, platelet cells, platelet-derived microparticles, and platelet membranes can comprise platelet proteins capable of interacting with cells such as cancer cells.

As used herein, the term HSP90 includes both isoforms of HSP90: HSP90-alpha (HSP90α, also known as HSPC2, HSPAA2, HSPCA, and HSPCAL3) and HSP90-beta (HSP90bβ, also known as HSPC3, HSPAB1, and HSPCB).

In an aspect, the POI is an intracellular POI, which can be selected from the following table:

| | |
|---|---|
| ESR1 | Estrogen receptor |
| AR | Androgen receptor |
| BTK | Tyrosine-protein kinase BTK |
| IRAK4 | Interleukin-1 receptor-associated kinase 4 |
| EGFR | Epidermal growth factor receptor |
| MET | Hepatocyte growth factor receptor |
| KIT | Mast/stem cell growth factor receptor Kit |
| EPHA2 | Ephrin type-A receptor 2 |
| PDE4D | cAMP-specific 3′,5′-cyclic phosphodiesterase 4D |
| SRC | Proto-oncogene tyrosine-protein kinase Src |
| BRAF | Serine/threonine-protein kinase B-raf |
| FGFR2 | Fibroblast growth factor receptor 2 |
| FGFR1 | Fibroblast growth factor receptor 1 |
| LYN | Tyrosine-protein kinase Lyn |
| ITK | Tyrosine-protein kinase ITK/TSK |
| PARP1 | Poly [ADP-ribose] polymerase 1 |
| HDAC2 | Histone deacetylase 2 |
| HDAC3 | Histone deacetylase 3 |
| JAK1 | Tyrosine-protein kinase JAK1 |
| BCL2 | Apoptosis regulator Bcl-2 |
| HDAC6 | Histone deacetylase 6 |
| CRBN | Protein cereblon |
| EPHB2 | Ephrin type-B receptor 2 |
| BLK | Tyrosine-protein kinase Blk |
| HDAC1 | Histone deacetylase 1 |
| IGF1R | Insulin-like growth factor 1 receptor |
| TGFBR1 | TGF-beta receptor type-1 |
| AKT2 | RAC-beta serine/threonine-protein kinase |
| AKT1 | RAC-alpha serine/threonine-protein kinase |
| PTK2 | Focal adhesion kinase 1 |
| MAPK1 | Mitogen-activated protein kinase 1 |
| MAPK14 | Mitogen-activated protein kinase 14 |
| CDK9 | Cyclin-dependent kinase 9 |
| MCL1 | Induced myeloid leukemia cell differentiation protein Mcl-1 |
| BRD4 | Bromodomain-containing protein 4 |
| BRD3 | Bromodomain-containing protein 3 |
| CDK13 | Cyclin-dependent kinase 13 |
| BCL2L1 | Bcl-2-like protein 1 |
| CDK12 | Cyclin-dependent kinase 12 |
| CDK1 | Cyclin-dependent kinase 1 |
| AKT3 | RAC-gamma serine/threonine-protein kinase |
| CDK11B | Cyclin-dependent kinase 11B |
| PAK4 | Serine/threonine-protein kinase PAK 4 |
| MAPKAPK2 | MAP kinase-activated protein kinase 2 |
| TNK2 | Activated CDC42 kinase 1 |
| SIRT2 | NAD-dependent protein deacetylase sirtuin-2 |
| DAPK1 | Death-associated protein kinase 1 |
| ABL2 | Tyrosine-protein kinase ABL2 |
| PRKAA2 | 5′-AMP-activated protein kinase catalytic subunit alpha-2 |
| KAT2A | Histone acetyltransferase KAT2A |
| PBRM1 | Protein polybromo-1 |
| EIF2AK2 | Interferon-induced, double-stranded RNA-activated protein kinase |
| MAP3K7 | Mitogen-activated protein kinase kinase kinase 7 |
| MAPT | Microtubule-associated protein tau |
| RIPK1 | Receptor-interacting serine/threonine-protein kinase 1 |
| IRAK1 | Interleukin-1 receptor-associated kinase 1 |
| MAP4K1 | Mitogen-activated protein kinase kinase kinase kinase 1 |
| MARK4 | MAP/microtubule affinity-regulating kinase 4 |
| BRD9 | Bromodomain-containing protein 9 |
| RIPK2 | Receptor-interacting serine/threonine-protein kinase 2 |
| LIMK1 | LIM domain kinase 1 |
| STK38 | Serine/threonine-protein kinase 38 |
| TRIM24 | Transcription intermediary factor 1-alpha |
| SMARCA4 | Transcription activator BRG1 |
| PRKAA1 | 5′-AMP-activated protein kinase catalytic subunit alpha-1 |
| TBK1 | Serine/threonine-protein kinase TBK1 |
| KRAS | GTPase KRas |
| SMARCA2 | Probable global transcription activator SNF2L2 |
| PCNA | Proliferating cell nuclear antigen |
| BRD7 | Bromodomain-containing protein 7 |
| SUZ12 | Polycomb protein SUZ12 |
| IKZF1 | DNA-binding protein Ikaros |
| HTT | Huntingtin |
| SNCA | Alpha-synuclein |
| SLC9A1 | Sodium/hydrogen exchanger 1 |
| FER | Tyrosine-protein kinase Fer |
| MAP4K2 | Mitogen-activated protein kinase kinase kinase kinase 2 |
| DLG4 | Disks large homolog 4 |
| IKZF3 | Zinc finger protein Aiolos |
| SMAD3 | Mothers against decapentaplegic homolog 3 |
| PDE4A | cAMP-specific 3′,5′-cyclic phosphodiesterase 4A |
| HMGCR | 3-hydroxy-3-methylglutaryl-coenzyme A reductase |
| ABL1 | Tyrosine-protein kinase ABL1 |
| RARA | Retinoic acid receptor alpha |
| FLT3 | Receptor-type tyrosine-protein kinase FLT3 |
| RARG | Retinoic acid receptor gamma |
| FLT1 | Vascular endothelial growth factor receptor 1 |
| EZH2 | Histone-lysine N-methyltransferase EZH2 |
| EPHA1 | Ephrin type-A receptor 1 |
| AURKA | Aurora kinase A |
| CHEK1 | Serine/threonine-protein kinase Chk1 |
| WEE1 | Wee1-like protein kinase |
| PLK1 | Serine/threonine-protein kinase PLK1 |
| CDC7 | Cell division cycle 7-related protein kinase |
| AURKB | Aurora kinase B |
| PLK4 | Serine/threonine-protein kinase PLK4 |
| MDM2 | E3 ubiquitin-protein ligase Mdm2 |
| MAPK6 | Mitogen-activated protein kinase 6 |
| BUB1 | Mitotic checkpoint serine/threonine-protein kinase BUB1 |
| ESRRA | Steroid hormone receptor ERR1 |
| BCL6 | B-cell lymphoma 6 protein |
| MAPK12 | Mitogen-activated protein kinase 12 |
| CRABP2 | Cellular retinoic acid-binding protein 2 |
| HIPK1 | Homeodomain-interacting protein kinase 1 |
| ADRM1 | Proteasomal ubiquitin receptor ADRM1 |
| CDC20 | Cell division cycle protein 20 homolog |
| BUB1B | Mitotic checkpoint serine/threonine-protein kinase BUB1 beta |
| NTRK2 | BDNF/NT-3 growth factors receptor |
| NTRK1 | High affinity nerve growth factor receptor |
| CD274 | Programmed cell death 1 ligand 1 |
| NUAK1 | NUAK family SNF1-like kinase 1 |
| PDCD1 | Programmed cell death protein 1 |
| ERBB2 | Receptor tyrosine-protein kinase erbB-2 |
| EPHA3 | Ephrin type-A receptor 3 |
| PIK3CG | Phosphatidylinositol 4,5-bisphosphate 3-kinase catalytic subunit gamma isoform |
| MAP2K1 | Dual specificity mitogen-activated protein kinase kinase 1 |
| LCK | Tyrosine-protein kinase Lck |
| MAP2K2 | Dual specificity mitogen-activated protein kinase kinase 2 |
| PDE6D | Retinal rod rhodopsin-sensitive cGMP 3′,5′-cyclic phosphodiesterase subunit delta |
| JAK3 | Tyrosine-protein kinase JAK3 |
| GSK3B | Glycogen synthase kinase-3 beta |
| JAK2 | Tyrosine-protein kinase JAK2 |
| PRKCI | Protein kinase C iota type |
| FKBP1A | Peptidyl-prolyl cis-trans isomerase FKBP1A |
| DHODH | Dihydroorotate dehydrogenase |
| PIK3CA | Phosphatidylinositol 4,5-bisphosphate 3-kinase catalytic subunit alpha isoform |
| CDK6 | Cyclin-dependent kinase 6 |
| CDK4 | Cyclin-dependent kinase 4 |
| PDE4B | cAMP-specific 3′,5′-cyclic phosphodiesterase 4B |
| FYN | Tyrosine-protein kinase Fyn |
| TYK2 | Non-receptor tyrosine-protein kinase TYK2 |
| PDK1 | [Pyruvate dehydrogenase |
| PDK2 | [Pyruvate dehydrogenase |
| PDK3 | [Pyruvate dehydrogenase |
| YES1 | Tyrosine-protein kinase Yes |
| GSK3A | Glycogen synthase kinase-3 alpha |
| CDK16 | Cyclin-dependent kinase 16 |
| CSNK2A1 | Casein kinase II subunit alpha |
| CDK7 | Cyclin-dependent kinase 7 |
| PTK2B | Protein-tyrosine kinase 2-beta |
| MAPK10 | Mitogen-activated protein kinase 10 |
| MAPK9 | Mitogen-activated protein kinase 9 |
| MAPK8 | Mitogen-activated protein kinase 8 |
| CDK5 | Cyclin-dependent-like kinase 5 |
| METAP2 | Methionine aminopeptidase 2 |
| BRD2 | Bromodomain-containing protein 2 |
| MAPK3 | Mitogen-activated protein kinase 3 |
| TEC | Tyrosine-protein kinase Tec |
| CDK14 | Cyclin-dependent kinase 14 |
| CDK17 | Cyclin-dependent kinase 17 |
| MAP3K11 | Mitogen-activated protein kinase kinase kinase 11 |
| RPS6KB1 | Ribosomal protein S6 kinase beta-1 |
| CSK | Tyrosine-protein kinase CSK |
| MERTK | Tyrosine-protein kinase Mer |
| STK17B | Serine/threonine-protein kinase 17B |
| CSNK2A2 | Casein kinase II subunit alpha' |
| RPS6KA1 | Ribosomal protein S6 kinase alpha-1 |
| MAPK13 | Mitogen-activated protein kinase 13 |
| GAK | Cyclin-G-associated kinase |
| CLK1 | Dual specificity protein kinase CLK1 |
| STK4 | Serine/threonine-protein kinase 4 |
| EIF4E | Eukaryotic translation initiation factor 4E |
| STK10 | Serine/threonine-protein kinase 10 |
| LRRK2 | Leucine-rich repeat serine/threonine-protein kinase 2 |
| TAOK3 | Serine/threonine-protein kinase TAO3 |
| MARK2 | Serine/threonine-protein kinase MARK2 |
| CSNK1D | Casein kinase I isoform delta |
| AAK1 | AP2-associated protein kinase 1 |
| IRAK3 | Interleukin-1 receptor-associated kinase 3 |
| STAT3 | Signal transducer and activator of transcription 3 |
| CAMKK1 | Calcium/calmodulin-dependent protein kinase kinase 1 |
| EED | Polycomb protein EED |
| CSNK1A1 | Casein kinase I isoform alpha |
| NEK1 | Serine/threonine-protein kinase Nek1 |
| BMP2K | BMP-2-inducible protein kinase |
| MAPK7 | Mitogen-activated protein kinase 7 |
| ULK1 | Serine/threonine-protein kinase ULK1 |
| RPS6KA3 | Ribosomal protein S6 kinase alpha-3 |
| PTPN11 | Tyrosine-protein phosphatase non-receptor type 11 |
| LIMK2 | LIM domain kinase 2 |
| CSNK1E | Casein kinase I isoform epsilon |
| EIF2AK4 | eIF-2-alpha kinase GCN2 |
| MAP2K5 | Dual specificity mitogen-activated protein kinase kinase 5 |
| MAP4K3 | Mitogen-activated protein kinase kinase kinase kinase 3 |
| VHL | von Hippel-Lindau disease tumor suppressor |
| MARK3 | MAP/microtubule affinity-regulating kinase 3 |
| TAOK2 | Serine/threonine-protein kinase TAO2 |
| MAP4K5 | Mitogen-activated protein kinase kinase kinase kinase 5 |
| SNRK | SNF-related serine/threonine-protein kinase |
| EEF2K | Eukaryotic elongation factor 2 kinase |
| SGK3 | Serine/threonine-protein kinase Sgk3 |
| AHR | Aryl hydrocarbon receptor |
| NEK4 | Serine/threonine-protein kinase Nek4 |
| NEK9 | Serine/threonine-protein kinase Nek9 |
| CIT | Citron Rho-interacting kinase |
| LATS1 | Serine/threonine-protein kinase LATS1 |
| MINK1 | Misshapen-like kinase 1 |
| SIK3 | Serine/threonine-protein kinase SIK3 |
| RPS6KA4 | Ribosomal protein S6 kinase alpha-4 |
| NEK3 | Serine/threonine-protein kinase Nek3 |
| SIK2 | Serine/threonine-protein kinase SIK2 |
| MAST3 | Microtubule-associated serine/threonine-protein kinase 3 |
| STK32C | Serine/threonine-protein kinase 32C |
| ALK | ALK tyrosine kinase receptor |
| EPHB4 | Ephrin type-B receptor 4 |
| PARP2 | Poly [ADP-ribose] polymerase 2 |
| PTK6 | Protein-tyrosine kinase 6 |
| PARP3 | Protein mono-ADP-ribosyltransferase PARP3 |
| CDK2 | Cyclin-dependent kinase 2 |
| CDK8 | Cyclin-dependent kinase 8 |
| BRDT | Bromodomain testis-specific protein |
| MAPKAPK5 | MAP kinase-activated protein kinase 5 |
| MAP3K1 | Mitogen-activated protein kinase kinase kinase 1 |
| CDK18 | Cyclin-dependent kinase 18 |
| CDK10 | Cyclin-dependent kinase 10 |
| TTK | Dual specificity protein kinase TTK |
| PIM2 | Serine/threonine-protein kinase pim-2 |
| PKMYT1 | Membrane-associated tyrosine- and threonine-specific cdc2-inhibitory kinase |
| MKNK2 | MAP kinase-interacting serine/threonine-protein kinase 2 |
| KAT2B | Histone acetyltransferase KAT2B |
| NEK2 | Serine/threonine-protein kinase Nek2 |
| HASPIN | Serine/threonine-protein kinase haspin |
| PIR | Pirin |
| CYP1B1 | Cytochrome P450 1B1 |
| ERN1 | Serine/threonine-protein kinase/endoribonuclease IRE1 |
| MELK | Maternal embryonic leucine zipper kinase |
| COQ8A | Atypical kinase COQ8A, mitochondrial |
| RIOK2 | Serine/threonine-protein kinase RIO2 |
| RPS6KA6 | Ribosomal protein S6 kinase alpha-6 |
| MAP3K20 | Mitogen-activated protein kinase kinase kinase 20 |
| MAPKAPK3 | MAP kinase-activated protein kinase 3 |
| ULK3 | Serine/threonine-protein kinase ULK3 |
| MAP3K21 | Mitogen-activated protein kinase kinase kinase 21 |
| COQ8B | Atypical kinase COQ8B, mitochondrial |
| TNK1 | Non-receptor tyrosine-protein kinase TNK1 |
| BMPR1A | Bone morphogenetic protein receptor type-1A |
| STK17A | Serine/threonine-protein kinase 17A |
| CDK11A | Cyclin-dependent kinase 11A |
| TESK2 | Dual specificity testis-specific protein kinase 2 |
| NLK | Serine/threonine-protein kinase NLK |
| STK35 | Serine/threonine-protein kinase 35 |
| PKN3 | Serine/threonine-protein kinase N3 |
| STK33 | Serine/threonine-protein kinase 33 |
| SBK1 | Serine/threonine-protein kinase SBK1 |
| SLC9A2 | Sodium/hydrogen exchanger 2 |
| CSNK2A3 | Casein kinase II subunit alpha 3 |
| TACC3 | Transforming acidic coiled-coil-containing protein 3 |
| GSPT1 | Eukaryotic peptide chain release factor GTP-binding subunit ERF3A |
| SLC9A7 | Sodium/hydrogen exchanger 7 |
| RPS6KC1 | Ribosomal protein S6 kinase delta-1 |
| TBCK | TBC domain-containing protein kinase-like protein |
| CRABP1 | Cellular retinoic acid-binding protein 1 |
| BCKDK | [3-methyl-2-oxobutanoate dehydrogenase [lipoamide]] kinase, mitochondrial |
| TRPM7 | Transient receptor potential cation channel subfamily M member 7 |
| TRIB3 | Tribbles homolog 3 |
| UHMK1 | Serine/threonine-protein kinase Kist |
| PDIK1L | Serine/threonine-protein kinase PDIK1L |
| STK40 | Serine/threonine-protein kinase 40 |
| SLC9A4 | Sodium/hydrogen exchanger 4 |
| EPHB3 | Ephrin type-B receptor 3 |
| NTRK3 | NT-3 growth factor receptor |
| MAP3K12 | Mitogen-activated protein kinase kinase kinase 12 |
| MAPK11 | Mitogen-activated protein kinase 11 |
| DDR2 | Discoidin domain-containing receptor 2 |
| RARB | Retinoic acid receptor beta |
| PDE4C | cAMP-specific 3′,5′-cyclic phosphodiesterase 4C |
| IDO1 | Indoleamine 2,3-dioxygenase 1 |
| SLC9B1 | Sodium/hydrogen exchanger 9B1 |
| PRAG1 | Inactive tyrosine-protein kinase PRAG1 |

In another aspect the POI is an extracellular POI, which can be selected from the following table:

| | |
|---|---|
| HER2 | Human epidermal growth factor receptor 2 |
| EGFR | Epidermal growth factor receptor |
| HGFR | Hepatocyte growth factor receptor |
| PTK-7 | Protein Tyrosine Kinase 7 |
| CD71 (TfR1) | Cluster of Differentiation 71; Transferrin |
| | receptor protein 1 |
| ApoE4 | Apolipoprotein E4 |
| PD-L1 | Programmed death ligand 1 |

In an aspect, the POI ligand is covalently linked to the heat HSP90 by a chemical linker such as an N-acyl-N-alkyl sulfonamide (NASA) linker, an ortho-dibromophenyl benzoate linker, an electrophilic phenylsulfonate ester group, or an N-sulfonyl pyridine linker.

Also included are pharmaceutical compositions comprising the engineered platelets and a pharmaceutically acceptable excipient.

In an aspect, a method of making an engineered platelet comprises covalently linking a ligand for a POI to an HSP90 to form an to form an HSP90 chimera, and incubating a suspension of platelet cells or platelet-derived microparticles with the HSP90 chimera to load the HSP90 chimera in the platelet cells or platelet-derived microparticles and form the engineered platelets.

In an aspect, the HSP90 chimera is prepared by bridging the POI ligand and an HSP90 ligand by a linker that reacts with a nucleophilic amino acid side chain to generate an HSP90-anchoring chimera, incubating the HSP90-anchoring chimera with HSP90 to transfer and tether the POI ligand to the HSP90, and releasing the HSP90 ligand from the HSP90 to provide the HSP90 chimera.

In an aspect, the linker is an N-acyl-N-alkyl sulfonamide linker, ortho-dibromophenyl benzoate linker, an electrophilic phenylsulfonate ester group, or an N-sulfonyl pyridine linker.

In an aspect, the HSP90 ligand is the HSP90 N-terminal ATPase domain inhibitor PU-H71, Geldanamycin, Tanespimycin 17-AAG, Alvespimycin 17-DMAG, BIIB021, MPC-3100, Radicicol, NVP-AUY922, KW-2478, STA-9090, AT13387, and the like.

In an aspect, a method of degrading an intracellular POI via a ubiquitin-protease system comprises contacting a disease site with the engineered platelet described herein, wherein the POI ligand binds the intracellular POI, and wherein the engineered platelet transfers the HSP90 anchoring chimera to cells at the disease site, thereby binding to and degrading the intracellular POI.

In a further aspect, a method of degrading an extracellular POI via a lysosome-associated pathway comprises contacting a disease site with the engineered platelet of claim 1, wherein the POI ligand binds the extracellular POI, and wherein the engineered platelet releases the HSP-90 anchoring chimera to the extracellular space at the disease site, thereby binding to the extracellular POI and guiding it to the lysosome for degradation.

In an aspect, the disease site is in a patient suffering from a postoperative tumor or a wound-associated disease.

Exemplary post-operative tumors include breast cancer tumors (e.g., triple negative breast cancer tumors), lung tumors, prostate tumors, colorectal tumors, liver tumors, melanoma, ovarian tumors, cervical tumors, pancreatic cancer, and the like.

Exemplary wound-associated diseases include wound infections and chronic wounds.

In an aspect, the engineered platelets are administered by intravenous injection or locoregional administration.

The invention is further illustrated by the following non-limiting examples.

### EXAMPLES

### Materials and Methods

Reagents and antibodies: The information on reagents for chemical synthesis, including names, CAS numbers, and manufacturers, is listed in Table 1. The information on antibodies and other associated biological reagents, including names, manufacturers, and usage, is listed in Table 2. The information on biological kits is listed in Table 3. Information on other chemical or biological reagents used in this study is indicated around the relevant procedures.

TABLE 1
Reagents

| Name | CAS number | Manufacturer |
|---|---|---|
| 1,3-benzodioxole | 274-09-9 | Tokyo Chemical Industry (TCI) |
| N-iodosuccinimide | 516-12-1 | Chem-Impex (Wood Dale, IL, USA) |
| Trifluoroacetic acid (TFA) | 76-05-1 | Tokyo Chemical Industry (TCI) |
| 4,5,6-triaminopyrimidine sulfate | 207742-76-5 | Beantown Chemical Corporation |
| | | (Hudson, NH, USA) |
| Carbon disulfide | 75-15-0 | Thermo Fisher Scientific Inc. |
| neocuproine hydrate | 654054-57-6 | Sigma-Aldrich (St. Louis, MO, USA) |
| Cuprous iodide | 7681-65-4 | Chem-Impex (Wood Dale, IL, USA) |
| Sodium tert-butoxide | 865-48-5 | Chem-Impex (Wood Dale, IL, USA) |
| tert-Butyl 3-bromopropylcarbamate | 83948-53-2 | AmBeed (Arlington Hts, IL, USA) |
| Cesium carbonate | 534-17-8 | AmBeed (Arlington Hts, IL, USA) |
| 4-sulfamoylbenzoic acid | 138-41-0 | Chem-Impex (Wood Dale, IL, USA) |
| 1-ethyl-3-(3- | 7084-11-9 | Chem-Impex (Wood Dale, IL, USA) |
| dimethylaminopropyl)carbodiimide | | |
| hydrochloride (EDC) | | |
| 1-Hydroxybenzotriazole | 2592-95-2 | Ochem Incorporation |
| N,N-diisopropylethylamine | 7087-65-8 | Sigma-Aldrich (St. Louis, MO, USA) |
| 4-Azidobutyric acid | 54447-68-6 | Biosynth Carbosynth (Louisville, KY, USA) |
| 4-(dimethylamino)pyridine (DMAP) | 1122-58-3 | Novabiochem ® |
| Iodoacetonitrile | 624-75-9 | Thermo Fisher Scientific Inc. |
| D-(+)-Biotin | 58-85-5 | Chem-Impex (Wood Dale, IL, USA) |
| (+)-JQ1 PA | 2115701-93-2 | MedChemExpress (Monmouth Junction, NJ, USA) |
| TBTA | 510758-28-8 | AmBeed (Arlington Hts, IL, USA) |
| Copper(II) sulfate pentahydrate | 7758-99-8 | Acros Organics |
| Sodium ascorbate | 134-03-2 | Acros Organics |
| 3-(Bromomethyl)benzene-1- | 79686-36-5 | AmBeed (Arlington Hts, IL, USA) |
| sulfonyl fluoride | | |
| O-(7-Azabenzotriazol-1-yl)- | 148893-10-1 | Chem-Impex (Wood Dale, IL, USA) |
| N,N,Nzzhlxy,Nzzhlxy- | | |
| tetramethyluronium | | |
| hexafluorophosphate (HATU) | | |
| 2-((6R)-4-(4-chlorophenyl)-2,3,9- | 202592-24-3 | AmBeed (Arlington Hts, IL, USA) |
| trimethyl-6H-thieno[3,2- | | |
| f][1,2,4]triazolo[4,3- | | |
| a][1,4]diazepin-6-yl)acetic acid | | |
| BMS-1166 | 1818314-88-3 | AmBeed (Arlington Hts, IL, USA) |
| 2-(2-Propynyloxy)ethylamine | 122116-12-5 | AmBeed (Arlington Hts, IL, USA) |

TABLE 2
Antibodies

| Name | Manufacturer/Catalogue number | Usage (dilution) |
|---|---|---|
| Rabbit anti-HSP90 | Cell Signaling Technology (Danvers, | WB (1:1000) |
| antibody | MA, USA)/4877S | |
| Streptavidin-HRP | Cell Signaling Technology (Danvers, | WB (1:10000) |
| | MA, USA)/3999S | |
| HRP rabbit anti-BRD4 | Abcam (Cambridge, UK)/ab197609 | WB (1:5000) |
| antibody | | |
| HRP mouse anti-beta actin | Abcam (Cambridge, UK)/ab49900 | WB (1:10000) |
| antibody | | |
| Rabbit anti-HSP90α | Cell Signaling Technology (Danvers, | WB (1:1000) |
| antibody | MA, USA)/8165S | |
| Mouse anti-HSP90 | Proteintech Group (Rosemont, IL, | IP (2 μg/sample) |
| antibody | USA)/60318-1-Ig | |
| Normal mouse IgG | Santa Cruz Biotechnology (Dallas, | IP (2 μg/sample) |
| | TX, USA)/sc-2025 | |
| Wheat germ agglutinin | Vector Laboratories (Newark, CA, | IF (7.5 μg/mL) |
| (WGA)-rhodamine | USA)/RL-1022 | |
| Streptavidin-FITC | APExBIO (Houston, TX, | IF (1:25) |
| | USA)/K1081 | |
| Anti-rabbit IgG (H + L), | Cell Signaling Technology | IF (1:1000) |
| F(ab′)2 Fragment (Alexa | (Danvers, MA, USA)/4412S | |
| Fluor ® 488 Conjugate) | | |
| PE anti-mouse CD41 | BioLegend (San Diego, CA, | Flow cytometry |
| antibody | USA)/133905 | (1:80) |
| FITC anti-mouse/rat CD61 | BioLegend (San Diego, CA, | Flow cytometry |
| antibody | USA)/104305 | (1:50) |
| FITC anti-mouse CD9 | BioLegend (San Diego, CA, | Flow cytometry |
| antibody | USA)/124807 | (1:50) |
| PE anti-mouse/rat CD62P | BioLegend (San Diego, CA, | Flow cytometry |
| (P-selectin) antibody | USA)/148305 | (1:40) |
| Rabbit anti-LAMP1 | Cell Signaling Technology | IF (1:250) |
| antibody | (Danvers, MA, USA)/9091T | |
| Rabbit anti-PD-L1 | Invitrogen by Thermo Fisher | WB (1:1000) |
| antibody | Scientific Inc./PA5-20343 | IF (1:100) |
| PE anti-mouse CD3 | BioLegend (San Diego, CA, | Flow cytometry |
| Antibody | USA)/100206 | (1:80) |
| APC anti-mouse CD8a | BioLegend (San Diego, CA, | Flow cytometry |
| Antibody | USA)/100712 | (1:80) |

TABLE 3
Kits

| Name | Manufacturer/Catalogue number | Sample |
|---|---|---|
| QuantTag ™ Biotin Quantitation | Vector Laboratories (Newark, | Mouse |
| Kit | CA, USA)/BDK-2000 | platelets |
| Alanine transaminase | Cayman Chemical (Ann Arbor, | Mouse serum |
| colorimetric activity assay kit | MI, USA)/700260 | |
| Aspartate aminotransferase | Cayman Chemical (Ann Arbor, | Mouse serum |
| colorimetric activity assay kit | MI, USA)/701640 | |
| Urea colorimetric assay kit | Elabscience (Houston, TX, | Mouse serum |
| | USA)/E-BC-K183-M | |
| Mouse heat shock protein 90 | AFG Bioscience (Northbrook, | Mouse |
| ELISA kit | IL USA)/ EK730173 | platelets |
| ELISA MAX ™ Deluxe Set | BioLegend (San Diego, CA, | Mouse tumor |
| Mouse IFN-γ | USA)/430804 | tissue |
| ELISA MAX ™ Deluxe Set | BioLegend (San Diego, CA, | Mouse tumor |
| Mouse TNF-α | USA)/430904 | tissue |

Cells and mice: The murine 4T1 breast cancer cell line, murine LRP-deficient PEA-10 cell line, and human wide-type MDA-MB-231 breast cancer cell line were obtained from ATCC. The human HSP90α-KO MDA-MB-231 breast cancer cell line was kindly provided by Professor Wei Li at Keck School of Medicine of University of Southern California. Luciferase-expressed 4T1 (4T1-Luc) cell line, which was mainly used for in vivo bioluminescence imaging, was purchased from Imanis Life Sciences Inc. Cells were cultured in the CO₂ incubator (Fisher) at 37° C. with 5% CO₂ and 90% relative humidity and were sub-cultured at about 80% confluence. The 8-week-old BALB/c and NSG mice were purchased from the Jackson Laboratory. The animal study protocol was approved by the Institutional Animal Care and Use Committee (IACUC) at the University of Wisconsin-Madison.

General Methods for compound synthesis and analysis: Materials and reagents other than those mentioned in Tables 1 and 2 were purchased from commercial sources and used without further purification. Unless otherwise noted, all reactions were performed with anhydrous solvents at room temperature (˜23° C.) under an inert nitrogen gas atmosphere. During the reaction aftertreatment or product purification phase, the terms “concentrated” and “evaporated” refer to removing the solvent at reduced pressure on a rotary evaporator (Rotavapor® R-100, BUCHI) with a water bath (<50° C.). All the reactions were monitored by thin layer chromatography (TLC) with a suitable developing solvent which were carried out on Merck silica gel plates (60 F254) and visualized with UV lamp 254 nm (MilliporeSigma). The column chromatography employed in this paper was performed on 230-400 mesh silica gel (CAS 7631-86-9, Fisher chemical) with a suitable mobile phase. All nuclear magnetic resonance (NMR) spectra were collected using Bruker Avance III HD 400 MHz NMR Spectrometer and analyzed by MestReNova (Version: 14.0.0-23239, Mestrelab Research S. L.). Chemical shifts (d in ppm) for ¹H spectra are analyzed relative to the residual solvent signals: 7.26 ppm for chloroform-d and 2.50 ppm for dimethyl sulfoxide (DMSO)-d₆. The multiplicities are indicated as follows: s, singlet; d, doublet; t, triplet; q, quartet; m, multiplet; and br s, broad singlet. Chemical shifts (d in ppm) for ¹³C spectra are reported relative to the residual solvent signals: 39.5 ppm for DMSO-d₆. High-resolution mass spectrometry (HRMS) data were obtained by Bruker MaXis II™ Ultra-High Resolution Quadrupole Time-of-Flight MS.

Synthesis and analysis of target molecule 1 (TM-1, HAC-B): The HSP90 ligand part of all the chimeric structures was synthesized based on the previously reported methods with some modifications.

1,2-Diiodo-4,5-(methylenedioxy)benzene (intermediate 1, IM-1). 1,3-benzodioxole (3600 mg, 14.74 mmol) and N-iodosuccinimide (19.9 g, 44.22 mmol) were dispersed in acetonitrile (ACN, 120 mL), and trifluoroacetic acid (TFA, 4,379 μL, 29.48 mmol) was added dropwise. Following stirring at room temperature for 24 h, the solvent of the resulting mixture was evaporated to get a reddish-brown oil. Afterward, the obtained oil was dissolved in 90 mL ethyl acetate (EA) and washed with Na₂SO₃ aqueous solution (3×30 mL), and brine (3×30 mL). The organic phase was then dried over anhydrous Na₂SO₄, filtrated, and concentrated to generate the crude product. The pale-yellow solid was dispersed in cold methanol (MeOH, 50 mL) and stirred at −20° C. for 2 h. Finally, the product (1,080 mg, 2.89 mmol, yield: 20%) was collected as an off-white solid through a pre-cooling filtration system and dried in a vacuum drying oven. IM-1: ¹H NMR (400 MHz, DMSO-d₆) δ 7.48 (s, 2H), 6.06 (s, 2H). ¹³C NMR (101 MHz, DMSO-d₆) δ 149.18, 118.75, 102.75, 97.91.

8-Mercaptoadenine (IM-2). 4,5,6-triaminopyrimidine sulfate (1,339 mg, 6 mmol), NaHCO₃ (2.52 g, 30 mmol), and carbon disulfide (CS₂, 5.22 g, 60 mmol) was dispersed in a combined solvent of H₂O (22.5 Ml) and ethanol (EtOH, 11.25 Ml). After refluxing for 72 h, the orange solution was concentrated to remove the excess CS₂, and acetic acid (AcOH, 1 Ml) was added dropwise to the remaining solution. The yellowish-white precipitate was collected through filtration and dried in a vacuum drying oven. This product (1.078 g, 6.45 mmol, yield: 107%) could be used for the next step directly without further purification.

8-(6-Iodo-benzo[1,3]dioxol-5-ylsulfanyl)adenine (IM-3). A suspension of IM-1 (2,012 mg, 5.4 mmol), IM-2 (600 mg, 3.6 mmol), neocuproine hydrate (81.2 mg, 0.36 mmol), cuprous iodide (CuI, 68.4 mg, 0.36 mmol), and sodium tert-butoxide (414 mg, 4.31 mmol) in anhydrous N,N-dimethylformamide (DMF, 30 mL) was stirred at 110° C. under nitrogen protection for 24 h. Following cooling down to room temperature, the solvent of the reaction suspension was evaporated to get a reddish-brown oil. This crude product was dispersed in a mixture solvent (250 mL) of dichloromethane (DCM), EA, and MeOH at the volume ratio of 2:2:1 (v/v/v). Following refluxing for 2 h, the filtrate was collected, concentrated, and purified by column chromatography on silica gel with a mobile phase of DCM/EA/MeOH at 4:4:1 (v/v/v) to generate an orange solid (IM-3, 306 mg, 0.74 mmol, yield: 21%). IM-3: ¹H NMR (400 MHz, DMSO-d₆) δ 13.20 (s, 1H), 8.08 (s, 1H), 7.51 (s, 1H), 7.21 (s, 2H), 7.01 (s, 1H), 6.09 (s, 2H). ¹³C NMR (101 MHz, DMSO-d₆) δ 155.21, 152.67, 152.48, 149.26, 145.15, 127.16, 120.76, 119.25, 112.66, 102.89, 93.13. HRMS (ESI, m/z): [M+H]⁺ calcd for C₁₂H₉IN₅O₂S⁺413.95162 found 413.95117.

tert-Butyl (3-(6-amino-8-((6-iodobenzo[d][1,3]dioxol-5-yl)thio)-9H-purin-9-yl)propyl)carbamate (IM-4). A solution of IM-3 (306 mg, 0.74 mmol) in anhydrous DMF (10 mL) was added cesium carbonate (412 mg, 1.26 mmol) in one portion and stirred at room temperature for 0.5 h under nitrogen protection. tert-Butyl 3-bromopropylcarbamate (266 mg, 1.11 mmol) was dispersed in anhydrous DMF (3 mL), and then the resulting solution was added to the above suspension dropwise. Following stirring for another 24 h at room temperature, EA (40 mL) was used to dilute the reaction mixture, and deionized water (3×30 mL) was employed to wash the organic phase. After washing with brine (3×30 mL), the EA phase was dried over anhydrous Na₂SO₄, filtered, and concentrated to generate the crude product. The orange solid was purified by column chromatography on silica gel with a mobile phase of DCM/MeOH at 40:1 (v/v) to generate a faint yellow solid (IM-4, 182 mg, 0.32 mmol, yield: 43%). IM-4: ¹H NMR (400 MHz, Chloroform-d) δ 8.34 (s, 1H), 7.31 (s, 1H), 6.91 (s, 1H), 5.99 (s, 2H), 5.65 (s, 2H), 4.27 (t, J=6.5 Hz, 2H), 3.04 (q, J=6.2 Hz, 3H), 1.93 (quintet, J=7.4, 6.8 Hz, 2H), 1.46 (s, 9H). ¹³C NMR (101 MHz, Chloroform-d) δ 155.98, 154.49, 153.17, 151.98, 149.30, 149.10, 127.59, 120.09, 119.30, 112.47, 102.34, 101.76, 40.86, 36.96, 28.47. HRMS (ESI, m/z): [M+H]⁺ calcd for C₂₀H₂₄IN₆O₄S⁺571.061899 found 571.06274.

N-(3-(6-amino-8-((6-iodobenzo[d][1,3]dioxol-5-yl)thio)-9H-purin-9-yl)propyl)-4-sulfamoylbenzamide (IM-5). IM-4 (160 mg, 0.28 mmol) was dissolved in a mixture of DCM (2 mL) and TFA, and then the resulting solution was allowed to stir at room temperature for 4 h. Afterward, the solvent was evaporated, and the residual TFA was totally removed by co-evaporation with toluene (3×1 mL). Following adding anhydrous DMF (3 mL) to disperse the resulting oil, 4-sulfamoylbenzoic acid (67.7 mg, 0.34 mmol), 1-ethyl-3-(3-dimethylaminopropyl)carbodiimide hydrochloride (EDC, 64.5 mg, 0.34 mmol), 1-hydroxybenzotriazole (HOBt, 51.5 mg, 0.34 mmol), N,N-diisopropylethylamine (DIPEA, 146.6 μL, 0.84 mmol) was added to the solution, and the obtained mixture was stirred at room temperature for 12 h. Afterward, EA (30 mL) was used to dilute the reaction solution, and the organic phase was washed with deionized water (3×30 mL) and brine (3×30 mL). The resulting EA phase was dried over anhydrous Na₂SO₄, filtered, and concentrated to generate the crude product. The faint yellow oil was purified by column chromatography on silica gel with a mobile phase of DCM/MeOH at 20:1 (v/v) containing 1% AcOH to generate a white solid (IM-5, 76 mg, 0.12 mmol, yield: 42%). IM-5: ¹H NMR (400 MHz, DMSO-d₆) δ 8.72 (t, J=5.6 Hz, 1H), 8.16 (s, 1H), 8.00-7.95 (m, 2H), 7.90 (d, J=8.5 Hz, 2H), 7.47 (s, 2H), 7.45 (s, 1H), 7.40 (s, 2H), 6.80 (s, 1H), 6.06 (s, 2H), 4.23 (t, J=7.2 Hz, 2H), 3.31-3.25 (m, 2H), 2.01 (quintet, J=7.1 Hz, 2H). ¹³C NMR (101 MHz, DMSO-d₆) δ 165.63, 155.74, 153.50, 151.45, 149.35, 148.87, 146.66, 144.37, 137.81, 128.98, 128.28, 126.10, 120.05, 119.11, 111.37, 102.89, 90.94, 41.75, 37.24, 29.72. HRMS (ESI, m/z): [M+H]⁺ calcd for C₂₂H₂₁IN₇O₅S₂⁺654.008483 found 654.00935.

N-(3-(6-amino-8-((6-iodobenzo[d][1,3]dioxol-5-yl)thio)-9H-purin-9-yl)propyl)-4-(N-(5-((3aS,4S,6aR)-2-oxohexahydro-1H-thieno[3,4-d]imidazol-4-yl)pentanoyl)sulfamoyl)benzamide (IM-6). IM-5 (32 mg, 0.049 mmol) and D-(+)-Biotin (17.95 mg, 0.073 mmol) was dispersed in anhydrous DMF (2 mL) and stirred at room temperature. EDC (28.16 mg, 0.15 mmol), 4-(dimethylamino)pyridine (DMAP, 5.98 mg, 0.047 mmol), and DIPEA (25.6 μL, 0.15 mmol) was added successively, and the resulting suspension was stirred at room temperature under nitrogen protection for 24 h. Then, the solvent of the reaction solution was removed at reduced pressure, and the obtained oil was purified by column chromatography on silica gel with a mobile phase of DCM/EA/MeOH at 2:2:1 (v/v/v) containing 1% AcOH to generate a white solid (IM-6, 19 mg, 0.022 mmol, 44%). IM-6: ¹H NMR (400 MHz, DMSO-d6) δ 12.17 (s, 1H), 8.78 (t, J=5.6 Hz, 1H), 8.16 (s, 1H), 8.05-7.93 (m, 5H), 7.42 (d, J=2.5 Hz, 2H), 6.78 (s, 1H), 6.37 (d, J=15.2 Hz, 2H), 6.06 (s, 2H), 4.28 (dd, J=7.8, 5.1 Hz, 1H), 4.23 (t, J=7.3 Hz, 2H), 4.12-4.04 (m, 1H), 3.65-3.57 (m, 1H), 3.31-3.24 (m, 2H), 3.06-2.97 (m, 1H), 2.80 (dd, J=12.4, 5.1 Hz, 1H), 2.60-2.52 (m, 4H), 2.21 (t, J=7.3 Hz, 2H), 2.01 (p, J=7.2 Hz, 2H), 1.51-1.30 (m, 4H), 1.28-1.14 (m, 2H). ¹³C NMR (101 MHz, DMSO-d6) δ 165.46, 163.14, 162.78, 155.76, 151.45, 149.35, 148.82, 144.31, 129.02, 128.34, 127.99, 120.05, 119.09, 111.23, 102.88, 90.72, 67.49, 61.44, 59.62, 55.71, 37.24, 36.25, 31.24, 29.64, 28.36, 28.25, 25.60, 24.44. HRMS (ESI, m/z): [M+H]⁺ calcd for C32H35IN9O7S3⁺880.086082 found 880.08451.

N-(3-(6-amino-8-((6-iodobenzo[d][1,3]dioxol-5-yl)thio)-9H-purin-9-yl)propyl)-4-(N-(cyanomethyl)-N-(5-((3aS,4S,6aR)-2-oxohexahydro-1H-thieno[3,4-d]imidazol-4-yl)pentanoyl)sulfamoyl)benzamide (TM-1/HAC-B). IM-6 (19 mg, 0.022 mmol) was dissolved in anhydrous DMF (0.5 mL) and stirred at room temperature. After adding DIPEA (18.8 μL, 0.11 mmol) in one portion, iodoacetonitrile (15.6 μL, 0.22 mmol) was added to the resulting solution dropwise. The obtained reaction system was stirred at room temperature under nitrogen protection for 18 h. Then, the solvent of the reaction solution was removed at reduced pressure, and the obtained oil was purified by column chromatography on silica gel with a mobile phase of DCM/EA/MeOH at 4:4:1 (v/v/v) to generate a yellowish-white oil (TM-1/HAC-B, 8.2 mg, 8.9 mol, 41%). TM-1: ¹H NMR (400 MHz, DMSO-d₆) δ 8.87 (t, J=5.6 Hz, 1H), 8.19-8.11 (m, 3H), 8.08 (d, J=8.6 Hz, 2H), 7.45 (d, J=3.9 Hz, 1H), 7.42 (s, 1H), 6.79 (s, 1H), 6.41 (s, 1H), 6.36 (s, 1H), 6.06 (s, 2H), 5.01 (s, 2H), 4.29 (dd, J=7.8, 5.0 Hz, 1H), 4.23 (t, J=7.2 Hz, 2H), 4.10 (ddd, J=7.5, 4.5, 1.7 Hz, 1H), 3.30-3.26 (m, 2H), 3.04 (ddd, J=8.5, 6.3, 4.4 Hz, 1H), 2.83-2.77 (m, 1H), 2.71-2.62 (m, 2H), 2.62-2.53 (m, 3H), 2.02 (p, J=7.2 Hz, 2H), 1.57-1.38 (m, 4H), 1.27 (ddd, J=9.6, 6.7, 3.5 Hz, 2H). ¹³C NMR (101 MHz, DMSO-d₆) δ 172.50, 165.06, 163.18, 151.42, 149.35, 148.85, 140.41, 140.19, 128.96, 128.87, 128.36, 120.03, 119.08, 116.88, 111.29, 102.90, 90.80, 61.41, 59.64, 55.71, 37.29, 35.47, 29.58, 28.42, 28.12, 24.32. HRMS (ESI, m/z): [M+H]⁺ calcd for C34H36IN10O₇S₃⁺919.096981 found 919.1015765.

Synthesis and analysis of TM-2 (iHAC): N-(3-(6-amino-8-((6-iodobenzo[d][1,3]dioxol-5-yl)thio)-9H-purin-9-yl)propyl)-4-(N-(4-azidobutanoyl)sulfamoyl)benzamide (IM-7). IM-5 (114 mg, 0.17 mmol) and 4-azidobutyric acid (33.8 mg, 0.26 mmol) were dispersed in anhydrous DMF (5 mL) and stirred at room temperature. EDC (100.3 mg, 0.52 mmol), DMAP (21.3 mg, 0.17 mmol), and DIPEA (91.2 L, 0.52 mmol) were added successively, and the resulting suspension was stirred at room temperature under nitrogen protection for 24 h. Then, the solvent of the reaction solution was removed at reduced pressure, and the obtained oil was purified by column chromatography on silica gel with a mobile phase of DCM/MeOH at 20:1 (v/v) containing 1% AcOH to generate a white solid (IM-7, 62 mg, 0.081 mmol, 48%). IM-7: ¹H NMR (400 MHz, DMSO-d₆) δ 12.01 (s, 1H), 8.75 (t, J=5.6 Hz, 1H), 8.17 (s, 1H), 8.00-7.94 (m, 5H), 7.42 (s, 2H), 6.78 (s, 1H), 6.06 (s, 2H), 4.23 (t, J=7.2 Hz, 2H), 3.29 (t, J=6.4 Hz, 2H), 3.25 (d, J=6.8 Hz, 2H), 2.26 (t, J=7.3 Hz, 2H), 2.01 (p, J=7.2 Hz, 2H), 1.65 (p, J=7.0 Hz, 2H). ¹³C NMR (101 MHz, DMSO-d₆) δ 172.48, 165.57, 155.75, 153.51, 151.45, 149.35, 148.83, 144.34, 143.11, 138.79, 129.01, 128.12, 127.87, 120.05, 119.09, 111.25, 102.88, 90.74, 50.45, 41.76, 34.87, 33.51, 29.67, 23.92. HRMS (ESI, m/z): [M+H]⁺ calcd for C₂₆H₂₆IN₁₀O₆S₂⁺765.051745 found 765.04796.

N-(3-(6-amino-8-((6-iodobenzo[d][1,3]dioxol-5-yl)thio)-9H-purin-9-yl)propyl)-4-(N-(4-azidobutanoyl)-N-(cyanomethyl)sulfamoyl)benzamide (IM-8). DIPEA (39.8 μL, 0.23 mmol) was added to a solution of IM-7 (35 mg, 0.045 mmol) in anhydrous DMF (250 L) and stirred at room temperature. Then, iodoacetonitrile (33.1 μL, 0.46 mmol) was added, and the reaction mixture was allowed to stir under nitrogen protection and in the dark for 18 h. Flowing diluting the resulting brown solution with EA (10 mL), the organic phase was washed with deionized water (3×10 mL) and brine (3×10 mL). The obtained EA phase was dried over anhydrous Na₂SO₄, filtered, and concentrated to generate the crude product. The pale brown oil was purified by column chromatography on silica gel with a mobile phase of DCM/MeOH at 30:1 (v/v) to generate an off-white solid (IM-8, 16.6 mg, 0.021 mmol, 46%). IM-8: ¹H NMR (400 MHz, DMSO-d₆) δ 8.85 (t, J=5.6 Hz, 1H), 8.15 (d, J=8.9 Hz, 4H), 8.10-8.06 (m, 2H), 7.42 (s, 2H), 6.78 (s, 1H), 6.06 (s, 2H), 5.00 (s, 2H), 4.23 (t, J=7.2 Hz, 2H), 3.32-3.26 (m, 4H), 2.77 (t, J=7.1 Hz, 2H), 2.01 (p, J=7.0 Hz, 2H), 1.77-1.69 (m, 2H). ¹³C NMR (101 MHz, DMSO-d₆) δ 172.01, 165.03, 155.75, 153.51, 151.44, 149.35, 148.84, 144.32, 140.26, 140.21, 129.00, 128.86, 128.38, 120.05, 119.08, 116.76, 111.26, 102.89, 90.74, 50.08, 41.73, 37.30, 34.49, 32.93, 29.60, 23.79. HRMS (ESI, m/z): [M+H]⁺ calcd for C₂₈H₂₇IN₁₁O₆S₂⁺804.062644 found 804.06002.

(S)—N-(3-(6-amino-8-((6-iodobenzo[d][1,3]dioxol-5-yl)thio)-9H-purin-9-yl)propyl)-4-(N-(4-(4-((2-(4-(4-chlorophenyl)-2,3,9-trimethyl-6H-thieno[3,2-f][1,2,4]triazolo[4,3-a][1,4]diazepin-6-yl)acetamido)methyl)-1H-1,2,3-triazol-1-yl)butanoyl)-N-(cyanomethyl)sulfamoyl)benzamide (TM-2/iHAC). The procedures for all of the click chemistry-mediated conjugations in this study between the HSP90 ligand part and the POI ligand part were performed based on a previous method with some modifications. IM-8 (9 mg, 0.0112 mmol) or (+)-JQ1 PA (5.4 mg, 0.0123 mmol) were dissolved in THF (111 or 123 L) to prepare a solution with a concentration of 0.1 mol/L, respectively. After mixing well with each other, 4.4 μL of copper(II) sulfate pentahydrate aqueous solution (0.5 mol/L), 4.4 μL of sodium ascorbate aqueous solution (0.5 mol/L), and 17.6 μL of TBTA DMSO solution (0.125 mol/L) were added to the obtained mixture successively. The resulting suspension was stirred at room temperature for 0.5 h. Then, the solvent of the reaction solution was removed at reduced pressure, and the obtained oil was purified by column chromatography on silica gel with a mobile phase of DCM/EA/MeOH at 5:5:1 (v/v/v) to generate a white solid (TM-2/iHAC, 9.8 mg, 0.008 mmol, 70%). TM-2: ¹H NMR (400 MHz, DMSO-d₆) δ 8.87 (t, J=5.6 Hz, 1H), 8.73 (t, J=5.6 Hz, 1H), 8.15 (s, 1H), 8.13-8.05 (m, 4H), 7.93 (s, 1H), 7.46 (d, J=8.7 Hz, 2H), 7.39 (dd, J=14.0, 6.9 Hz, 5H), 6.79 (s, 1H), 6.05 (s, 2H), 4.96 (s, 2H), 4.53 (t, J=7.2 Hz, 1H), 4.38-4.30 (m, 4H), 4.22 (t, J=7.4 Hz, 2H), 3.28 (m, J=7.4 Hz, 4H), 2.74 (m, J=4.0 Hz, 2H), 2.58 (s, 3H), 2.40 (s, 3H), 2.04-1.98 (m, 4H), 1.60 (s, 3H). ¹³C NMR (101 MHz, DMSO-d₆) δ 171.77, 170.10, 165.02, 163.54, 162.78, 155.74, 155.52, 153.50, 151.44, 150.32, 149.34, 148.84, 145.57, 144.34, 140.19, 140.11, 137.19, 135.67, 132.73, 131.11, 130.65, 130.32, 129.99, 128.97, 128.89, 128.36, 128.19, 123.31, 120.05, 119.08, 102.88, 90.83, 54.33, 48.66, 41.72, 37.96, 37.32, 34.75, 32.93, 29.60, 25.24, 14.54, 14.51, 13.14, 11.77. HRMS (ESI, m/z): [M+H]⁺ calcd for C₅₀H₄₇ClIN₁₆O₇S₃⁺1241.170353 found 1241.16971.

Synthesis and analysis of TM-3 (HAI): 3-(((3-(6-amino-8-((6-iodobenzo[d][1,3]dioxol-5-yl)thio)-9H-purin-9-yl)propyl)amino)methyl)benzenesulfonyl fluoride (TM-3/HAI). The procedures for TM-3/HAI followed a previous method with some modifications. IM-4 (34 mg, 0.06 mmol) was dissolved in DCM (2 mL). TFA (2 mL) was added to the clear solution was added and stirred at room temperature for 4 h to remove the tert-butyloxycarbonyl protection group. Then, the solvent of the reaction solution was removed at the reduced pressure. Toluene (2 mL) was added to the residuals and removed at reduced pressure. The treatment with toluene was repeated 3 times. The resulting oily materials and TEA (16.6 μL, 0.1192 mmol) were dispersed in anhydrous DMF (500 μL). Next, a solution of 1-fluorosulfonyl-3-bromomethyl benzene (13.6 mg, 0.05 mmol) in anhydrous DMF (500 L) was added to the reaction solution dropwise. The mixture was stirred at room temperature for 1 h, and the solvent was removed at the reduced pressure. The obtained oil was purified by column chromatography on silica gel with a gradient mobile phase of DCM/EA/MeOH at 10:10:1 (v/v/v) to 6:6:1 (v/v/v) to generate a white solid (TM-3/HAI, 13.4 mg, 0.02 mmol, 35%). TM-3: ¹H NMR (400 MHz, DMSO-d₆) δ 8.33 (d, J=1.9 Hz, 1H), 8.23 (s, 1H), 8.20 (dd, J=8.0, 2.0 Hz, 1H), 8.08-8.03 (m, 1H), 7.87 (t, J=7.9 Hz, 1H), 7.74 (s, 2H), 7.51 (s, 1H), 6.91 (s, 1H), 6.09 (s, 2H), 4.32 (s, 1H), 4.28 (t, J=7.2 Hz, 2H), 3.04 (s, 2H), 2.55 (t, J=5.5 Hz, 2H), 2.18 (p, J=6.7 Hz, 2H). ¹⁹F NMR (376 MHz, DMSO-d₆) δ−73.91. ¹³C NMR (101 MHz, DMSO-d₆) δ 154.60, 152.07, 151.33, 149.35, 149.11, 138.83, 135.06, 132.35, 132.12, 131.35, 130.39, 129.34, 128.37, 119.95, 119.16, 111.99, 102.97, 92.06, 49.46, 46.13, 44.99, 26.60. HRMS (ESI, m/z): [M+H]⁺ calcd for C₂₂H₂₁FIN₆O₄S₂⁺643.008898 found 643.00781.

Synthesis and analysis of TM-4 (iHACepi):_(R)-2-(4-(4-chlorophenyl)-2,3,9-trimethyl-6H-thieno[3,2-f][1,2,4]triazolo[4,3-a][1,4]diazepin-6-yl)-N-(prop-2-yn-1-yl)acetamide (IM-9). (R)-2-(4-(4-chlorophenyl)-2,3,9-trimethyl-6H-thieno[3,2-f][1,2,4]triazolo[4,3-a][1,4]diazepin-6-yl)acetic acid (80 mg, 0.2 mmol) and propargylamine (15.4 μL, 0.24 mmol) were dispersed in anhydrous DMF (2 mL). Then, after adding TEA (111.2 μL, 0.8 mmol), O-(7-Azabenzotriazol-1-yl)-N,N,Nzzhlxy,Nzzhlxy-tetramethyluronium hexafluorophosphate (HATU, 190 mg, 0.5 mmol) was added in one portion. Following stirring for 0.5 h at room temperature, the reaction mixture was diluted with 30 mL of distilled water. The aqueous phase was extracted with EA (3×20 mL). Afterward, the organic phase was collected, washed with brine, and dried over anhydrous sodium sulfate. The filtrate was collected by filtration and concentrated under reduced pressure. Finally, the residue was purified by column chromatography on silica gel with a mobile phase of DCM/MeOH at 20:1(v/v) to generate a white solid (IM-9, 67 mg, 0.15 mmol, 76%). IM-9: ¹H NMR (400 MHz, DMSO-d₆) δ 8.69 (t, J=5.6 Hz, 1H), 7.50 (d, J=8.8 Hz, 2H), 7.47-7.42 (m, 2H), 4.51 (dd, J=8.6, 5.7 Hz, 1H), 4.07-3.82 (m, 2H), 3.29 (d, J=8.7 Hz, 1H), 3.22-3.20 (m, 1H), 3.20-3.15 (m, 1H), 2.60 (s, 3H), 2.42 (s, 3H), 1.62 (s, 3H). ¹³C NMR (101 MHz, DMSO-d₆) δ 169.90, 163.54, 155.47, 150.34, 137.26, 135.71, 132.82, 131.17, 130.71, 130.30, 129.97, 128.91, 81.81, 73.42, 54.29, 37.84, 28.28, 14.55, 13.16, 11.79. HRMS (ESI, m/z): [M+H]⁺ calcd for C₂₂H₂₁ClN₅OS⁺438.114985 found 438.11508.

(R)—N-(3-(6-amino-8-((6-iodobenzo[d][1,3]dioxol-5-yl)thio)-9H-purin-9-yl)propyl)-4-(N-(4-(4-((2-(4-(4-chlorophenyl)-2,3,9-trimethyl-6H-thieno[3,2-f][1,2,4]triazolo[4,3-a][1,4]diazepin-6-yl)acetamido)methyl)-1H-1,2,3-triazol-1-yl)butanoyl)-N-(cyanomethyl)sulfamoyl)benzamide (TM-4/iHACepi). IM-8 (16 mg, 0.02 mmol) or IM-9 (9.6 mg, 0.022 mmol) were dissolved in THF (199 or 219 L) to prepare a solution with a concentration of 0.1 mol/L, respectively. After mixing well with each other, 7.8 μL of copper(II) sulfate pentahydrate aqueous solution (0.5 mol/L), 7.8 μL of sodium ascorbate aqueous solution (0.5 mol/L), and 31.3 μL of TBTA DMSO solution (0.125 mol/L) were added to the obtained mixture successively. The resulting suspension was stirred at room temperature for 0.5 h. Then, the solvent of the reaction solution was removed at reduced pressure, and the obtained oil was purified by column chromatography on silica gel with a mobile phase of DCM/EA/MeOH at 5:5:1 (v/v/v) to generate a white solid (TM-4/iHACepi, 15.8 mg, 0.013 mmol, 63%). TM-4: ¹H NMR (400 MHz, DMSO-d₆) δ 8.91-8.86 (t, 1H), 8.73 (t, J=5.6 Hz, 1H), 8.15 (s, 1H), 8.13-8.06 (m, 4H), 7.94 (s, 1H), 7.46 (d, J=8.7 Hz, 2H), 7.42-7.36 (m, 5H), 6.79 (s, 1H), 6.06 (s, 2H), 4.96 (s, 2H), 4.53 (t, J=7.2 Hz, 1H), 4.37-4.31 (m, 4H), 4.22 (t, J=7.3 Hz, 2H), 3.29 (m, J=7.1 Hz, 4H), 2.76 (m, J=7.1 Hz, 2H), 2.58 (s, 3H), 2.40 (s, 3H), 2.04-1.99 (m, 4H), 1.60 (s, 3H). ¹³C NMR (101 MHz, DMSO-d₆) δ 171.78, 170.10, 165.02, 163.54, 162.78, 155.74, 155.52, 153.50, 151.44, 150.33, 149.34, 148.84, 145.57, 144.33, 140.19, 140.12, 137.19, 135.67, 132.73, 131.11, 130.65, 130.33, 129.99, 128.97, 128.89, 128.37, 123.31, 120.05, 119.08, 116.74, 111.34, 102.89, 90.75, 54.34, 48.65, 41.72, 37.97, 37.28, 34.74, 32.93, 29.62, 25.84, 25.22, 14.51, 13.13, 11.76. HRMS (ESI, m/z): [M+H]⁺ calcd for C₅₀H₄₇ClIN₁₆O₇S₃⁺1241.170353 found 1241.17147.

Synthesis and analysis of TM-5 (eHAC): (2R,4R)-1-(5-chloro-2-((3-cyanobenzyl)oxy)-4-((3-(2,3-dihydrobenzo[b][1,4]dioxin-6-yl)-2-methylbenzyl)oxy)benzyl)-4-hydroxy-N-(2-(prop-2-yn-1-yloxy)ethyl)pyrrolidine-2-carboxamide (IM-10). BMS-1166 (50 mg, 0.08 mmol) and 2-(2-propynyloxy)ethylamine (9.7 μL, 0.09 mmol) were dispersed in anhydrous DMF (3 mL). Then, after adding TEA (43.4 μL, 0.312 mmol), HATU (74 mg, 0.2 mmol) was added in one portion. Following stirring for 0.5 h at room temperature, the reaction mixture was diluted with 30 mL of distilled water. The aqueous phase was extracted with EA (3×20 mL). Afterward, the organic phase was collected, washed with brine, and dried over anhydrous sodium sulfate. The filtrate was collected by filtration and concentrated under reduced pressure. Finally, the residue was purified by column chromatography on silica gel with a mobile phase of DCM/MeOH at 30:1(v/v) to generate a white solid (IM-10, 45 mg, 0.06 mmol, 76%). IM-10: ¹H NMR (400 MHz, DMSO-d₆) δ 7.95 (t, J=1.7 Hz, 1H), 7.83 (ddt, J=9.3, 6.1, 1.4 Hz, 3H), 7.63 (t, J=7.8 Hz, 1H), 7.46-7.41 (m, 2H), 7.25 (t, J=7.5 Hz, 1H), 7.18 (dd, J=7.7, 1.5 Hz, 1H), 7.06 (s, 1H), 6.93 (d, J=8.2 Hz, 1H), 6.78 (d, J=2.0 Hz, 1H), 6.76 (dd, J=8.2, 2.1 Hz, 1H), 5.30 (s, 2H), 5.22 (s, 2H), 4.74 (d, J=4.9 Hz, 1H), 4.29 (s, 4H), 4.12 (dd, J=5.4, 3.0 Hz, 1H), 4.05 (dd, J=2.4, 0.6 Hz, 2H), 3.67 (d, J=13.3 Hz, 1H), 3.50 (d, J=13.2 Hz, 1H), 3.37-3.35 (m, 2H), 3.21-3.09 (m, 2H), 3.03 (dd, J=9.7, 6.0 Hz, 1H), 2.80 (d, J=10.1 Hz, 1H), 2.41 (dd, J=10.0, 4.8 Hz, 1H), 2.38-2.29 (m, 2H), 2.24 (s, 3H), 1.66-1.56 (m, 1H). ¹³C NMR (101 MHz, DMSO-d₆) δ 173.82, 155.98, 153.78, 143.45, 143.00, 142.13, 139.07, 135.52, 134.86, 134.50, 132.81, 132.22, 131.42, 130.35, 130.25, 128.04, 125.95, 122.60, 120.81, 119.14, 118.19, 117.29, 113.45, 111.96, 101.00, 80.56, 77.66, 70.06, 69.37, 69.24, 68.41, 66.70, 64.58, 57.83, 38.27, 16.34. HRMS (ESI, m/z): [M+H]⁺ calcd for C₄₁H₄₁ClN₃O₇⁺722.262755 found 722.26088.

(2R,4R)—N-(2-((1-(4-((4-((3-(6-amino-8-((6-iodobenzo[d][1,3]dioxol-5-yl)thio)-9H-purin-9-yl)propyl)carbamoyl)-N-(cyanomethyl)phenyl)sulfonamido)-4-oxobutyl)-1H-1,2,3-triazol-4-yl)methoxy)ethyl)-1-(5-chloro-2-((3-cyanobenzyl)oxy)-4-((3-(2,3-dihydrobenzo[b][1,4]dioxin-6-yl)-2-methylbenzyl)oxy)benzyl)-4-hydroxypyrrolidine-2-carboxamide (TM-5/eHAC). IM-8 (12.3 mg, 0.015 mmol) or IM-10 (12.2 mg, 0.017 mmol) were dissolved in THF (153 or 169 L) to prepare a solution with a concentration of 0.1 mol/L, respectively. After mixing well with each other, 6.1 μL of copper(II) sulfate pentahydrate aqueous solution (0.5 mol/L), 6.1 μL of sodium ascorbate aqueous solution (0.5 mol/L), and 24.4 μL of TBTA DMSO solution (0.125 mol/L) were added to the obtained mixture successively. The resulting suspension was stirred at room temperature for 10 min. Then, the solvent of the reaction solution was removed at the reduced pressure, and the obtained oil was purified by column chromatography on silica gel with a gradient mobile phase of DCM/EA/MeOH at 10:10:1 (v/v/v) to 5:5:1 (v/v/v) to generate a white solid (TM-5/eHAC, 15.5 mg, 0.01 mmol, 68%). TM-5: ¹H NMR (400 MHz, DMSO-d₆) δ 8.86 (t, J=5.6 Hz, 1H), 8.16 (s, 1H), 8.12-8.04 (m, 4H), 7.99-7.93 (m, 3H), 7.86-7.78 (m, 3H), 7.60 (t, J=7.8 Hz, 1H), 7.45-7.39 (m, 4H), 7.23 (t, J=7.6 Hz, 1H), 7.17 (dd, J=7.7, 1.5 Hz, 1H), 7.05 (s, 1H), 6.92 (dd, J=8.2, 3.1 Hz, 1H), 6.80-6.77 (m, 2H), 6.74 (dd, J=8.2, 2.1 Hz, 1H), 6.05 (s, 2H), 5.29 (s, 2H), 5.21 (d, J=2.9 Hz, 2H), 4.77 (d, J=4.7 Hz, 1H), 4.43 (d, J=1.8 Hz, 2H), 4.28 (s, 4H), 4.22 (t, J=7.3 Hz, 2H), 4.12 (s, 1H), 4.03 (q, J=7.1 Hz, 2H), 3.67 (d, J=13.2 Hz, 1H), 3.49 (d, J=13.3 Hz, 1H), 3.27 (q, J=6.3 Hz, 4H), 3.12 (q, J=5.9 Hz, 2H), 3.02 (dd, J=9.7, 6.0 Hz, 1H), 2.82-2.78 (m, 1H), 2.41 (dd, J=9.9, 4.7 Hz, 1H), 2.38-2.27 (m, 2H), 2.23 (s, 3H), 1.99 (s, 4H), 1.64-1.56 (m, 1H). ¹³C NMR (101 MHz, DMSO-d₆) δ 173.84, 171.77, 162.78, 156.00, 155.74, 153.78, 153.51, 151.44, 149.34, 148.84, 144.39, 144.34, 143.44, 142.98, 142.11, 140.21, 140.10, 139.05, 135.50, 134.85, 134.48, 132.77, 132.19, 131.53, 131.39, 130.31, 128.97, 128.88, 128.36, 128.02, 125.94, 124.13, 122.59, 120.76, 120.04, 119.13, 119.08, 118.18, 117.27, 116.69, 113.41, 111.94, 111.30, 102.89, 100.99, 90.82, 70.05, 69.36, 69.25, 68.68, 66.64, 64.58, 63.82, 61.95, 60.23, 48.64, 38.40, 36.25, 31.24, 25.13, 21.24, 16.33. HRMS (ESI, m/z): [M+Na]⁺ calcd for C₆₉H₆₆IClN₁₄O₁₃S₂Na⁺1547.300067 found 1547.3006.

Preparation and characterization of the engineered platelets: Platelet isolation: Murine platelets were isolated as described previously in the art with some modifications. In detail, 10% sodium citrate in phosphate buffer saline (PBS) was first added to a 15 mL centrifuge tube and maintained its volume at 10% of the collected whole blood. Nonterminal blood collection from the orbital sinus was employed to obtain the whole blood from BALB/c mice. Afterward, the blood sample was evenly divided into several portions and transferred to 1.5 mL centrifuge tubes. The sample-containing tubes were centrifuged with slow acceleration and deceleration at 100 g for 20 min at room temperature. The transparent supernatants were collected and transferred to the corresponding new 1.5 mL tubes. Then, prostaglandin E1 (PGE1) diluted in PBS was added to each tube to reach a final PGE1 concentration of 1 μM, and the tubes were centrifuged with slow acceleration and deceleration at 100 g for another 15 min at room temperature. After transferring the supernatants to the new tubes, the platelets were isolated through centrifugation with slow acceleration and deceleration at 1,500 g for 20 min at room temperature. PBS containing PGE1 (1 μM) was added to platelet pellets, and all the resuspensions were combined into one tube for further applications. The number of collected platelets was counted by the hemocytometer after dilution. For removing the PGE1, the platelet suspensions were subjected to centrifugation with slow acceleration and deceleration at 1500 g for 20 min at room temperature, and the resulting pellets were washed with fresh PBS.

Preparation of the engineered platelets: To prepare PLT-B, iDePLT, and eDePLT, the same procedures were used except for the different HACs. After collecting and counting the fresh platelets, PBS containing PGE1 (1 μM) was adopted to dilute the platelets to a final concentration of 1×10⁸ platelets/mL. The corresponding HAC (HAC-B, iHAC, or eHAC) stock solution (20 mM) was added to each sample at a final concentration of 10 μM for every 10⁸ platelets. The platelet suspensions containing the corresponding HAC were placed in an orbital shaker with a speed of 50 rpm and incubated for 2 h at room temperature. Afterward, the suspensions were centrifugated with slow acceleration and deceleration at 1,500 g for 20 min at room temperature, and the obtained pellets were washed with fresh PBS twice to remove the HAC and PGE1. Following centrifugation with the same conditions for collecting the platelets, the resulting pellets were resuspended with a relatively smaller volume of fresh PBS, and the obtained platelets in the corresponding suspensions were counted again using the hemocytometer before usage.

Characterization of the engineered platelets: PLT-B was adopted as the representative to characterize the features of the engineered platelets. Flow cytometry was employed to detect the surface maker expression on PLT-B in the resting or activated conditions with naïve platelet (nPLT) as the control. After isolating the fresh murine platelets, partial platelets were treated with HAC-B to generate PLT-B according to the above preparation method, and the remaining platelets were treated with blank DMSO following the same procedures to serve as the control nPLT. For the resting condition, 1×10⁷ platelets (PLT-B or nPLT) were collected and incubated with 100 μL of PBS containing PE-anti-mouse CD41 antibody, FITC-anti-mouse/rat CD61 antibody, and PGE1 (1 μM) or FITC-anti-mouse CD9 antibody and PGE1 (1 μM) in the dark for 30 min at room temperature. Then, the samples were centrifuged with slow acceleration and deceleration at 1500 g for 20 min at room temperature. The pellets were resuspended in PBS and subjected to flow cytometry analysis. Regarding the activated condition, 2×10⁷ platelets (PLT-B or nPLT) were incubated in 500 μL of PBS containing thrombin (MilliporeSigma/605157-1KU) with a concentration of 0.5 U/mL for 30 min. After that, the platelets were collected through centrifugation at 1500 g for 20 min at room temperature and resuspended in 100 μL of PBS containing PE-anti-mouse/rat CD62P antibody. After incubation in the dark for 30 min at room temperature, the samples were centrifuged to collect the platelets and resuspended in PBS for the flow cytometry analysis.

The functionality of the engineered platelets was evaluated through the collagen-binding and aggregation studies on PLT-B. Murine collagen type I/III (Bio-RAD) was dissolved in PBS at 2 mg/mL and added into the confocal dish. A blank dish treated with blank PBS without collagen served as the control group. After incubation at 4° C. overnight, the solution in both dishes was replaced with 2 mL of blocking buffer (1% BSA in PBS), followed by incubation at room temperature for 1 h. Prior to collagen-binding test, the dishes were washed with PBS twice, and 1×10⁷ PLT-B labeled with CellTracker™ green CMFDA dye (Thermo Fisher Scientific/C7025) was added to the pre-treated dishes, respectively. After incubation for 1 min, the dishes were washed with PBS and subjected to Leica SP8 Confocal WLL STED microscopy for observation. As for the aggregation test, 1×10⁷ nPLT and PLT-B were labeled with CellTracker™ green CMFDA dye and suspended in complete medium with thrombin (0.5 U/mL), respectively. The samples were directly subjected to Leica SP8 Confocal WLL STED microscopy for observation.

The immunoprecipitation assays based on the streptavidin-biotin system were used to evaluate the engineering efficiency of HAC-B on platelets. The fresh platelets were isolated from the whole blood of BALB/c mice as described above. PBS containing PGE1 (1 M) was adopted to dilute the platelets to a final concentration of 1×10⁸ platelets/mL, and a total amount of 2×10⁸ platelets (2 mL) was assigned for each sample in one tube. The HAC-B stock solution in DMSO was added to each tube at a final concentration of 2.5, 5, or 10 μM, and then the samples were placed in an orbital shaker with a speed of 50 rpm and incubated for 2 h at room temperature (blank DMSO serve as 0 μM). Meanwhile, other tubes containing the same platelet suspensions were added with HAC-B stock solution at a final concentration of 10 μM, which were shaken at the same conditions for 0.5, 1, 2, 3, or 4 h (the platelet suspension that was centrifugated immediately after HAC-B addition serves as the 0 h group). After incubation, the corresponding samples were centrifuged with slow acceleration and deceleration at 1500 g for 20 min at room temperature, and the obtained pellets were washed with fresh PBS two times to remove the HAC-B. Following the last centrifugation, the pellets were directly lysed on ice with IP lysis buffer (50 mM Tris-HCl, pH 7.4; 150 mM NaCl; 2 mM EDTA; 1% NP-40) containing phenylmethylsulfonyl fluoride (PMSF, 1 mM) and the phosphatase inhibitor cocktail (MedChemExpress/HY-K0011) for 30 min. The lysates were collected and centrifuged at 10,000 g for 10 minutes at 4° C. The total proteins in the supernatants were quantified through the BCA assay, and 10 μL of lysate supernatants were saved as the input samples. To totally pull down the biotin-labeled contents, 44 μL of Pierce™ streptavidin magnetic beads was added to each supernatant, and the resulting mixtures were incubated at 4° C. with a gentle rotation overnight. Subsequently, the supernatant was discarded by placing the tubes on a magnetic rack, and the obtained magnetic beads were washed with IP lysis buffer three times. Finally, an appropriate amount of western blotting (WB) loading buffer was added to each magnetic bead-containing tube and input sample. The boiled samples were subjected to immunoblot analysis for HSP90 and β-actin.

The fresh platelets were incubated with HAC-B at 2.5, 5, 10, 20, and 40 μM for 2 h, and the corresponding lysates were collected using the IP lysis buffer containing PMSF and phosphatase inhibitor cocktail. The biotin-labeled contents in each lysate supernatant were quantified by the QuantTag™ biotin quantitation kit, and the relevant engineering efficiency was calculated based on dividing the amount of biotin-labeled contents by the HAC-B amount added.

As for the release of biotin-labeled contents from activated PLT-B, PLT-B was prepared by incubating the fresh platelets in the PBS containing PGE1 (1 μM) and HAC-B (20 μM) for 2 h. The PLT-B pellets were washed with PBS twice to remove the PGE1 and HAC-B, and then 12×10⁸ PLT-B were resuspended in 600 μL of PBS with or without thrombin at a concentration of 0.25 U/mL, respectively. 100 μL of the PLT-B suspensions were collected from the relevant samples after incubation at 37° C. for 0, 0.5, 1, 2, 3, or 4 h and subjected to a sequentially combined centrifugation at 5,000 g for 5 min and at 11,000 g for 1 min. The supernatants were transferred to the new tubes and subjected to further centrifugation at 2,500 g for 15 min at room temperature. 80 μL of the upper supernatants were transferred to the new tubes and directed treated with 10× permeabilization buffer (1% Triton™ X-100 and 1% sodium citrate in PBS) on ice for 10 min. After centrifugation at 10,000 g for 10 minutes at 4° C., the biotin-labeled contents in each lysate were quantified by the QuantTag™ biotin quantitation kit, and the release percentages were indicated as the released biotin amount in the supernatant out of the total biotin amount in the PLT-B.

Immunoblot analysis: Immunoblot analysis was used to evaluate the expression of BRD4, HSP90, HSP90a, and PD-L1 in 4T1, WT MDA-MB-231, HSP90α-KO MDA-MB-231 or PEA-10 cells after various treatments and also served as the detection method for IP or co-IP experiments. The procedures for preparing IP or co-IP samples were indicated in the relevant experimental parts.

Sample preparation for various cancer cells after free HAC treatment: Various cells, including 4T1, WT MDA-MB-231, HSP90α-KO MDA-MB-231, or PEA-10 cells, were seeded in 6-well culture plates with a density of 5×10⁴ cells in each well and cultured for 24 h. Then, if HAI pretreatment was performed, the cells were incubated in the HAI-containing blank medium at different concentrations for 2 h and washed with PBS twice before the following treatments. The medium in the corresponding wells was replaced with 1 mL of blank medium containing the DMSO vehicle, HACs, or the combinations of various free ligands at the predetermined concentrations. After incubation for the indicated time points, cells were washed with PBS and lysed on ice with the relevant lysis buffer containing PMSF (1 mM) and the phosphatase inhibitor cocktail for 30 min. As for the conditioned medium (CM), the culturing complete medium of the corresponding cancer cells in each well was collected after 24-h incubation and subjected to centrifugation at 2000 rpm for 5 min to obtain the supernatant. The filtrate of the supernatants was collected following filtration over a 0.45 μm filter, serving as the CM. Regarding the cell samples for BRD4 detection, the lysis buffer containing HEPES-KOH (pH=7.5, 10 mM), NaCl (500 mM), EDTA (5 mM), NP-40 (1%), and SDS (0.1%) was used with the indicated PMSF and phosphatase inhibitor cocktail. As for PD-L1 detection, RIPA buffer (Thermo Fisher Scientific/J63306.AP) was used with the indicated PMSF and phosphatase inhibitor cocktail.

Sample preparation for 4T1 cells after DePLT treatment: 4T1 cells were seeded in 6-well culture plates with a density 5×10⁴ cells in each well and cultured for 24 h. The engineered platelets, including iDePLT and eDePLT, were generated by incubating the fresh platelets in PBS containing iHAC or eHAC at 20 μM for 2 h. The fresh nPLT was used as the control. Then, if HAI pretreatment was performed, the cells were incubated in the HAI-containing blank medium at different concentrations for 2 h and washed with PBS twice before the following treatments. The 4T1 cells in each well were first covered with 1.5 mL of blank medium, and then 500 μL of PBS containing certain engineered platelets was supplemented to the corresponding wells. If an activation stimulus was needed, the platelets were treated with thrombin 0.5 U/mL for 0.5 h before supplementing to the 1.5 mL of treating medium. After incubation for the indicated times, cells were washed with PBS and lysed on ice with the relevant lysis buffer containing PMSF (1 mM) and the phosphatase inhibitor cocktail for 30 min. As for cell samples treated with PMPs or supernatants from activated platelets, engineered platelets or nPLT were suspended in 500 μL of PBS containing thrombin at 0.5 U/mL and incubated at 37° C. for 0.5 h. The platelet suspensions were subjected to a sequentially combined centrifugation at 5000 g for 5 min and at 11,000 g for 1 min, followed by further centrifugation at 2500 g for 15 min at room temperature. The corresponding supernatants were supplemented to the 1.5 mL blank medium to treat 4T1 cells in the relevant wells. For the treatments with the presence of epoxomicin (CAS: 134381-21-8, MedChemExpress/HY-13821) or leupeptin (CAS: 103476-89-7, MedChemExpress/HY-18234A), the inhibitors with the predetermined concentration were directly added to the final 2 mL of treating medium.

General procedures for immunoblot analysis: After a 30-min lysis, the lysates were centrifuged at 10,000 g for 10 minutes at 4° C., and the corresponding supernatants were collected, followed by quantification of the total proteins in the supernatant through the BCA assay. Afterward, the proteins in the supernatant were mixed with the loading buffer and heated at 95° C. for 15 min. Equal amounts of proteins from different samples (20 g protein per lane) were loaded into the Bis-Tris gel formulated with Bis-Tis buffer, 30% acrylamide/Bis, 10% ammonium persulfate (APS) solution, and TEMED. The separated proteins were transferred to polyvinylidene fluoride (PVDF) membranes before the membranes were blocked with 5% nonfat milk in PBS-T. Next, the PVDF membranes were incubated with primary antibodies overnight at 4° C., if necessary, followed by the incubation with the goat-anti-rabbit IgG H & L (HRP) secondary antibodies at room temperature for 2 h. Finally, the bands were treated with electrochemiluminescence (ECL) western blot substrate, detected by the iBright™ imaging system and quantified with ImageJ software. 8% Bis-Tris gels were used for BRD4 detection, and 10% gels were adopted to analyze PD-L1.

In-gel fluorescence detection: In-gel fluorescence was mainly used to investigate the internalization of NeutrAvidin™_647 in HAC-B-treated cancer cells.

Sample preparation: 4T1 or WT MDA-MB-231 cells were seeded in 6-well culture plates with a density of 5×10⁴ cells in each well and cultured for 24 h. If HAI pretreatment was performed, the cells were incubated in the HAI-containing blank medium at different concentrations for 2 h and washed with PBS twice before the following treatments. Then, the cells were treated with free HAC-B or the combinations of various ligands at the indicated concentrations for the predetermined time points. Next, the drug-containing medium was removed, and PBS was used to wash the cells twice, followed by incubating the cells in the blank medium containing NeutrAvidin™ DyLight™ 650 (NA-647, Invitrogen/84607) at 0.5 μM for 1 h. Finally, the cells were washed with PBS and lysed on ice with the RIPA buffer containing PMSF (1 mM) and the phosphatase inhibitor cocktail for 30 min. A s for the 4T1 cells cotreated with NA-647 and HAC-B, the cells after 24-h incubation were treated with the blank medium containing NA-647 at 0.5 μM and HAC-B at various concentrations for 2 h before cell lysis.

In-gel fluorescence imaging: After 30-min lysis, the lysates were centrifuged at 10,000 g for 10 minutes at 4° C., and the corresponding supernatants were collected, followed by quantification of the total proteins in the supernatant through the BCA assay. Afterward, the proteins in the supernatant were mixed with ¼ volume of 5× sample buffer formulated by Tris-HCl (pH 6.8, 312.5 mM), sucrose (25%), SDS (10%), bromophenol blue (0.025%), and dithiothreitol (DTT, 250 mM). After incubation for 1 h at room temperature, equal amounts of proteins from different samples (20 g protein per lane) were loaded into the 12% Bis-Tris gel. After the proteins were separated, the gels were subjected to fluorescence imaging with the ChemiDoc™ MP imaging system (Bio-Rad).

Identification of HAC-B-mediated HSP90 labeling: Streptavidin-mediated immunoblot analysis: 4T1 cells were seeded in 6-well culture plates with a density of 5×10⁴ cells in each well and cultured for 24 h. Following washing with PBS twice, the cells were treated with free HAC-B at the indicated concentrations for 2 h. Then, the cells were washed with PBS and lysed on ice with the RIPA buffer containing PMSF (1 mM) and the phosphatase inhibitor cocktail for 30 min. Next, the lysates were centrifuged at 10,000 g for 10 minutes at 4° C., and the corresponding supernatants were collected, followed by quantification of the total proteins in the supernatant through the BCA assay. Subsequently, the proteins in the supernatant were mixed with ¼ volume of 5× sample buffer formulated by Tris-HCl (pH 6.8, 312.5 mM), sucrose (25%), SDS (10%), bromophenol blue (0.025%), and DTT (250 mM). After incubation for 1 h at room temperature, equal amounts of proteins from different samples (20 g protein per lane) were loaded into the 10% Bis-Tris gel. The separated proteins were transferred to PVDF membrane before the membranes were blocked with 5% nonfat milk in PBS-T. The PVDF membrane was incubated with streptavidin-HRP at room temperature for 2 h. As for the determination of the total HSP90 in each sample, the other PVDF membrane was incubated with the rabbit anti-HSP90 antibody (Cell Signaling Technology/4877S) overnight at 4° C., followed by the incubation with the goat-anti-rabbit IgG H & L (HRP) secondary antibodies at room temperature for 2 h. Finally, the bands were treated with ECL western blot substrate and detected by the iBright™ imaging system.

Streptavidin-mediated immunoprecipitation and mass spectrum analysis: 2×10⁶ 4T1 cells were seeded in T75 culture flasks and cultured for 24 h. Then, the medium in the corresponding flasks was replaced with 15 mL of blank medium with DMSO or HAC-B (10 μM). Following incubation for another 2 h, the cells were washed with cold PBS twice and digested with 2 mL trypsin. The cell pellets were collected through centrifugation at 1,200 rpm for 3 min and washed with cold PBS. The final pellets were directly lysed on ice with IP lysis buffer (50 mM Tris-HCl, pH 7.4; 150 mM NaCl; 2 mM EDTA; 1% NP-40) containing PMSF (1 mM) and the phosphatase inhibitor cocktail for 30 min. The lysates were collected and centrifuged at 10,000 g for 10 minutes at 4° C. The total proteins in the supernatants were quantified through the BCA assay, and 20 μL of lysate supernatants were saved as the input samples. To totally pull down the biotin-labeled contents, 150 μL of Pierce™ streptavidin magnetic beads was added to each supernatant, and the resulting mixtures were incubated at 4° C. with a gentle rotation overnight. Subsequently, the supernatant was discarded by placing the tubes on a magnetic rack, and the obtained magnetic beads were washed with IP lysis buffer three times. Finally, an appropriate amount of WB loading buffer was added to each magnetic bead-containing tube and input sample. The boiled samples (20 g protein per lane) were loaded into the 12% Bis-Tris gel. The obtained gel containing separated proteins was subjected to Coomassie brilliant blue (CBB) staining and imaging with the iBright™ imaging system. For immunoblot analysis, the separated proteins were transferred to the PVDF membrane before the membranes were blocked with 5% nonfat milk in PBS-T. The PVDF membrane was incubated with the rabbit anti-HSP90 antibody (Cell Signaling Technology/4877S) overnight at 4° C., followed by the incubation with the goat-anti-rabbit IgG H & L (HRP) secondary antibodies at room temperature for 2 h. Finally, the bands were treated with ECL western blot substrate and detected by the iBright™ imaging system.

For the mass spectrum analysis, an on-bead digestion method was employed to prepare the sample after the streptavidin magnetic beads were collected from the lysates. In brief, beads were resuspended in 100 μL of ammonium bicarbonate (ABC, 50 mM) and incubated with 10 mM DTT at 37° C. for 30 min. Then, the iodoacetamide (IAA) stock solution was directly added to each sample for a final solution with 55 mM. The protein solutions were incubated at 37° C. for another 45 min in the dark. Then trypsin (0.1 g/L in 250 mM ABC solution) was added at a ratio of 1:20 (enzyme/target protein) for digestion overnight at 37° C., which was halted by adding 10% formic acid. The peptides for each sample were dried to zero volume in the speed vacuum concentrator and cleaned up on a ZipTip® C18 tip (Millipore) according to the manufacturer's protocol. 2.0 μL and 4.0 μL of each sample was injected on a 60-min increasing ACN gradient. And the top 10 ms/ms QE method with 7 s was used with dynamic exclusion enabled. A 50-min blank was run between each sample to check for carry-over. The resulting raw files were searched using Proteome Discover 2.4, SEQUEST HT using Uniprot Mus musculus reference proteome with a decoy database added to establish control variability and false discovery rates.

co-Immunoprecipitation assay: 2×10⁶ 4T1 cells were seeded in T75 cell culture flasks and cultured for 24 h. Then, the medium in the corresponding flasks was replaced with 15 mL of blank medium with DMSO or iHAC (10 μM). Following incubation for another 2 h, the cells were washed with cold PBS twice and digested with 2 mL of trypsin. As for the iDePMP treatment, 1×10⁹ iDePLTs were generated as the above engineering method and suspended in 3 mL of PBS containing thrombin (0.5 U/mL), followed by incubation at 37° C. for 0.5 h. The platelet suspensions were evenly divided and subjected to a sequentially combined centrifugation at 5000 g for 5 min and at 11,000 g for 1 min, followed by further centrifugation at 2500 g for 15 min at room temperature. The combined supernatants were supplemented to 12 mL blank medium to treat 4T1 cells in the relevant flasks. After incubation for 6 h, the cells were washed with cold PBS twice and digested with 2 mL of trypsin. The cell pellets were collected through centrifugation at 1200 rpm for 3 min and washed with cold PBS. The final pellets were directly lysed on ice with RIPA buffer containing PMSF (1 mM) and the phosphatase inhibitor cocktail for 30 min. The lysates were collected and centrifuged at 10,000 g for 10 minutes at 4° C. Meanwhile, 25 μL of Pierce™ protein A/G magnetic beads (Thermo Fisher Scientific/88802) was added to each supernatant in microcentrifuge tubes, and the resulting mixtures were incubated at 4° C. with a gentle rotation for 0.5 h. Next, the sample-containing tube was placed on a magnetic rack for 1 minute, and the supernatant was transferred into a new microcentrifuge tube placed on ice, serving as the precleared samples. The total proteins in the precleared samples were quantified through the BCA assay, and 20 μL of the precleared solution samples were saved as the input samples. To each precleared lysate was added an appropriate amount of mouse anti-HSP90 antibody (Proteintech/60318-1-Ig) followed by incubation overnight at 4° C. with gentle rotation. The sample treated with the same amount of normal mouse IgG (Santa Cruz Biotechnology/sc-2025) served as an isotype control group. After that, the pre-washed magnetic bead (equal to 50 μL of magnetic beads) was added to each sample. The resulting mixtures were incubated at room temperature for 2 h with a gentle rotation. Subsequently, the supernatant was discarded by placing the tubes on a magnetic rack, and the obtained magnetic beads were washed with RIPA buffer three times. Finally, an appropriate amount of loading buffer was added to each magnetic bead-containing tube and input sample. The boiled samples were subjected to immunoblot examination for HSP90 and BRD4 proteins.

Fluorescence observation: Membrane fusion between PMPs and cancer cells: 5×10⁴ 4T1 cells were seeded in a confocal dish and cultured for 24 h at 37° C. And 1×10⁸ fresh nPLTs were collected as the above engineering method and suspended in 1 mL of PBS containing PGE1 (1 μM) and wheat germ agglutinin (WGA)-rhodamine (Vector Laboratories/RL-1022) with a concentration of 7.5 g/mL, followed by incubation at 37° C. for 10 min. Next, the labeled platelets were isolated from the incubation solution through centrifugation with slow acceleration and deceleration at 1,500 g for 20 min at room temperature, and the obtained pellets were washed with PBS twice to remove the probe. After the last centrifugation, the pellet was resuspended in 500 μL of PBS containing thrombin (0.5 U/mL) and incubated at 37° C. for 0.5 h. The platelet suspensions were subjected to a sequentially combined centrifugation at 5,000 g for 5 min and at 11,000 g for 1 min, followed by further centrifugation at 2,500 g for 15 min at room temperature. The corresponding supernatants were supplemented to 1.5 mL blank medium to treat 4T1 cells in the confocal dish. The treated 4T1 cells were incubated at 37° C. for 12 h and washed with PBS twice. Subsequently, DiO was diluted in the blank medium to a final concentration of 10 μM, and the obtained solution was used to incubate 4T1 cells at 37° C. for 20 min. The cells were washed with PBS and fixed with 4% paraformaldehyde (PFA) in PBS for 20 min, followed by staining the nucleus with Hoechst 33342. Finally, the cells were subjected to Leica SP8 Confocal WLL STED microscopy for observation.

Distribution of biotin-labeled contents in 4T1 cells: 5×10⁴ 4T1 cells were seeded in a confocal dish and cultured for 24 h at 37° C. 1×10⁸ PLT-B was generated and incubated in 500 μL of PBS with thrombin (0.5 U/mL) for 0.5 h. Then, the PLT-B solution was diluted in 1.5 mL of blank medium and was adopted to cover the 4T1 cells in the dish. After incubation at 37° C. for 6 h, the cells were washed with PBS twice and fixed with 4% PFA at room temperature for 20 min. Then, the cell sample was treated with the permeabilization solution (0.5% Triton™ X-100 in PBS) at room temperature for another 20 min, followed by incubation with the blocking buffer (1% BSA in PBS) at room temperature for 1 h. Before staining with Hoechst 33342, the cell sample was treated with streptavidin-FITC (APExBIO/K1081) overnight at 4° C. Finally, the cells were subjected to Leica SP8 Confocal WLL STED microscopy for observation.

HAC-B-mediated internalization of streptavidin in 4T1 cells: 5×10⁴ 4T1 cells were seeded in a confocal dish and cultured for 24 h at 37° C. Then, the cells were treated with free HAC-B at 5 μM for 2 h. Next, the drug-containing medium was removed, and PBS was used to wash the cells twice, followed by incubating the cells in the blank medium containing streptavidin-FITC at 0.5 μM for 1 h. For cotreatment, 4T1 cells were cotreated with 5 μM HAC-B and 0.5 μM FITC-SA for 1 h. Before staining with Hoechst 33342, the cells were washed with PBS twice and fixed with 4% PFA at room temperature for 20 min. Finally, the cells were subjected to Leica SP8 Confocal WLL STED microscopy for observation.

Colocalization between internalized NeutrAvidin™ and lysosome maker: 5×10⁴ WT MDA-MB-231 cells were seeded in a confocal dish and cultured for 24 h at 37° C. Then, the cells were treated with free HAC-B at 5 μM for 2 h. Next, the drug-containing medium was removed, and PBS was used to wash the cells twice, followed by incubating the cells in the blank medium containing NA-647 at 0.5 μM for 1 h. Subsequently, the cells were washed with PBS twice and fixed with 4% PFA at room temperature for 20 min. Subsequently, the cell sample was treated with the permeabilization solution (0.5% Triton™ X-100 in PBS) at room temperature for another 20 min, followed by incubation with the blocking buffer (1% BSA in PBS) at room temperature for 1 h. The cell sample was incubated with the rabbit anti-LAMP1 antibody (Cell Signaling Technology/9091T) overnight at 4° C., followed by the incubation with the Alexa Fluor®488-conjugated anti-rabbit IgG H & L secondary antibodies at room temperature for 2 h. After staining with Hoechst 33342, the cells were subjected to Leica SP8 Confocal WLL STED microscopy for observation.

PD-L1 distribution in WT MDA-MB-231 cells: 5×10⁴ WT MDA-MB-231 cells were seeded in a confocal dish and cultured for 24 h at 37° C. Then, the cells were treated with eHAC or the combination of PU-H71 plus BMS-1166 at 0.5 μM for 2 h. Subsequently, the cells were washed with PBS twice and fixed with 4% PFA at room temperature for 20 min. The following procedures were the same as the above colocalization observation except for replacing the anti-LAMP1 antibody with rabbit anti-PD-L1 antibody (Invitrogen/PA5-20343).

Quantitative proteomics analysis: Cell culture and proteomic sample preparation: The procedures for sample preparation were similar to methods described in the art. In brief, 4T1 cells (2×10⁶ for each sample) were seeded in T75 cell culture flasks and cultured for 24 h. Then, the medium was replaced with 12 mL of blank medium. Meanwhile, 1×10⁹ iDePLTs were generated and incubated in 3 mL of PBS with thrombin (0.5 U/mL) for 0.5 h. As for the control group, the same amount of nPLT was suspended in 3 mL of PBS with thrombin (0.5 U/mL) for 0.5 h. Next, the platelet suspensions were diluted in 12 mL of blank medium, and the platelet-containing medium was adopted to incubate the corresponding cells in the flasks. After incubation at 37° C. for 12 h, cells were washed with cold PBS twice and digested with trypsin. The cell pellets were collected through centrifugation at 1,200 rpm for 3 minutes at 4° C., followed by washing with PBS twice.

For the proteomic sample preparation, cell pellets placed on the ice were completely resuspended with a cold ammonium bicarbonate (100 mM) solution. Then, a 23-gauge needle attached to a 1 mL syringe was used to homogenize each cell pellet sample by drawing the cells into the syringe and then fully depressing the plunger. After being homogenized six times, the lysates were centrifugated at 15,000 g for 20 min at 4° C. The supernatants were transferred to new tubes, and a small aliquot of each lysate supernatant was collected for the BCA assay. Based on the quantification results, an appropriate volume of lysate supernatant containing 20 g proteins was collected and incubated with DTT (10 mM) at 37° C. for 30 min. Then, the IAA stock solution was directly added to each sample at a final concentration of 55 mM. The protein solutions were incubated at 37° C. for another 45 min in the dark. Then trypsin (0.1 g/L in 250 mM ammonium bicarbonate solution) was added at a ratio of 1:20 (enzyme/target protein) for an 18-h digestion, which was halted by adding 10% formic acid. The peptides for each sample were dried to zero volume in the speed vacuum concentrator and cleaned up on a ZipTip® C18 tip (Millipore) according to the manufacturer's protocol.

LC-MS/MS analysis: A Trap/Elute LC method was performed with a trap column (nanoEase™ M/Z Symmetry C18 Trap Column, 100A, 5 μm 180 μm×20 mm, Waters) and an analytical column (nanoEase™ M/Z Peptide BEH C18 300A, 1.7 μm 175 μm×150 mm, Waters). All samples were resuspended in 0.1% formic acid in water at ˜0.1 μg/μL, and 2 μL total peptide mass solution was injected twice. The mobile phases (A: water with 0.1% formic acid and B: acetonitrile with 0.1% formic acid) were driven at 30 μL/min for 0.5 min with a constant ratio of 99:1 (A/B) for the trap course. As for the eluting phase, an 80-min increasing gradient elution was performed at 0.43 PL/min. Mobile phase B was increased from the initial 1% to 15% in 44 min, followed by an increase to 23% at 62 min. Then, the ratio of B was elevated to 95% at 72 min after a ramp to 30% at 67 min. A wash with 95% B lasted 3 min, and the flow was ramped back to 2% B in 1.5 min. Finally, the column was re-equilibrated at 2% B for 3.5 min, for a total 80-min run. Eluted peptides were analyzed by timsTOF Pro with a PASEF scan mode for DDA acquisition spanning 100-1,700 m/z with 10 PASEF ramps. The TIMS settings were 100 ms ramp and accumulation time (100% duty cycle) with a ramp rate of 9.42 Hz, leading to a total cycle time of 1.17 s. A 145,000 target intensity with a 1,750 intensity threshold was adopted for linear precursor repetitions, and active exclusion was enabled with a 0.28 min release. The collision energy was a base of 1.6 1/KO [V·s/cm²] set at 59 eV and 0.6 1/KO [V·s/cm²] at 20 eV. Isolation widths were set at 2 m/z at <700 m/z and 3 m/z at >800 m/z. A 50 min blank run was performed between each sample to check for carry-over.

Data analysis: FragPipe (version 18.0) was employed to search the resulting raw files against the UniProt Mus musculus reference proteome (UP000005640). The strict trypsin digestion was set with 2 missed cleavages, and the precursor and fragment mass error was ±20 ppm. 1% was adopted as the false discovery rate for protein and peptide levels. Quantification was based on the LFQ algorithm in Ionquant using a match between runs and normalization between replicates. The ANOVA model is used for statistical significance analysis to determine the significant differences between different treatments.

Detection of the released HSP90 from activated platelets: The enzyme-linked immunosorbent assay was employed to evaluate the HSP90 level in the PMP and free extracellular HSP90 from activated platelets. In this study, both the platelet-derived microparticles and the potential exosomes were regarded as the PMPs, which were isolated following a similar method described in the art. The fresh platelets were isolated from the whole blood of BALB/c mice as described above. 2×10⁸ platelets were suspended in 500 μL of PBS containing thrombin (0.5 U/mL) for each sample and placed at 37° C. After incubation for the indicated time points, the supernatants were collected through a sequentially combined centrifugation at 5,000 g for 5 min and at 11,000 g for 1 min, which were transferred to the new tubes and subjected to centrifugation at 2,500 g for 15 min at room temperature. The obtained supernatants were collected and centrifugated at 20,000 g for 40 min at room temperature. The pellets were isolated and placed on ice for further use. Meanwhile, the relevant supernatants were subjected to centrifugation at 100,000 g for 1 h at 4° C. and the isolated pellets combined with the corresponding pellets obtained last step were directly lysed on ice with RIPA buffer containing PMSF (1 mM) and the phosphatase inhibitor cocktail for 30 min. The lysates were collected and centrifuged at 10,000 g for 10 minutes at 4° C. The HSP90 levels in the lysates and the supernatants after the last centrifugation were quantified through the Mouse heat shock protein 90 ELISA kit (AFG Bioscience/EK730173).

Evaluation of treatment efficacy and biosafety in post-surgical 4T1 tumor-bearing mice: Post-surgery BALB/c mice with orthotopic 4T1-Luc-breast cancer were used to evaluate the tumor recurrence, metastasis, and biosafety profiles after iDePLT or eDePLT treatments. The treatment efficacy of iDePLT was also studied in the post-surgery NSG mice with orthotopic patient-derived xenografts (PDX) breast cancer.

Establishment of post-surgery models with BALB/c mice: 1×10⁶ 4T1-Luc cells were suspended in a PBS containing 50% Matrigel® (Corning®/354234). Female 8-week-old BALB/c mice were anesthetized and injected with 50 μL of the cell suspensions into the fourth mammary gland fat pads. After the tumor volumes reached about 100-200 mm³, the tumor-bearing mice were anesthetized and placed under the microscope, followed by surgical resection to totally remove the visible tumor tissues. Following wound closure, the post-surgery mice were randomly assigned to different treatment groups.

Evaluation of treatment efficacy and biosafety in post-surgery BALB/c mice: As for the treatments with platelets, the platelets were freshly isolated from the healthy female BALB/c mice and suspended in PBS for intravenous (i.v.) injection after the necessary engineering. The free iHAC and eHAC were intraperitoneally (i.p.) injected after dispersing in the formulation of 10% DMSO, 40% polyethylene glycol 300 (PEG300), 5% Tween TM-80, and 45% saline. From the same day after surgery, the mice were anesthetized and i.p. injected with D-luciferin potassium salt (150 mg/kg) in 100 μL in PBS. The bioluminescence of post-surgery mice in each group was detected by Perkin Elmer IVIS every five days from the day of surgery, and intensity was analyzed by Living Image Software v.4.3.1. From the first day after surgery, the mice were i.v. injected with PBS, nPLT (2×10⁸ platelets), iDePLT (2×10⁸ platelets), iDePLTepi (2×10⁸ platelets), and free iHAC (5 mg/kg), or with PBS, nPLT (2×10⁸ platelets), eDePLT (2×10⁸ platelets), and free eHAC (5 mg/kg) every three days for four times, respectively. During the treatment course, the body weight of each post-surgery mouse was monitored daily. As for survival analysis, the death or recurrent tumor volume over 2,000 mm³ was set as the endpoint for these tumor-bearing mice.

Flow cytometry analysis and ELISA assay were adopted to study the infiltrated effector T cells and immune-associated cytokines within the tumor tissues. The same post-surgery mouse models were established and randomly divided into 4 groups (n=5), which were i.v. injected with PBS, nPLT (2×10⁸ platelets), eDePLT (2×10⁸ platelets), and free eHAC (5 mg/kg) every three days for four times starting from day 1 post-surgery. After 2 weeks of the last treatment, the tumor tissues were isolated, weighed, and cut into small pieces, which were then soaked in 2 mL blank medium containing collagenase (MilliporeSigma/C5138) and incubated at 37° C. for 1 h. The cell suspensions were centrifugated at 1200 rpm for 3 min, and 200 μL of the corresponding supernatants were collected and subjected to ELISA analysis for IFN-γ and TNF-α. The single cell suspensions were collected through filtration of the suspension over 40 m nylon cell strainer (Falcon®/352340). For the staining, 1×10⁶ cells for each sample were collected and incubated with 100 μL of PBS containing PE anti-mouse CD3 antibody and APC anti-mouse CD8a antibody in the dark for 30 min at 4° C. Then, the samples were centrifuged at 2,000 rpm for 5 min at room temperature. The pellets were resuspended in PBS and subjected to the flow cytometry analysis.

To further verify the biosafety profile of iDePLT or eDePLT, the same post-surgery mouse models were established and randomly divided into 2 groups (n=6). The mice in two treatment groups were i.v. injected with PBS and iDePLT (2×10⁸ platelets) or with PBS and eDePLT (2×10⁸ platelets), respectively. Following the same dosing schedules as above, the major organs were harvested for H & E staining at 72 h after the last administration (n=3). The hematological and blood chemical analysis for iDePLT-treated post-surgery mice was performed after 1 week of the last treatment. The whole blood samples were collected and subjected to the Abaxis HM5 Complete Blood Count Analyzer. The serums were isolated and analyzed using the alanine transaminase colorimetric activity assay kit, aspartate aminotransferase colorimetric activity assay kit, and urea colorimetric assay kit. For the hematological measurement for eDePLT-treated mice, the whole blood samples were collected after 2 weeks of the last treatment and analyzed using the Abaxis HM5 Complete Blood Count Analyzer.

Evaluation of treatment efficacy in post-surgery NSG mice: First, the orthotopic breast cancer was established in female 8-week-old NSG mice with the TM00096 tumor samples. In brief, the patient tumor sample was cut into small pieces and suspended in a PBS containing 50% Matrigel®. NSG mice were first anesthetized and subcutaneously injected with the above suspension at multiple sites on the back. After the tumor volume reached 500˜800 mm³, the tumors were then passaged to another 14 female 8-week-old NSG mice. As the tumor volumes reached about 100 mm³, the tumor-bearing NSG mice were anesthetized and placed under the microscope, followed by surgical resection to totally remove the visible tumor tissues. Following wound closure, the post-surgery mice were randomly assigned to with PBS (n=4), iDePLT (n=5), and iDePLTepi (n=5) groups. From the first day after surgery, the mice were i.v. injected PBS, iDePLT (2×10⁸ platelets), and iDePLTepi (2×10⁸ platelets) every three days for four times. During the treatment course, the body weight of each post-surgery mouse was monitored every other day. After 2 weeks of the last treatment, the tumor tissues were isolated, weighed, and photographed.

Statistical analysis: Statistical analysis was performed using GraphPad Prism (version 8). All in vitro data are presented as mean±standard deviation (SD). All in vivo data are presented as mean±standard error of mean (SEM). Unpaired student's t-test was used for between two-group comparison and ANOVA was used to perform multiple-group analysis. Survival studies were analyzed using Log-rank test. Differences were considered statistically significant if *P<0.05, **P<0.01, ***P<0.001.

### Example 1: IHAC Degrades the Intracellular POI by Covalently and Non-Covalently HSP90 Binding

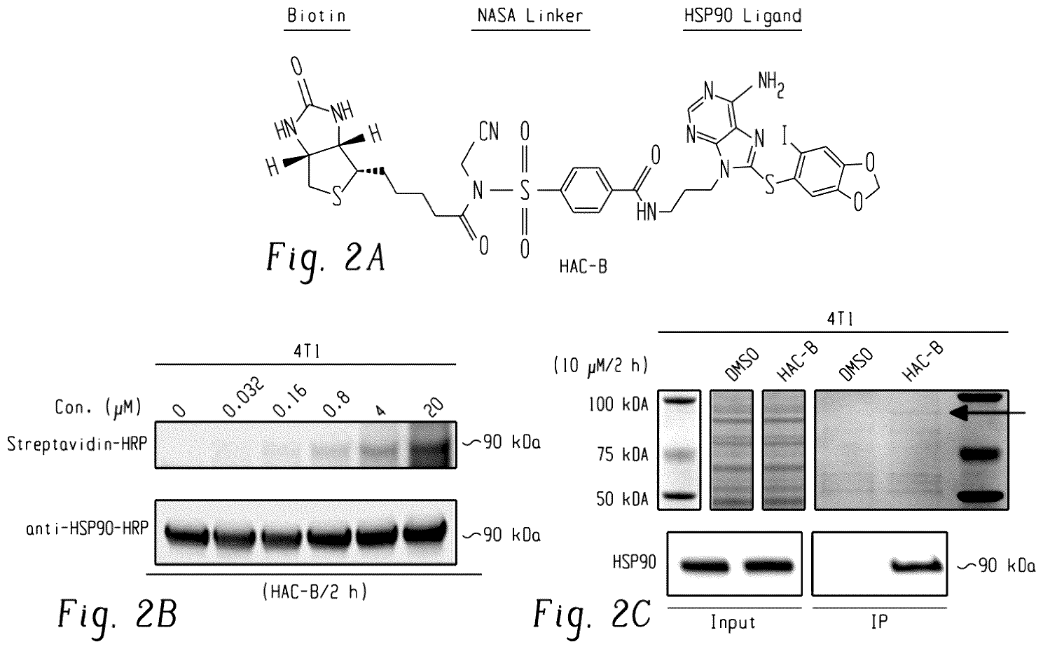

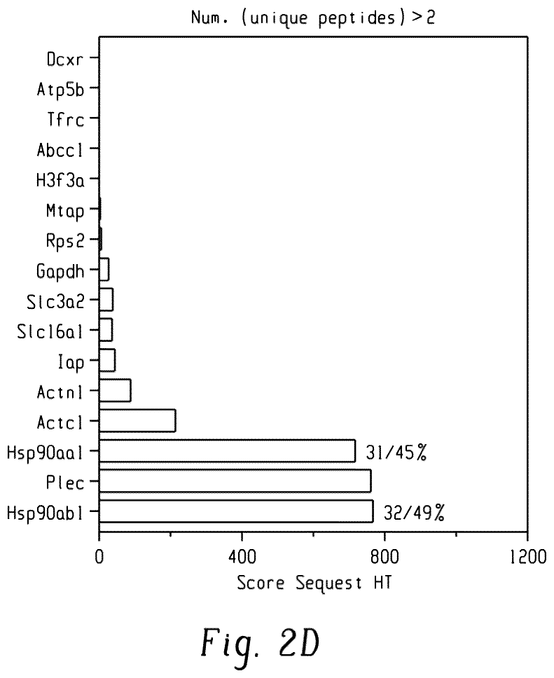

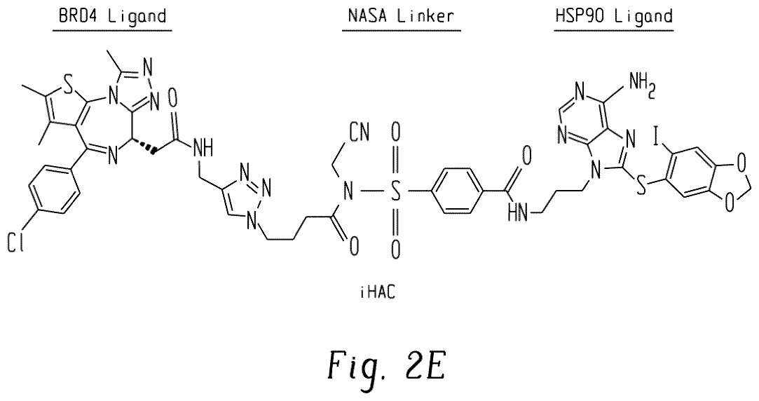

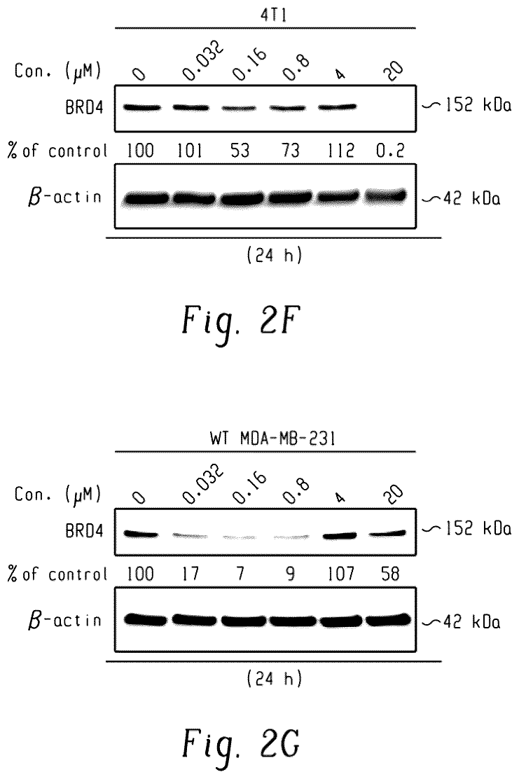

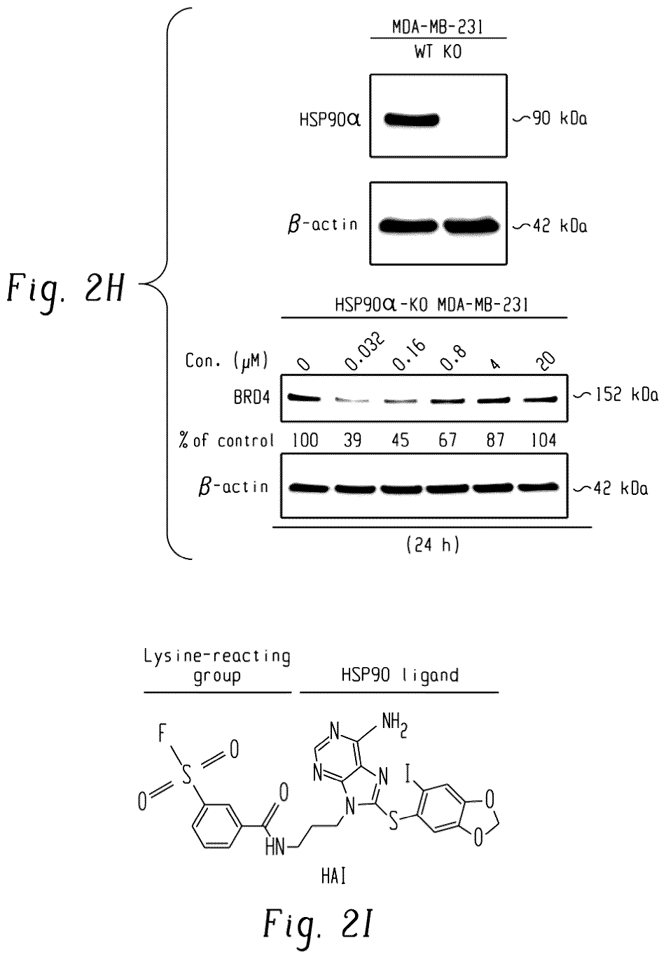

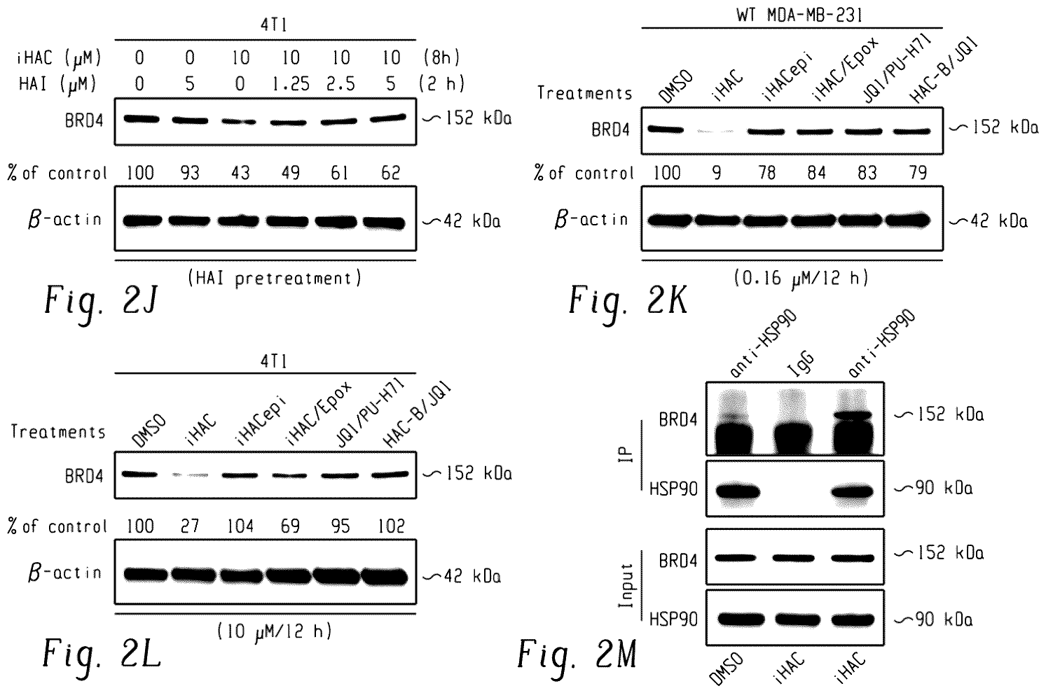

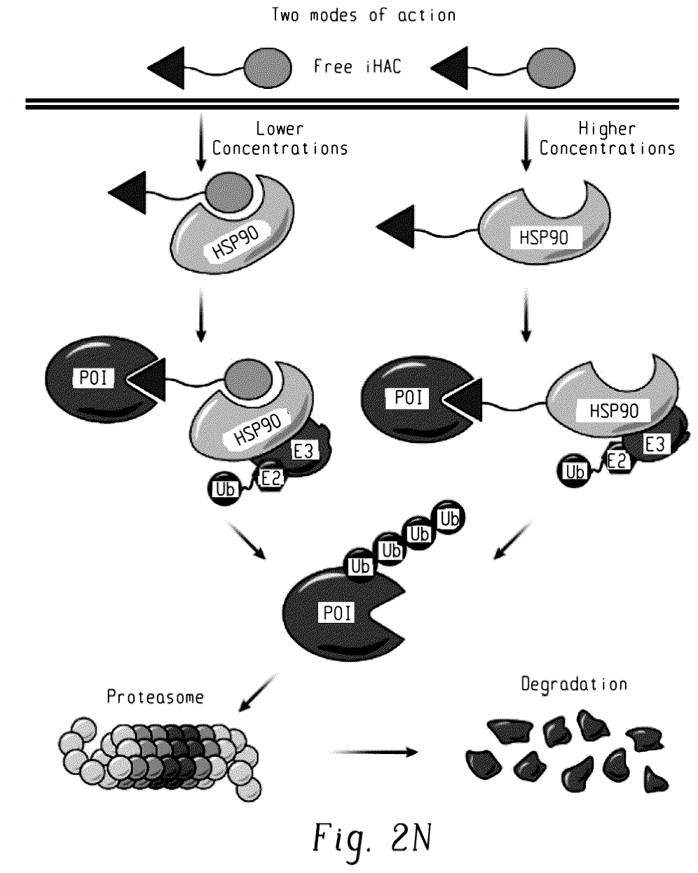

The prerequisite to implementing the strategy described herein is to rapidly and selectively label HSP90 without changes in the physicochemical and biological features of live platelets. A ligand-directed covalent labeling approach, mediated by the N-acyl-N-alkyl sulfonamide (NASA) linker, was employed to covalently modify HSP90 with POI ligand but without retaining the HSP90 warhead in the binding pocket under a mild condition. To verify this method can label HSP90 in a specific and practicable way, a reversible inhibitor that binds to the HSP90 N-terminal ATPase domain, PU-H71, was used as the warhead and first conjugated with (+)-biotin to generate the biotin-tagged HSP90-anchoring chimera (HAC-B, FIG. 2A and Scheme 1). Next, 4T1 cells, a murine breast cancer cell line, were treated with HAC-B and the resulting cell lysates were collected for immunoblot analysis. The results demonstrated that the HAC-B modified HSP90 within 2 h in a concentration-dependent manner (FIG. 2B). The streptavidin-mediated immunoprecipitation (IP) assay was then performed for the HAC-B-treated cell lysate. Compared to the sample without the treatment, an additional HSP90 band appeared in the gel, consistent with the immunoblot analysis results (FIG. 2C). Finally, the protein identification on IP samples through the mass spectrum indicated that the designed chimera could bind to both isoforms of HSP90, HSP90α (Hsp90aa1) and HSP90β (Hsp90ab1), with a favorable selectivity (FIG. 2D).

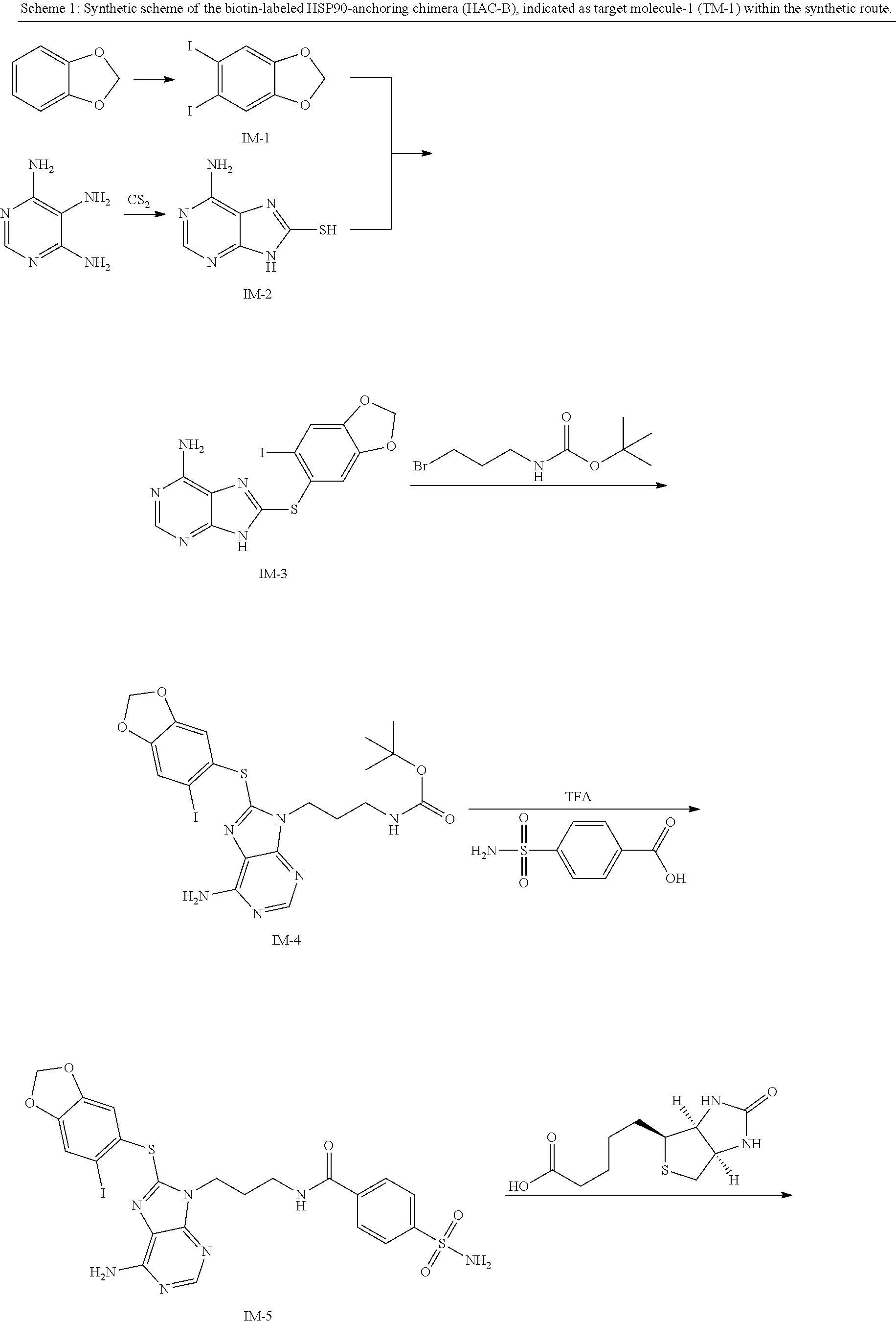

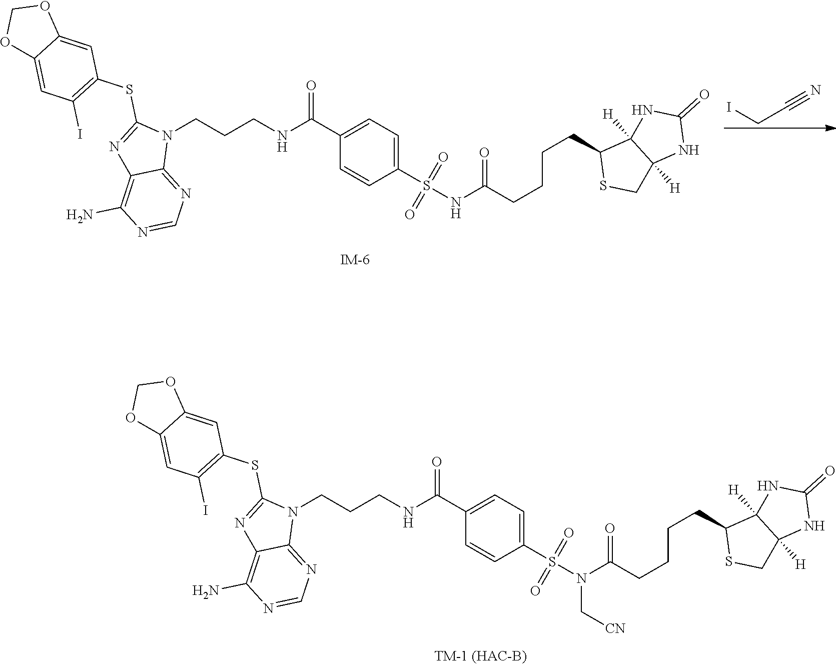

Afterward, the biotin of HAC-B was replaced with the BRD4 ligand, (+)-JQ-1, to construct the BRD4-targeting HSP90-anchoring chimera (designated iHAC) with an expectation that iHAC could tether the BRD4 ligand to HSP90 (FIG. 2E and Scheme 2). BRD4, a member of bromodomain and extra terminal proteins, has been identified as a key treatment target for triple-negative breast cancer, which has poor treatment outcomes in the clinic, even with aggressive chemotherapy and immunotherapy. 4T1 cells were incubated with iHAC at different concentrations for 24 h. This chimera was found to efficiently reduce BRD4 expression at 0.16 μM and 20 μM (FIG. 2F). A similar downregulation trend was observed in iHAC-treated wide type (WT) MDA-MB-231 cells (FIG. 2G), a human TNBC cell line. Furthermore, iHAC at the two effective concentrations induced BRD4 downregulation inside 4T1 and WT MDA-MB-231 cells in a time-dependent manner, respectively (data not shown). According to the reported TPD technologies and involvement of HSP90 in protein processing, it was reasonable to speculate that free iHAC could bring HSP90 and BRD4 into proximity and redirect the latter to undergo degradation. Besides, in both cell lines, a typical “hook effect”, in which the saturating doses trend to binary binding between chimera with either effector or POI, was observed using the relatively lower concentrations and was reversed by the treatment at 20 μM. Since HAC-B over 0.8 μM could label HSP90 with higher efficiency (FIG. 2B), it was inferred that HSP90 could be covalently labeled with iHAC at higher concentrations and then realize TPD through binary complex between pre-labeled HSP90 and BRD4. As for the lower effective concentrations, iHAC was able to non-covalently bind to HSP90 and degrade BRD4 through the ternary complex mechanism. Next, given the binding of the designed chimera to both isoforms, it was attempted to establish the HSP90α- and HSP90β-knock-out (KO) cell line to confirm the dependency of iHAC-mediated BRD4 degradation on HSP90. Since the MDA-MB-231 cells operated for HSP900-KO suffer from low viability, the obtained HSP90α-KO MDA-MB-231 cells were used to elucidate the degradation mechanism mediated by iHAC. After treating HSP90α-KO MDA-MB-231 cells with iHAC for 24 h, the results indicated that HSP90a deficiency substantially compromised the BRD4 degradation efficacy of iHAC within both lower and higher concentration ranges (FIG. 2H). Due to the roles of HSP900 as a molecular chaperone in protein regulation, iHAC partially maintains the degradation potency in HSP90α-KO MDA-MB-231 cells, and the concentration-dependent efficacy also exhibited the typical “hook effect”. Further, a covalent inhibitor derived from PU-H71 was synthesized (FIG. 2I and Scheme 3), which would occupy the same binding pocket and react with the same lysine residue of HSP90 as iHAC. This HSP90-anchoring inhibitor (designated HAI) was used to pretreat 4T1 cells at various concentrations, and then inferior BRD4 degradation efficacy was observed after the iHAC treatment (FIG. 2J). Thus, the BRD4 degradation was largely affected by genetic knock-out and chemical blockage against HSP90, suggesting the involvement of HSP90 in iHAC-mediated downregulation. Additionally, (R)-(−)-JQ-1 with an inverted stereocenter from (+)-JQ-1 was used to generate an inactive control structure for iHAC (Scheme 4), designated iHACepi, which shares the identical physicochemical feature with its counterpart but loses the binding ability to BRD4. Whether at lower or higher effective concentrations, compared to iHAC, there was almost no substantial BRD4 degradation in WT MDA-MB-231 and 4T1 cells after the treatments with iHACepi, and the combinations of (+)-JQ-1 plus PU-H71 or HAC-B plus (+)-JQ-1, respectively (FIGS. 2K and 2L). The failure of these treatments emphasized the necessity of iHAC simultaneously capturing HSP90 and BRD4 during the degradation procedures. Coupled with the rescue effect induced by proteasome inhibitor epoxomicin (Epox), iHAC at lower or higher effective concentrations triggered BRD4 degradation through UPS machinery. Finally, the co-immunoprecipitation (co-IP) assay was employed to verify the complex formation between HSP90 and BRD4 induced by iHAC at 10 μM, and immunoblot analysis was used to detect the BRD4 presence in the proteins co-precipitated with anti-HSP90 antibody. Although BRD4 was reported to be a guest protein of HSP90, the iHAC could considerably enhance the interaction between BRD4 and HSP90 (FIG. 2M). Combined with all MOA validation results, it revealed that a binary complex was formed between BRD4 and HSP90 after iHAC treatment at a relatively higher concentration. Besides, with iHAC treatments at various concentrations, all the cancer cells maintained a relatively stable HSP90 expression (data not shown), indicating that covalent labeling on HSP90 and BRD4 degradation induced by iHAC would not cause HSP90 consumption. While free iHAC with lower concentrations could non-covalently bring HSP90 and BRD4 into proximity and trigger BRD4 degradation, the higher-concentration iHAC was demonstrated to covalently tether the BRD4 ligand to HSP90, so the pre-labeled HSP90 could capture BRD4 and initiate the following UPS-associated degradation procedures (FIG. 2N).

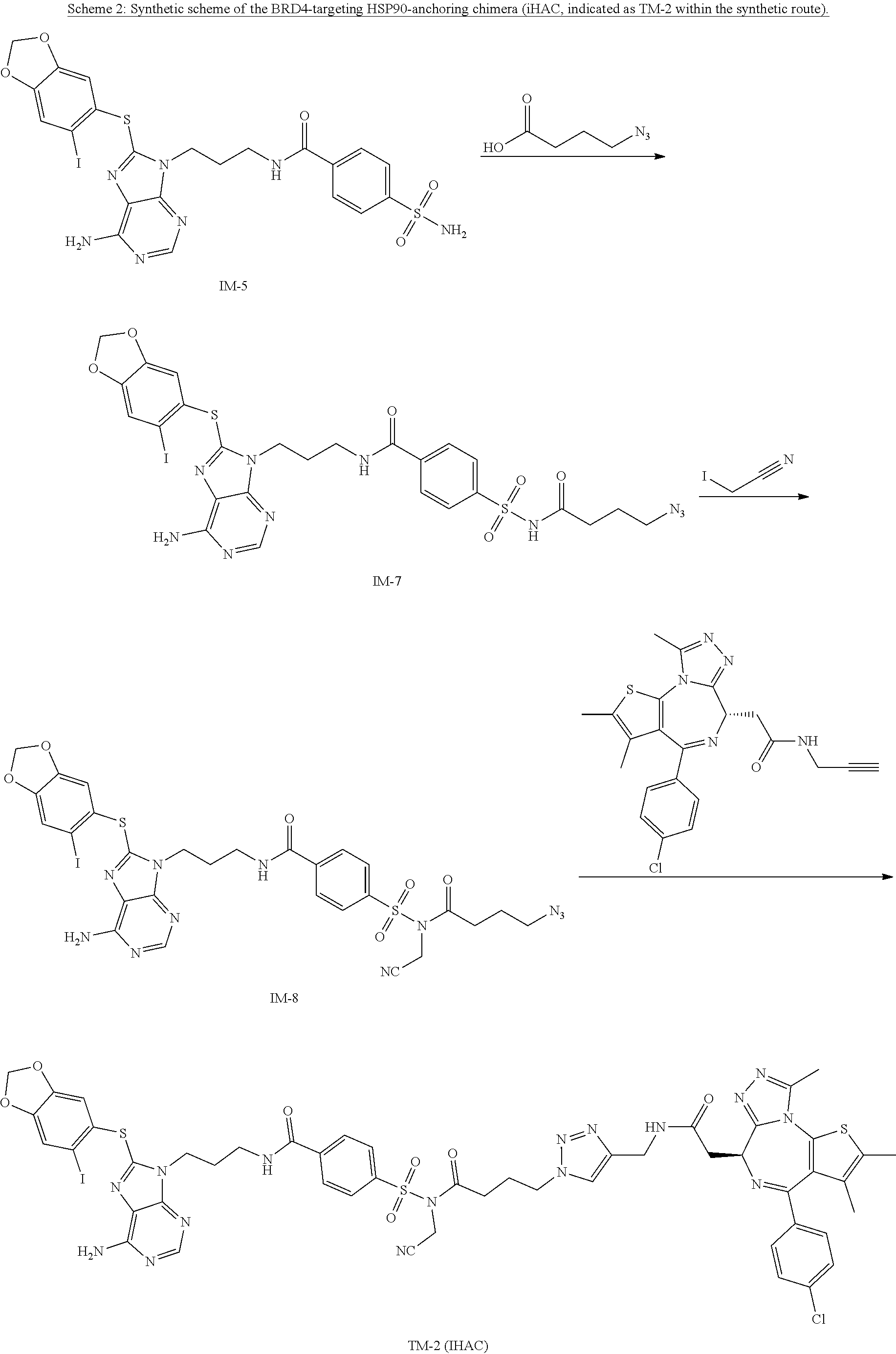

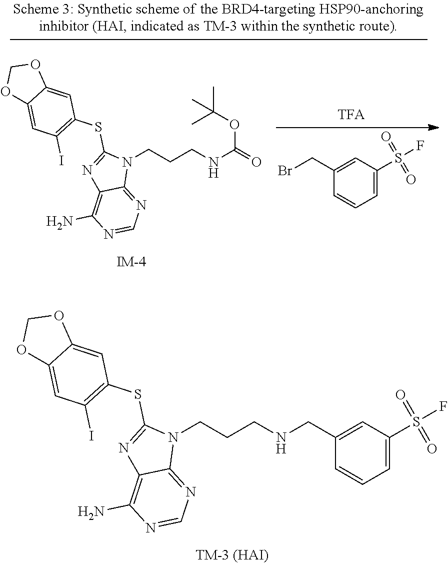

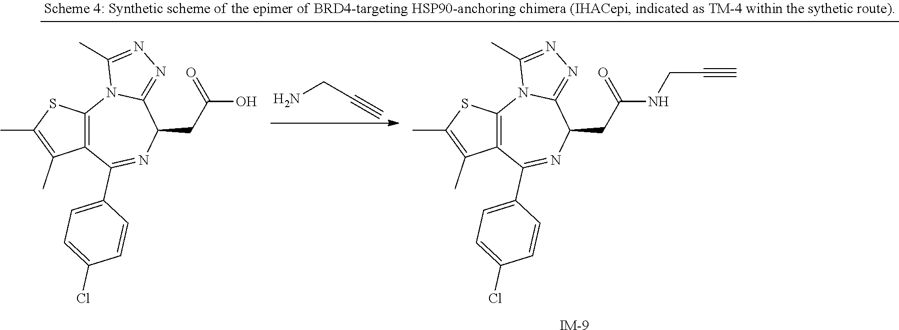

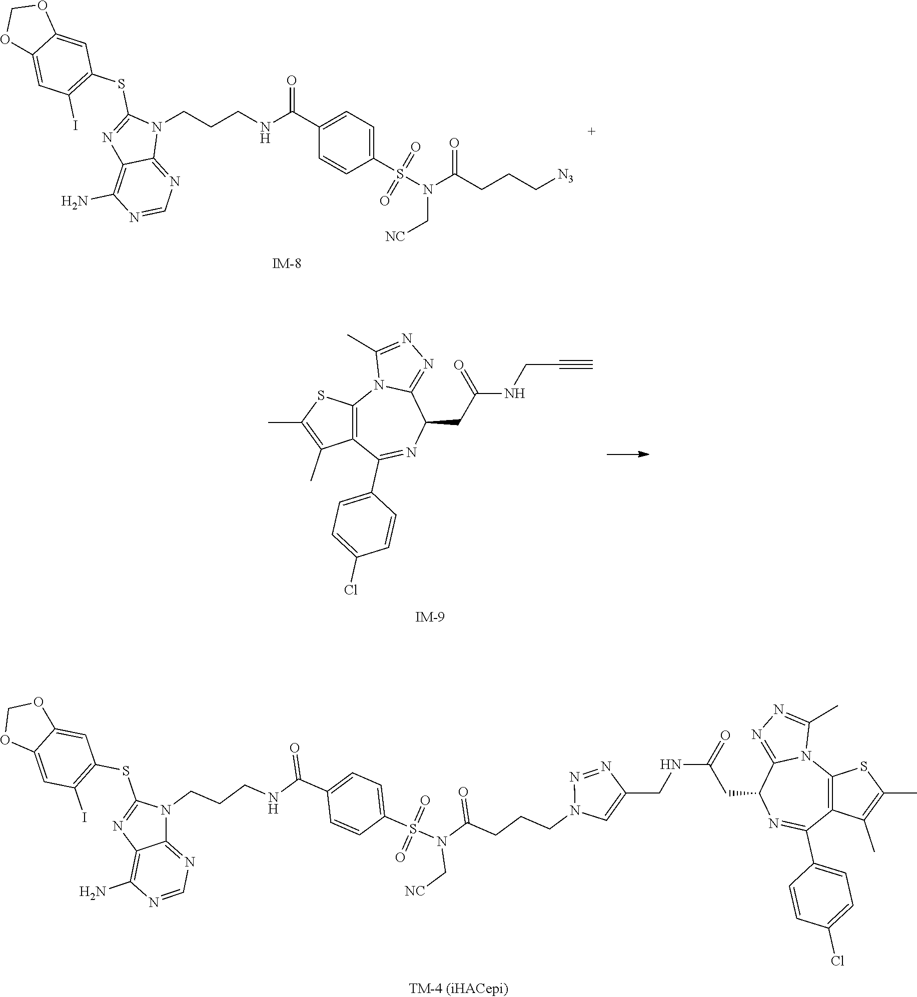

### Example 2: Activated iDePLT Packages BRD4 Ligand-Labeled HSP90 into PMPs for BRD4 Degradation in Cancer Cells

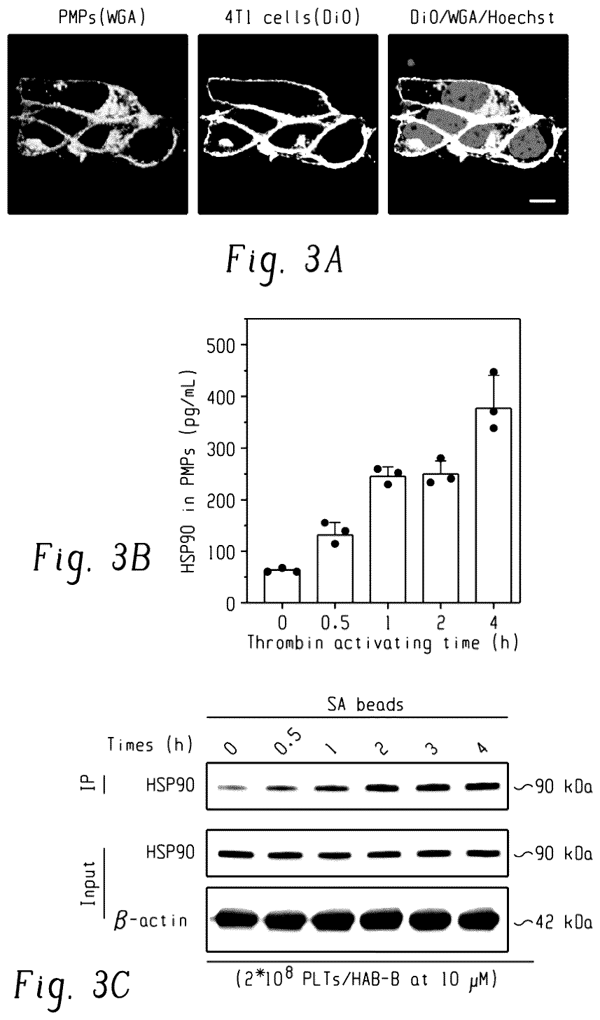

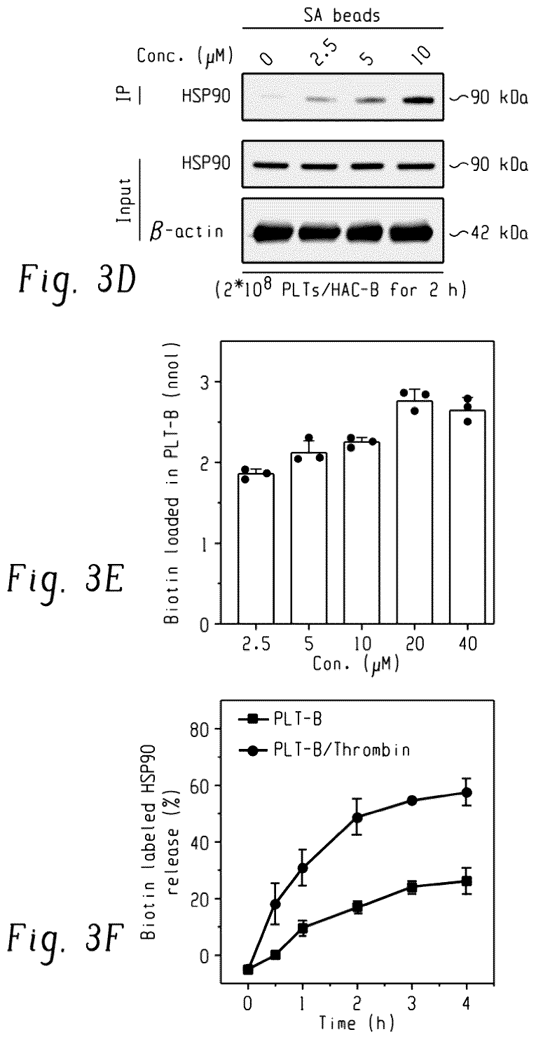

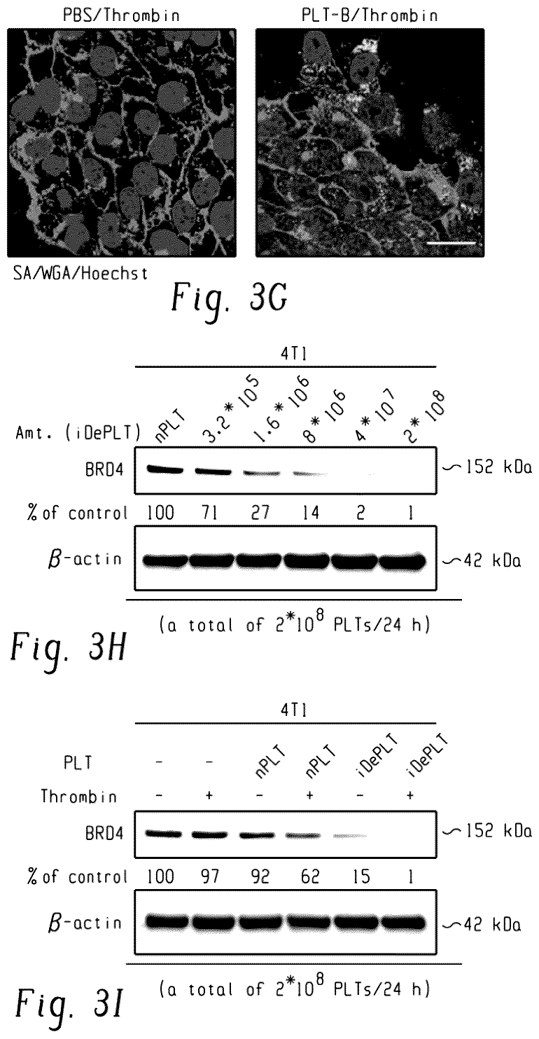

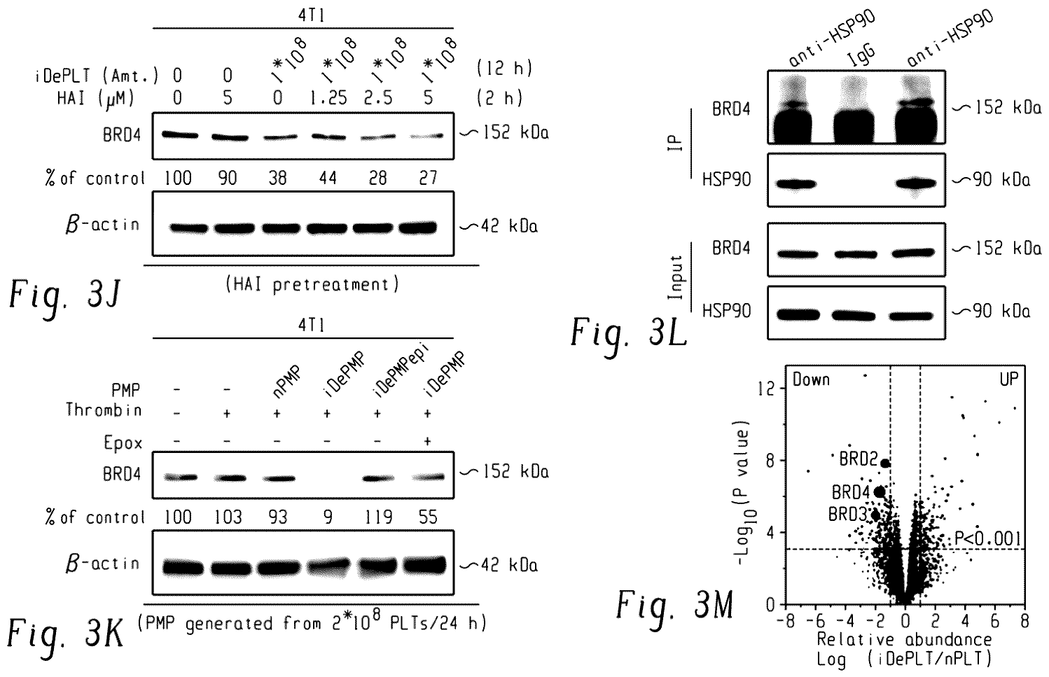

A previous study shows that activated platelets could generate abundant PMPs transporting surface-conjugated drugs to targeted cells. Thus, in this study, prior to engineering platelets with iHAC, membrane fusion between PMPs and cancer cells was observed. Fresh platelets collected from mice were labeled with rhodamine-conjugated wheat germ agglutinin (WGA), followed by the treatment with thrombin to activate platelets. The supernatant of the activated platelets, which contains PMPs, was used to incubate 4T1 cells. After staining 4T1 cells with the green membrane probe, as shown in FIG. 3A, the red signal from PMPs largely merged with the green signal of the 4T1 cell membrane, consistent with the published results that PMPs released from activated platelets could transfer biomolecules through membrane fusion. Then, based on the proof that HSP90 is expressed in both resting and activated platelets (data not shown), PMPs were isolated from the supernatant of activated platelets, and the content of HSP90 was detected in the PMPs. As more PMPs were released from activated platelets with extended activation, the total HSP90 content packaged in the released PMPs exhibited an increasing trend (FIG. 3B).

Subsequently, based on the effective concentrations of HAC-B to label HSP90 in 4T1 cells (FIG. 2B), platelets were incubated with HAC-B at 10 μM for different time points, and the platelet lysates were subjected to the treatment with the same amount of streptavidin-beads for IP assay. The immunoblot analysis exhibited that HAC-B at 10 μM could label HSP90 in platelets with biotin over time (FIG. 3C), approaching a maximum at 2 h. Also, within 2 h, HAC-B would tether HSP90 in a concentration-dependent manner (FIG. 3D). The quantification of the biotin-labeled HSP90 in platelets showed that the biotin-labeled content inside platelets reached a peak after incubation with HAC-B at 20 μM (FIG. 3E). Thus, 20 μM of HAC-B was selected to generate the engineered platelets (designated PLT-B) for the following studies. To verify the engineering method could not considerably change the physiological properties of platelets, the surface makers of the platelets incubated with HAC-B for 2 h were detected under resting and activated conditions. It was demonstrated that PLT-B showed similar expression of CD41, CD61, and CD9 as naïve platelets (nPLT) in the resting condition (data not shown). After thrombin treatment, both PLT-B and nPLT upregulated the platelet activation marker, CD62p (P-selectin). Furthermore, PLT-B could efficiently bind to collagen (data not shown), indicating the retention of collagen-binding ability. Combined with the results that both PLT-B and nPLT rapidly aggregated after activation (data not shown), it was demonstrated that the engineering procedures did not change the platelet functionality. Also, when PLT-B underwent activation, the content of biotin-labeled proteins released was increased as the thrombin treatment time extended (FIG. 3F). PLT-B was used to treat 4T1 cells, and then distribution of biotin was detected by fluorescent streptavidin. A large amount of biotin-labeled proteins appeared inside cancer cells (FIG. 3G), indicating that the labeled proteins in the platelets could be transferred into the targeted cells. Collectively, the HAC-mediated method could covalently label the HSP90 of platelets with an additional ligand without dramatic changes in physiological features, and the obtained platelets were able to transport the labeled HSP90 to targeted cells through PMP-mediated membrane fusion.

According to the efficiency of generating PLT-B with HAC-B, incubation with iHAC at 20 μM for 2 h for every 10⁸ platelets was adopted as the approach to produce the corresponding intracellular protein degradation platelet, designated iDePLT. Since HAC-B-mediated modification showed no obvious effect on the features of platelets, it is reasonable to speculate that iHAC could not induce changes in iDePLT coupled with the results that there is no BRD4 expression in a nuclear platelets (data not shown). 4T1 cells were incubated with iDePLT at different amounts for 24 h (FIG. 3H). In this case, the amount of 2×10⁸ iDePLTs is set as the maximum, and the lower doses of iDePLTs should be supplemented with nPLTs to reach the same platelet amounts as the maximum. The results demonstrated that iDePLT substantially degraded BRD4 in 4T1 cells in a dose-dependent manner. Additionally, compared to the iDePLTs without thrombin treatment, the thrombin-activated iDePLTs showed more potent degradation efficiency (FIG. 3I). This activation-controlled degradation effect endowed the iDePLT with selectivity for targeting BRD4 in the specific diseased cells. 4T1 cells exhibited a relatively stable HSP90 expression after the treatments with resting or activated iDePLT (data not shown). Next, the HAI-pretreated 4T1 cells were incubated with iDePLT and it was found that the chemical blockage against HSP90 in targeted cells failed to relieve BRD4 downregulation (FIG. 3J). This result provided proof of the dependency of iDePLT-mediated BRD4 degradation on the HSP90 transferred from iDePLT to the cancer cells. Then, iHACepi was employed to generate the control platelet of iDePLT, termed iDePLTepi. Following the thrombin treatment, the corresponding PMPs in the supernatant of iDePLT and iDePLTepi were collected, designated iDePMP and iDePMPepi, and used to treat 4T1 cells. The failure of iDePMPepi to reduce BRD4 expression suggested that iDePLT-mediated BRD4 degradation depended on direct binding to this target (FIG. 3K). Moreover, iDePMP exhibited compromised degradation potency in 4T1 cells with Epox presence, indicating that iDePLT could hijack the proteasome in targeted cells to downregulate BRD4.

Further, the co-IP experiment was performed by the anti-HSP90 antibody for the cell lysate of iDePMP-treated 4T1 cells. The immunoblot analysis showed that iDePMP treatment induced the enhanced interaction between HSP90 and BRD4 (FIG. 3L). Combined with the previous results, without being held to theory, it was hypothesized that BRD4 ligand-labeled HSP90 encapsulated in PMPs could be transferred to targeted cells and form a binary complex with BRD4, thereby initiating UPS-mediated BRD4 degradation. Finally, the label-free quantitative proteomics analysis was used to detect the changes in the abundance of whole-cell proteins in the iDePLT-treated 4T1 cells. Among the 4,511 proteins quantified, the proteins with an absolute fold-change difference over 2 were compared between iDePLT- and nPLT-treated 4T1 cells. As shown in FIG. 3M, iDePLT significantly degraded all of BRD4, BRD2, and BRD3 compared to nPLT treatment. Another 19 proteins with remarkable downregulation after iDePLT treatment exhibited a physical or functional connection with BRD4, BRD2, and BRD3 (data not shown). Collectively, it has been demonstrated that iDePLT has a strong on-target effect of degrading the BRD4 in the 4T1 cells.

### Example 3: IDePLT Inhibits Tumor Recurrence and Metastasis in Post-Surgery Mice with Primary Breast Cancer

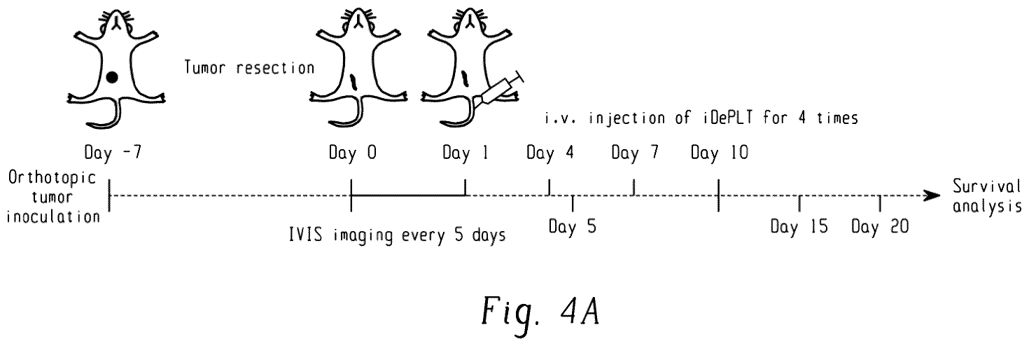

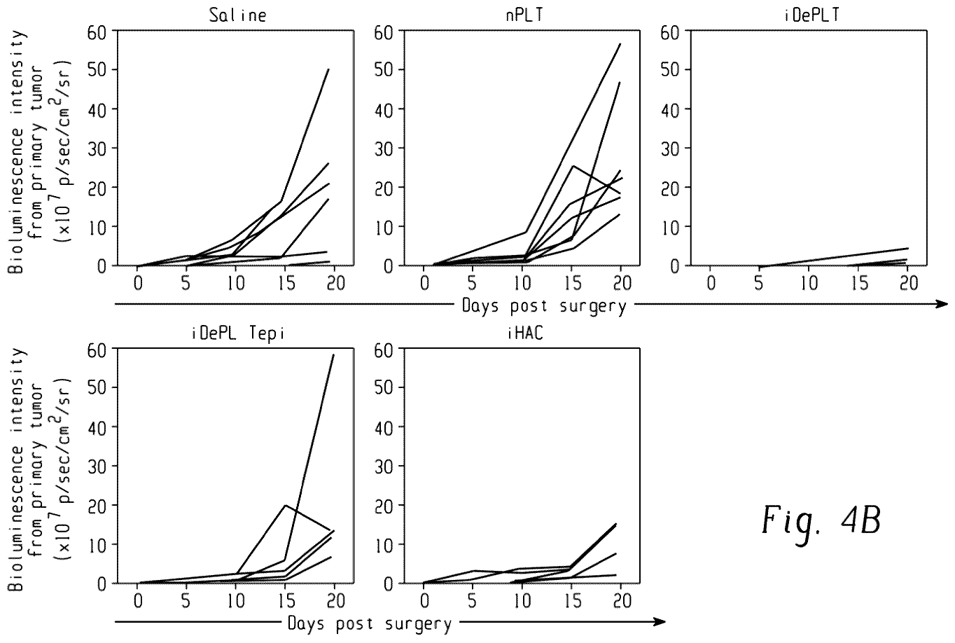

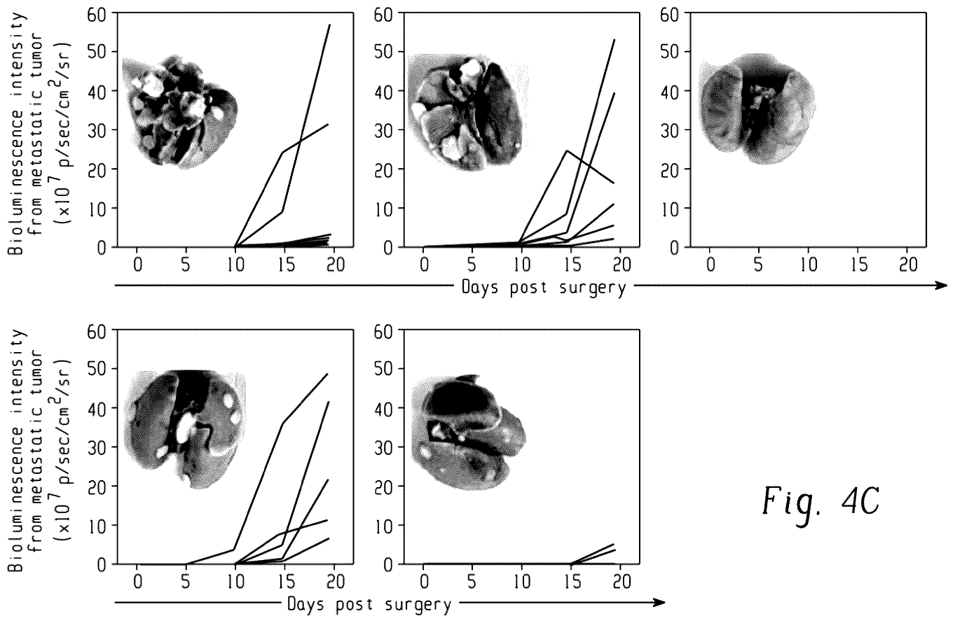

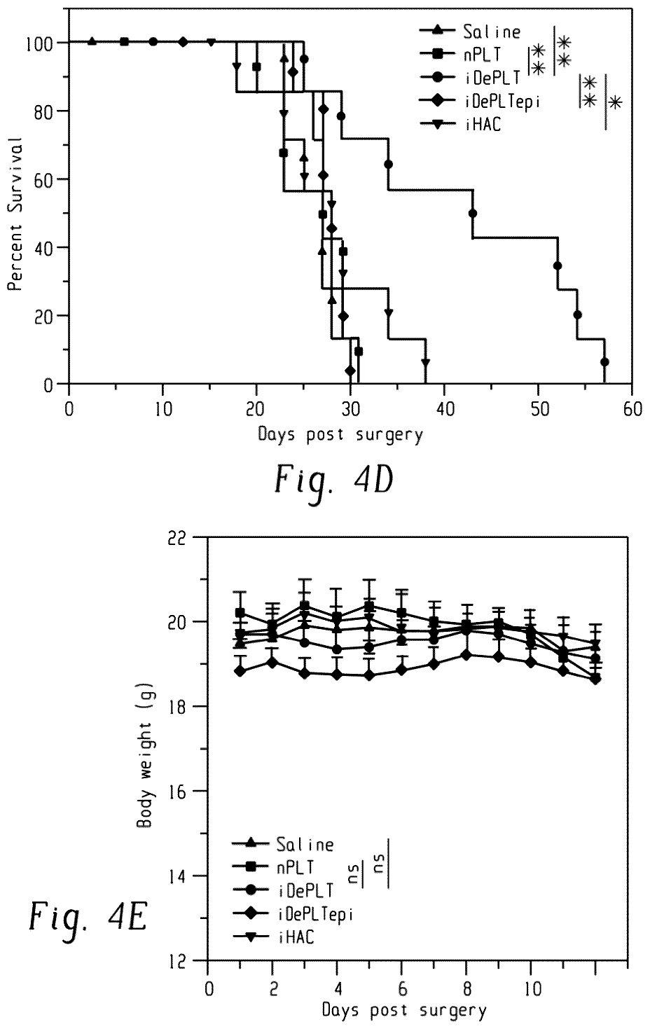

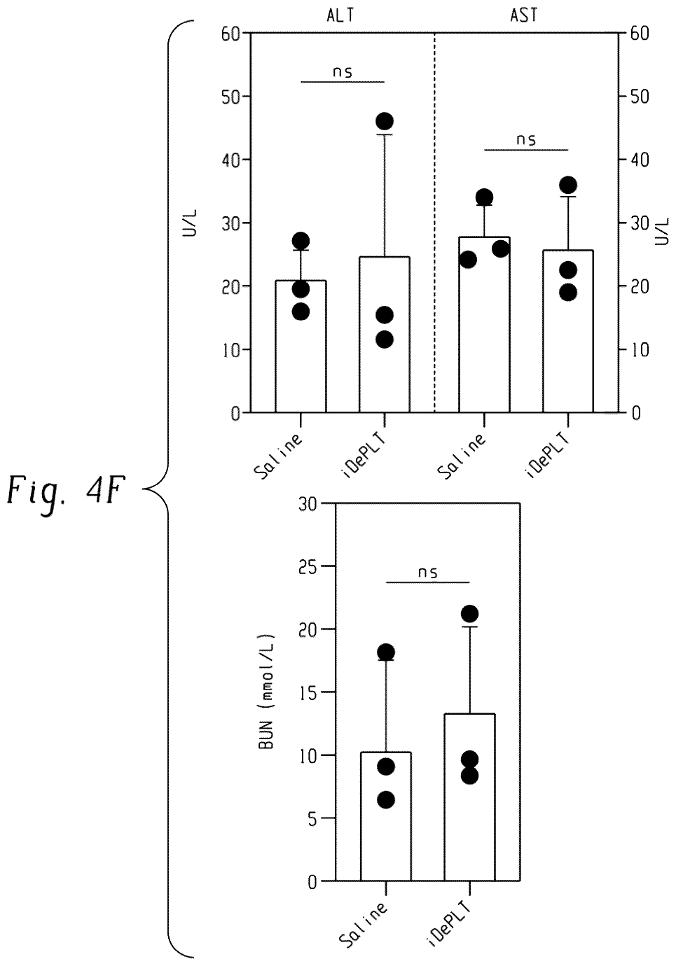

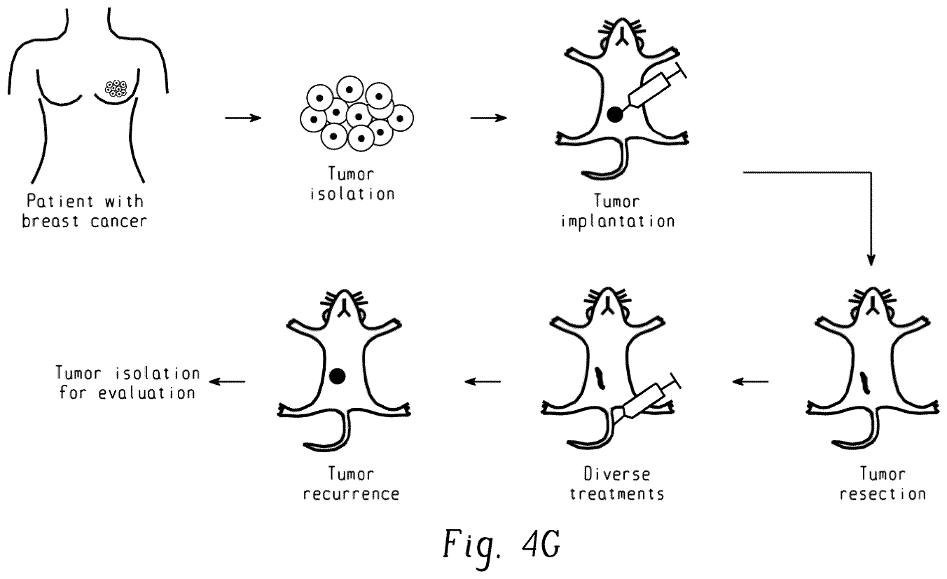

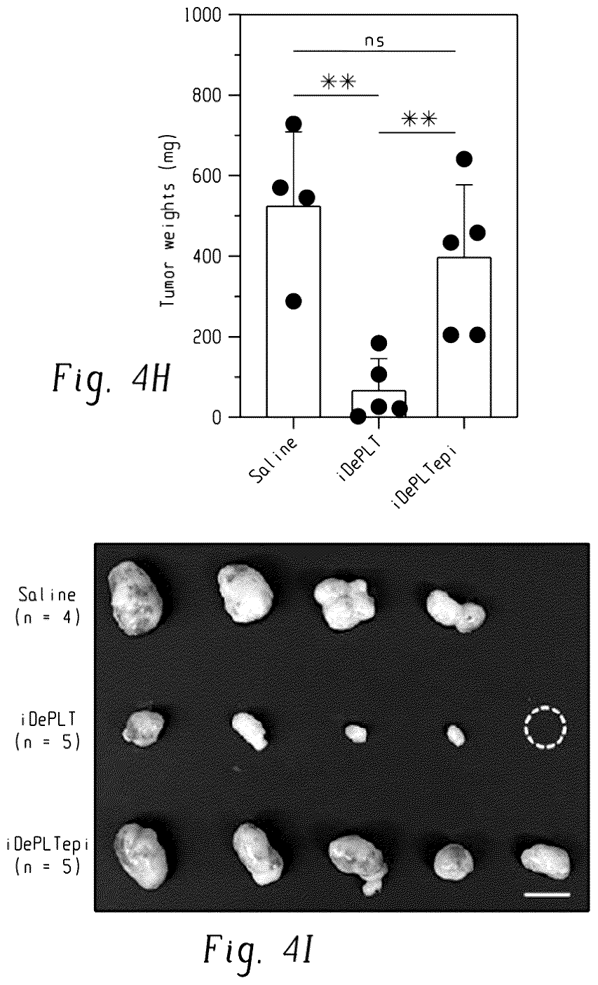

In the clinic, the TNBC has extremely poor treatment outcomes, even with multiple therapeutic approaches, including surgery, chemotherapy, and immunotherapy. Notably, surgery is the major treatment option for TNBC clinically, while the high frequency of TNBC recurrence could lead to significant morbidity and mortality. To test the in vivo treatment efficacy of iDePLT against TNBC recurrence, a post-surgical TNBC mouse model was established by injecting the luciferase-expressing 4T1 (4T1-Luc) cells into the fourth mammary gland fat pads of BALB/c mice. After the tumor reached about 100 mm³, surgical resection was performed to totally remove the visible tumor tissues under a microscope. The postoperative mice were randomly divided into five groups (n=7), which accepted the treatments with saline, nPLT, iDePLT, and iDePLTepi through intravenous (i.v.) injection every third day for 4 times (FIG. 4A). And the mice treated with intraperitoneal (i.p.) injection of free iHAC (5 mg/kg) served as a control group. The tumor relapse was evaluated by detecting the bioluminescence signals from 4T1-Luc cells in the surgical areas. In previous studies, it was substantiated that the i.v. injection of engineered platelets could actively and effectively home to the post-surgical TNBC site and locally release therapeutic payloads for eliminating residual TNBC cells. Without being held to theory, it was hypothesized that iDePLT would also preferentially accumulate at the cancer site and transfer BRD4 ligand-tethered HSP90 to the targeted cells by generating PMPs after activation, realizing controllable TPD in the residual malignant cells.

The imaging results showed that there were negligible bioluminescence signals around the operative sites of all these mice on day 0 after surgery (FIG. 4B). Along with the administration, mice treated with saline and nPLT suffered from relapse and exhibited severe lung metastasis on day 15 (FIG. 4B, 4C). In contrast, iDePLT-treated mice showed slight tumor recurrence without obvious metastasis during the imaging period. Compared to iDePLT, there was almost no therapeutic efficacy with iDePLTepi injections, and strong bioluminescence signals were detected in surgical and lung areas from day 15. Additionally, free iHAC showed some anti-relapse effect during the dosing time, but the mice finally showed substantially recurrent tumors on day 20. Due to the potent inhibitory effect on recurrence and metastasis, iDePLT significantly prolonged the survival of post-surgery mice compared to the mice treated with saline, nPLT, iDePLTepi, and free iHAC (FIG. 4D). The unnoticeable treatment efficacy from iDePLTepi treatments demonstrated that iDePLT realized suppression of tumor recurrence and metastasis through targeting BRD4. Within the dosing period, the body weights of these post-surgery mice were recorded every day, and no significant changes were observed in mice injected with iDePLT compared to saline and nPLT (FIG. 4E), revealing the good biosafety of iDePLT administration. Further, the biosafety profiles of iDePLT were systematically assessed through histopathological analysis. The hematoxylin and eosin (H & E) staining indicated that there is no obvious pathological damage in all the main organs, including the heart, liver, spleen, lung, and kidney (data not shown). The blood biochemical and hematological examination were also performed for iDePLT-treated mice. The levels of alanine transaminase (ALT) and aspartate transferase (AST) in the serum of treated mice did not show significant changes compared to mice in the saline treatment group, indicating negligible damage to the liver caused by iDePLT treatment (FIG. 4F). Since the blood urea nitrogen (BUN) index maintained similar concentrations between the mice treated with saline and iDePLT, iDePLT also did not induce injury in the kidney. Moreover, all the hematological indexes are generally stable after iDePLT treatments. (data not shown). These biosafety profiles demonstrated that iDePLT possessed favorable biocompatibility.

The encouraging anti-relapse outcomes of iDePLT prompted testing the treatment efficacy with the patient-derived xenograft (PDX) model. An orthotopic breast cancer was first established with patient specimens on NSG mice (FIG. 4G). After the tumor sizes reached about 100 mm³, the visible tumor tissues were removed by resection under a microscope. The post-surgical NSG mice were randomly separated into three groups for the treatments with saline (n=4), iDePLT (n=5), and iDePLTepi (n=5) through i.v. injection every third day for 4 times. The tumor tissues were isolated after two weeks of the last treatments, and the tumors collected from the iDePLT-treated mice showed the lowest weights and smallest sizes (FIGS. 4H and 4I), with one almost disappearing, indicating the robust potency of iDePLT to inhibit tumor recurrence in NSG mice. Also, during the dosing period, no obvious body weight changes were observed in mice from the iDePLT treatment group compared to the mice in the other two groups (data not shown). Collectively, the in vivo evaluation demonstrated that iDePLT could serve as a promising therapeutic for the postoperative treatment of breast cancer.

### Example 4: EDePLT Releases PD-L1 Ligand-Labeled Extracellular HSP90 to Degrade PD-L1 On Cancer Cells by Repurposing Lysosome Machinery

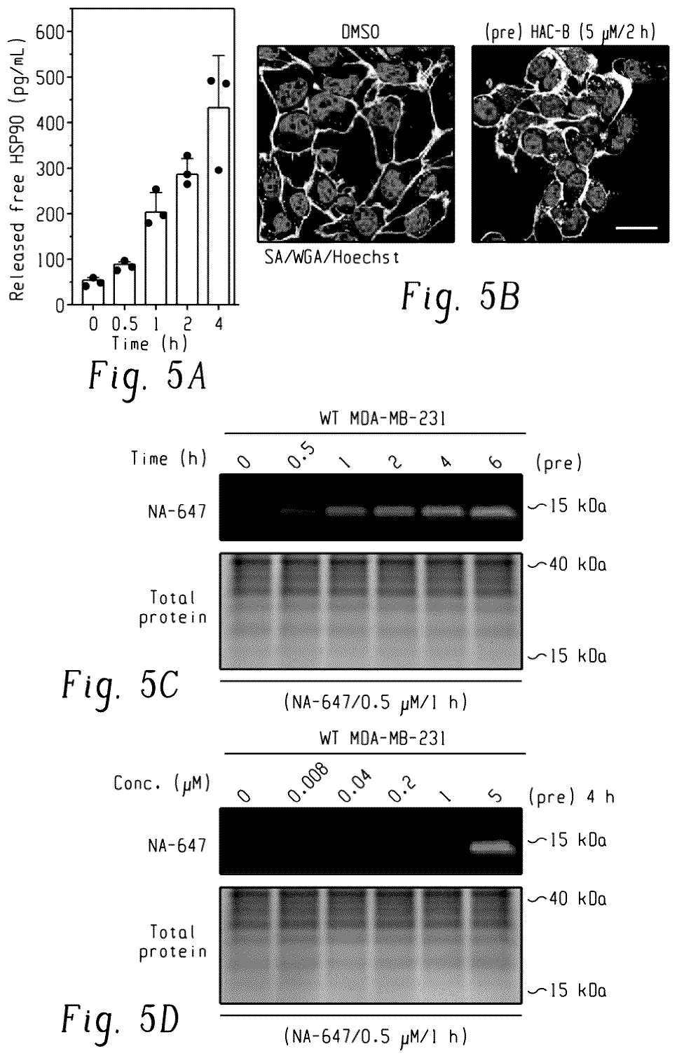

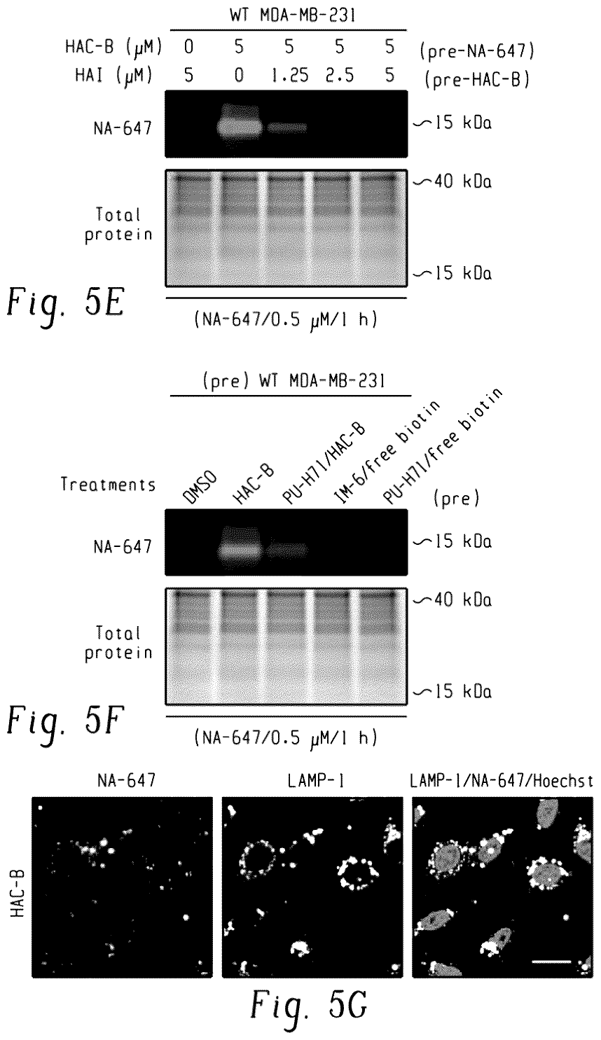

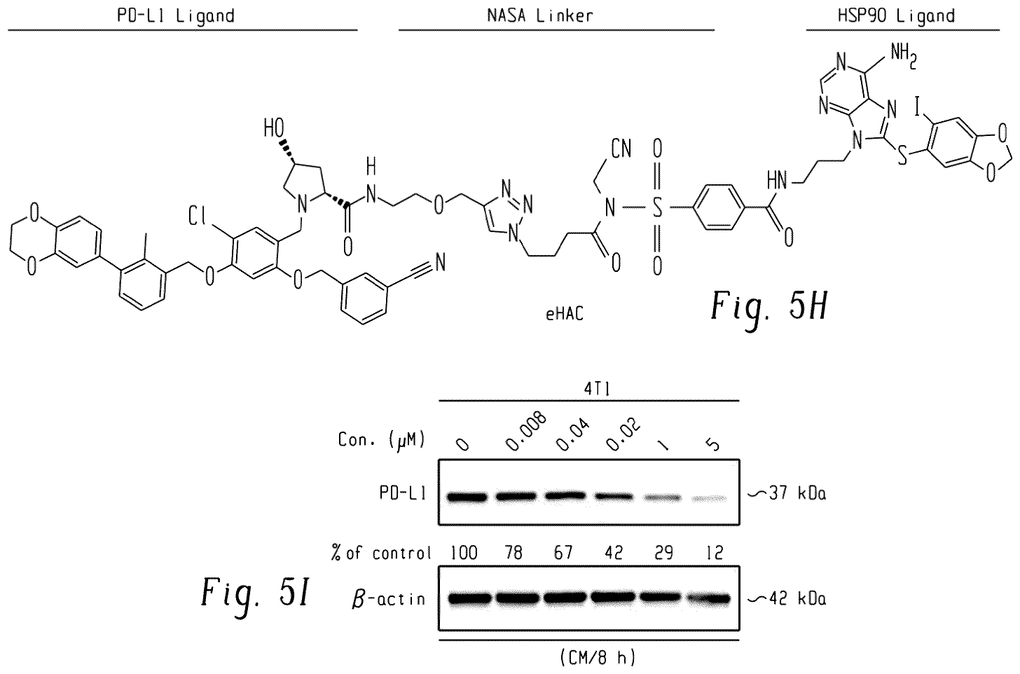

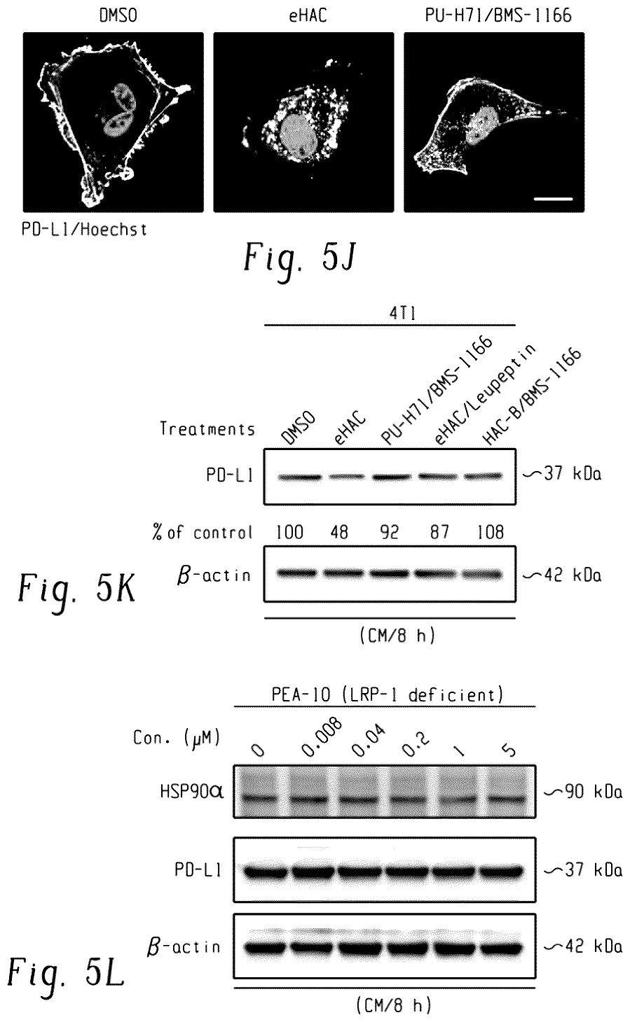

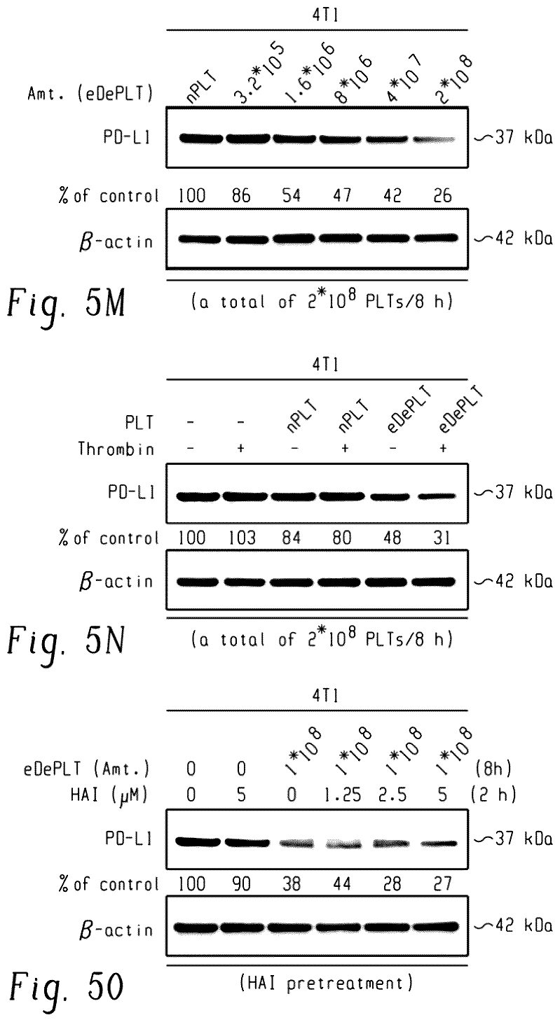

When incubated 4T1 cells were incubated with activated PLT-B, a portion of biotin-labeled proteins appeared inside cancer cells, and the other part of these proteins was stranded on the membrane of 4T1 cells (data not shown). Platelets have been reported to release free HSP90 to the extracellular space after activation, and the presence of free HSP90 in was detected in the supernatant collected from the activated platelets (FIG. 5A). Without being held to theory, it was speculated that these signals were caused by the free extracellular HSP90. Various cells, including cancer cells, have been reported to actively secret HSP90 to the external environment. Also, recycling the HSP90 from the extracellular space into the intracellular compartment is a part of the cellar regulation of this secreted protein. Hence, HAC-B was used to label the HSP90 and then streptavidin-FITC was adopted to track its distribution. 4T1 cells were pretreated with HAC-B for 2 h and streptavidin-FITC was added to the medium. Compared to the cells without pretreatment, much stronger green fluorescence signals appeared in the cytoplasm or on the membrane of cancer cells, consistent with 4T1 cells cotreated with HAC-B and streptavidin-FITC (FIG. 5B and data not shown). The results indicated that HSP90 existed in the extracellular space of 4T1 cells, and HAC-B could hijack the extracellular HSP90 to shuttle streptavidin into the intracellular region. To quantify the HAC-B-driven uptake of extracellular proteins, cancer cells were pretreated with HAC-B for 2 h, and then NeutrAvidin™-647 (NA-647) was added to the medium. After 1-h incubation, the in-gel fluorescence was analyzed to measure the internalized NA-647. In WT MDA-MB-231 cells, HAC-B was confirmed to enhance the internalization of extracellular NA-647 in a time and concentration-dependent manner (FIG. 5C and data not shown). However, when 4T1 cells were cotreated with HAC-B and NA-647, all the concentrations of HAC-B failed to internalize NA-647 in a large quantity within 2 h (data not shown). After optimization of the pretreatment procedure, it was verified that HAC-B pretreatment at 5 μM for 4 h could induce cancer cells to substantially internalize NA-647 (FIG. 5D). Furthermore, WT MDA-MB-231 incubated with HAI prior to HAC-B pretreatment showed a largely compromised NA-647 uptake (FIG. 5E), which was closely associated with the concentration of HAI. At the same concentration of HAC-B, there was just a slight NA-647 internalization in WT MDA-MB-231 after the pretreatments with the combinations of PU-H71 plus HAC-B, IM-6 (the synthetic intermediate) plus free biotin, and PU-H71 plus free biotin (FIG. 5F). These results highlighted the importance of HAC-B simultaneously binding to HSP90 and NA-647 during the internalization process. According to published studies, the internalized extracellular HSP90 would undergo the endosome pathway to lysosome. Thus, the distribution of internalized NA-647 in WT MDA-MB-231 cells was detected, and the red signals mainly overlapped with the lysosome maker, LAMP1 (FIG. 5G and data not shown). Coupled with the features of HAC-mediated covalent labeling, it is reasonable to claim that extracellular POI ligand-labeled HSP90 in outer space would drive the internalization of the corresponding proteins in the targeted cells and transport them to the lysosome.

The results with HAC-B and NA647 encouraged exploration of the potential of extracellular HSP90 as an effector protein to initiate the degradation of the POIs in outer space via lysosome machinery. The programmed death-ligand 1 (PD-L1) on the membrane of cancer cells was selected as the extracellular POI, and the relevant HAC was constructed based on the PD-L1 inhibitor, BMS-1166 (FIG. 5H and Scheme 5). According to the PD-L1 expression among different cancer cells, HSP90α-deficiency had little effect on the PD-L1 levels in MDA-MB-231 cells (FIG. S20A). When the obtained HAC targeting PD-L1, designated eHAC, was dispersed in the blank medium (BM) and the 4T1 cells treated, the eHAC just slightly reduced the PD-L1 expression within 12 h. Next, the supernatant from the culturing medium of 4T1 cells was collected after centrifugation and filtration, termed the conditioned medium (CM), which was then used to dilute eHAC. The results showed that eHAC suspended in CM could efficiently downregulate PD-L1 levels at the same treatment time points (FIG. 5I and data not shown). This difference also appeared in WT MDA-MB-231 cells after eHAC treatments (FIG. S20C), indicating the involvement of free extracellular HSP90 released from the parent cells in the eHAC-mediated PD-L1 degradation. Since eHAC reduced PD-L1 expression in a time- and concentration-dependent manner, the PD-L1 distribution was then detected in eHAC-treated cancer cells. Compared to WT MDA-MB-231 cells after the treatment with the combination of PU-H71 and BMS-1166, the PD-L1 rapidly aggregated into punctate structures and moved towards cytosol upon 2-h eHAC treatment (FIG. 5J). Combined with the results of PD-L1 downregulation and NA-647 internalization (FIG. 5G), it was proposed that internalized PD-L1 would be finally transported to the lysosome for degradation, which is confirmed by the rescue effect on PD-L1 expression in the presence of lysosome degradation inhibitor, leupeptin, during eHAC treatment (FIG. 5K). The powerful degradation potency of eHAC was considerably compromised in HSP90α-KO MDA-MB-231 (data not shown), presenting in an HSP90-dependent downregulation manner. Different from eHAC treatment, negligible PD-L1 degradation was achieved in the 4T1 cells after the treatments with the combinations of PU-H71 plus BMS-1166 and HAC-B plus BMS-1166 (FIG. 5K), indicating the necessity of eHAC simultaneously binding to HSP90 and PD-L1 during the transportation to the lysosome. It has been reported that extracellular HSP90 binds to the surface receptor, low-density lipoprotein receptor-related protein 1 (LRP-1), and LRP-1 is involved in the receptor-mediated endosome-lysosome pathway. The PD-L1 expression was tested in an LRP-1-deficient cell line, PEA-10, after eHAC treatment. Although this cell line has a stable HSP90α level, eHAC failed to induce obvious PD-L1 degradation (FIG. 5L). This result provided proof that eHAC facilitated extracellular HSP90 to capture PD-L1 and transport the latter to the lysosome via LRP-1-mediated endocytosis.

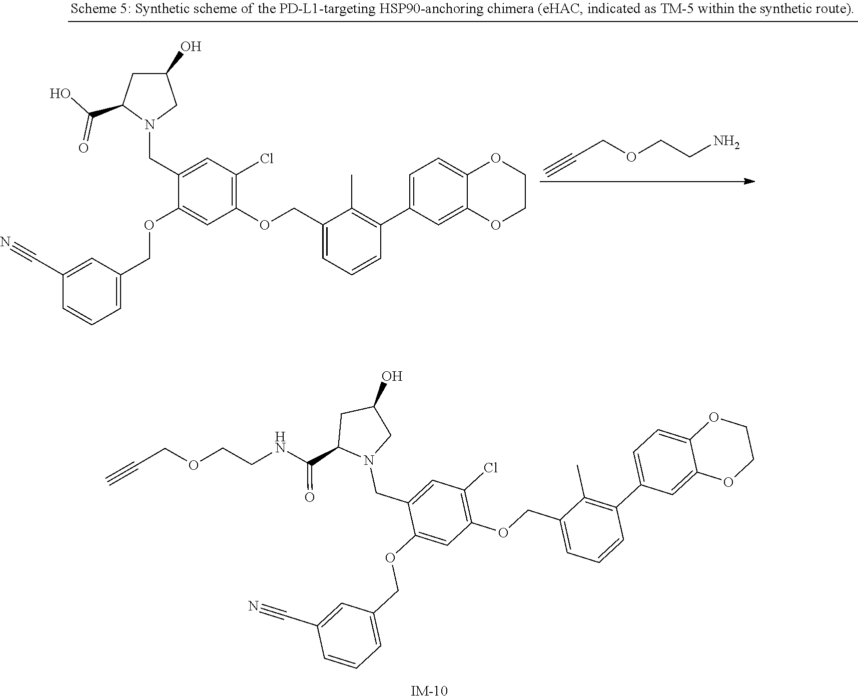

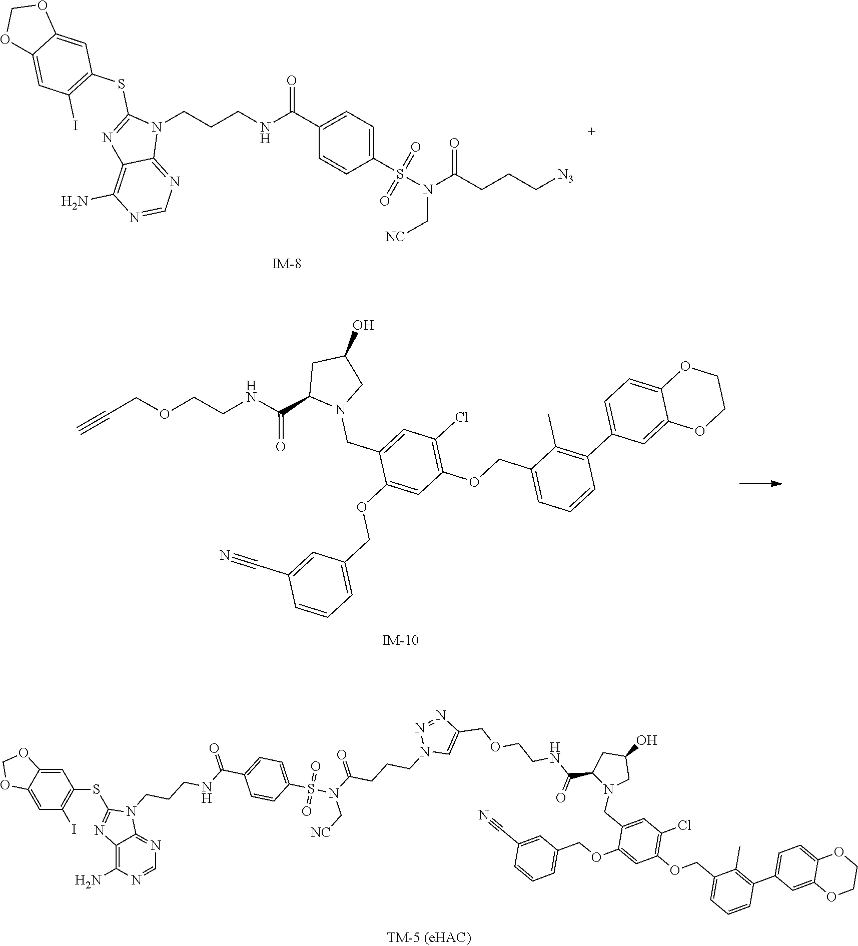

Subsequently, we eHAC was used to incubate the platelets to generate the corresponding engineered platelets targeting PD-L1, designated eDePLT. After coincubation with eDePLT, the PD-L1 expression in 4T1 cells was substantially reduced, which is closely associated with the number of eDePLT (FIG. 5M). Also, the downregulation of PD-L1 expression in 4T1 cells exhibited a time-dependent response to eDePLT treatment (data not shown). In comparison to the eDePLTs without thrombin addition, the activated eDePLT could more efficiently degrade PD-L1 (FIG. 5N), revealing the controllability and selectivity of eDePLT-mediated PD-L1 degradation. Next, HAI was employed to chemically block the HSP90 in 4T1 cells and subjected the pretreated cells to eDePLT treatment. The results showed that the chemical blockage against HSP90 in 4T1 cells failed to rescue the PD-L1 degradation (FIG. 5O), suggesting that eDePLT utilized the HSP90 from platelets to downregulate PD-L1 in targeted cells. Finally, the supernatant from the activated eDePLT was collected to treat 4T1 cells with or without the presence of leupeptin. It was found that the extracellular content released from activated eDePLT potently reduced PD-L1 levels in targeted cells through a lysosome-dependent machinery (data not shown). Collectively, upon the activation, eDePLT could release PD-L1 ligand-labeled HSP90 to the extracellular space, which was able to capture the PD-L1 on the membrane surface of the targeted cells and then redirect PD-L1 to lysosome through the LRP-1-mediated endocytosis.

### Example 5: EDePLT Degrades PD-L1 and Elicits Potent Antitumor Immune Response Against TNBC Recurrence and Metastasis in a Post-Surgical Mouse Model

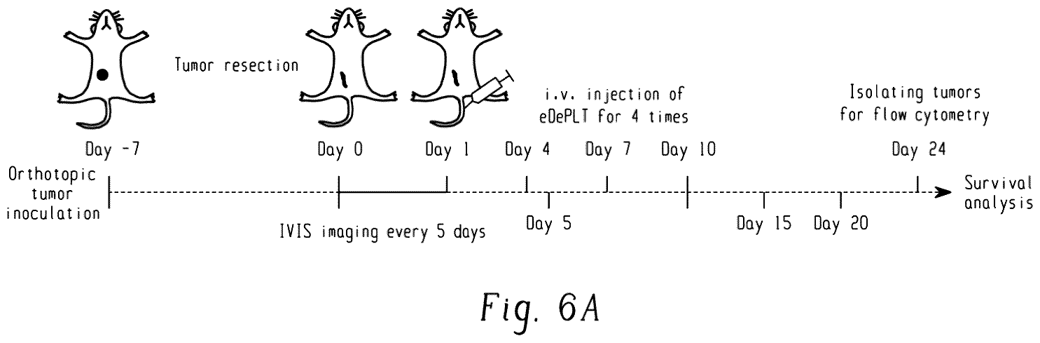

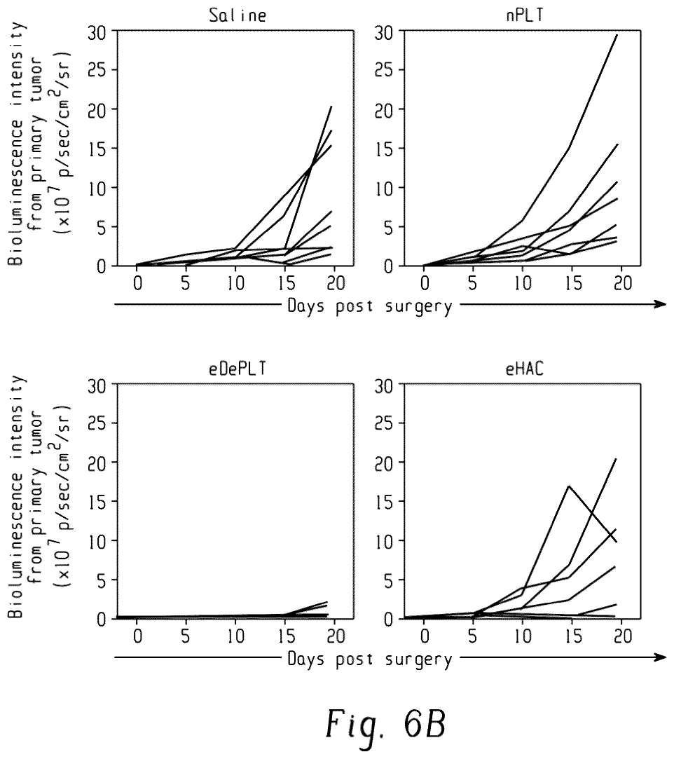

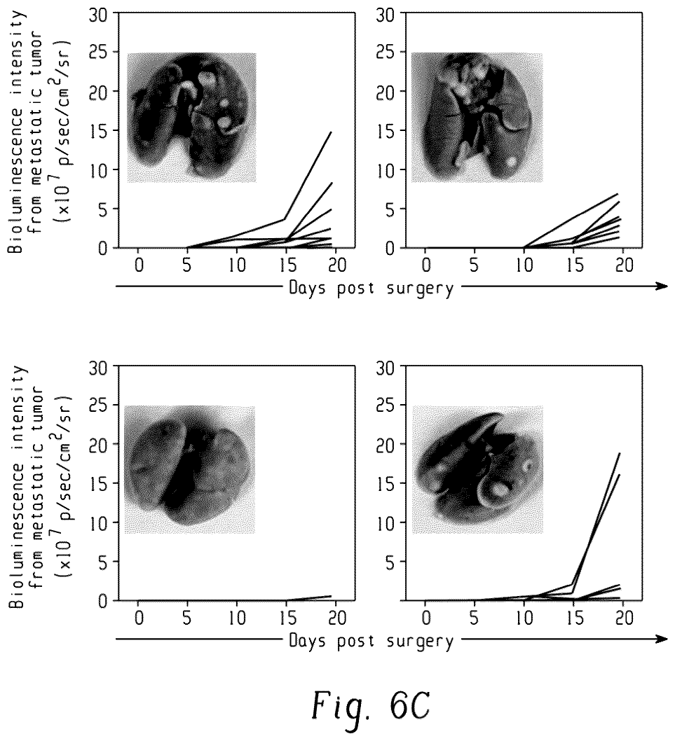

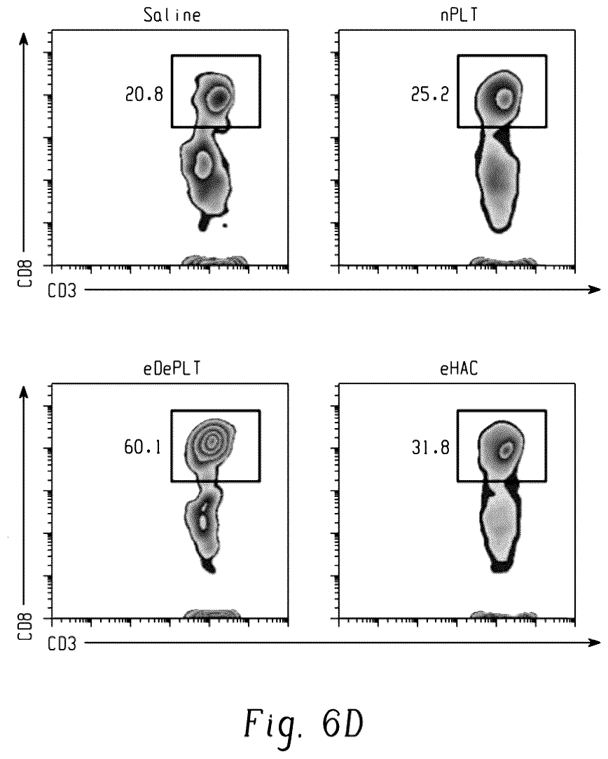

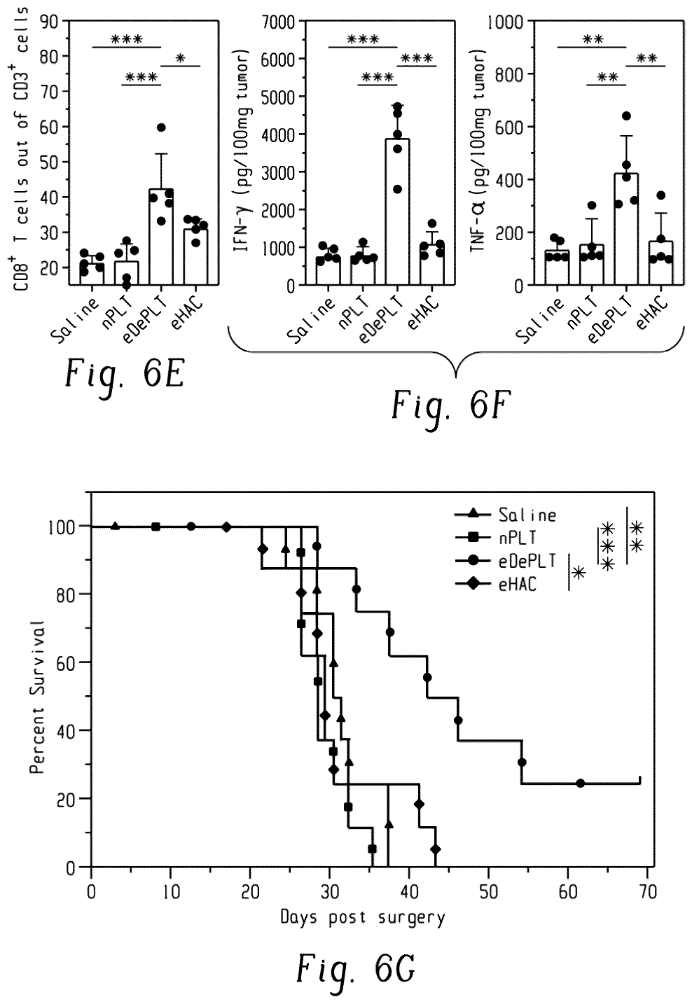

It has been reported that blockage to the PD-L1 on the cancer cells is beneficial to activating the anticancer immune response. Without being held to theory, it was expected that eDePLT would degrade the PD-L1 on the residual malignant cells within the surgical cavity and improve the postoperative treatment of TNBC. The murine post-surgical model was established with 4T1-Luc cells as aforementioned. The postoperative mice, randomly divided into four groups (n=8), were i.v. injected with saline, nPLT, and eDePLT every third day for 4 times. The mice injected (i.p.) with free eHAC (5 mg/kg) served as the control group (FIG. 6A). The tumor recurrence was detected through bioluminescence signals from 4T1-Luc cells around the surgical areas.

Based on the imaging results, all the post-surgical mice showed negligible bioluminescence signals at the resection sites on day 0 after the operation (data not shown). As the treatments continued, mice in the saline- and nPLT-treated groups rapidly suffered from tumor recurrence on day 15 post-surgery (FIG. 6B). The same severe relapse was also found in the mice treated with free eHAC. On the contrary, the eDePLT administration efficiently suppressed the tumor recurrence course, and there were even four mice free of relapse during the imaging period. Consistent with the tumor recurrence at the primary sites, some of the mice treated with saline, nPLT, and free iHAC started to exhibit obvious lung metastasis from day 15 after surgery, and all the eDePLT-injected mice had no detectable bioluminescence signal from the lung area within 20 days post-surgery (FIG. 6C). After two weeks of the last eDePLT treatment, the tumor-infiltrating lymphocytes within the recurrent tumors were analyzed by flow cytometry (FIG. 6D). Compared to saline and nPLT, eDePLT treatment exhibited a powerful enhancing effect on the infiltration of CD3⁺CD8⁺ T cells to the tumor tissues, thereby increasing the percentage of these effector cells 2.0 and 1.9-fold, respectively (FIG. 6E). Even though the treatments with free eHAC also slightly elevated the ratio of CD3⁺CD8⁺ T cells, the improvement was significantly lower than that caused by eDePLT. With the enhancement of the infiltrated effector T cells, the tumor tissues after eDePLT treatment showed significantly higher levels of immune-associated cytokines, including IFN-γ and TNF-α, than the other three groups (FIG. 6F). These results demonstrated that eDePLT administration activated the anticancer immune response of post-surgical mice and facilitated the infiltration of effector T cells to the resection area. Benefitting from the strengthened immune activation, the post-surgical mice from the eDePLT treatment group showed a significantly prolonged survival (FIG. 6G), and two mice achieved complete remission of tumor recurrence and metastasis on day 70 after surgery with almost no bioluminescence signals in their surgical and lung areas.

Additionally, the body weight changes of the postoperative mice were monitored during the dosing course. No marked variation was observed in the mice treated with eDePLT in comparison with the mice injected with saline and nPLT, indicating the favorable systematic biosafety of eDePLT (data not shown). The histopathological analysis was performed to observe the appearance of the main organs of the eDePLT-treated mice, including the heart, liver, spleen, lung, and kidney. Compared to the saline treatment, there are no visible morphological changes in all of the organs from eDePLT-treated mice (data not shown). After one week of the last eDePLT treatment, the blood was collected and subjected to a hematological examination. All the indexes are generally consistent with the blood from saline-treated mice (data not shown). These satisfactory biosafety profiles rendered eDePLT a biocompatible therapeutic for in vivo applications.

### DISCUSSION

Recent breakthroughs in the TPD field, benefiting from continuously updating and iterating approaches for structure optimization and chemical modification, have proved the advantages of protein degradation over binding-mediated activity blockage, thereby identifying broad application prospects of the emerging TPD technologies. However, these inspiring chimera-based pharmacological mechanisms frequently suffer from some intrinsic limitations, both structurally and functionally, which would be largely exacerbated as approaching the in vivo administration. Unlike the conventional design ideas of chimeric structures, described herein is an approach to achieve selective and effective protein degradation in cells within the lesion microenvironment. The physiological properties and pathological functionalities of platelets were maximized to develop a platelet-based integrative TPD platform through a facile chemical engineering approach, which could utilize the UPS machinery in PMP-attached cells to degrade intracellular proteins or redirect extracellular proteins to the lysosome in the targeted cells. This protein degradation strategy is a platform based on endogenous cells, specifically platelets, harboring superior biocompatibility. By grafting the TPD rationale to the platelets, this protein degrader is not only exempted from the shortcomings of physicochemical and pharmacokinetic features caused by chimeric structures, but also holds the potential to selectively accumulate at the lesion, controllably degrade the targeted proteins in the diseased cells. More importantly, different from chimeras that degrade target proteins through the effector within the same source cell, the designed proteolytic platelets achieve degradation through their self-delivered effector proteins, endowing this technology with largely extended application areas without considering the biodistribution of the effector proteins. As the role of extracellular HSP90 as an effector for lysosome-mediated TPD machinery was explored and validated in this study, the UPS-mediated TPD for intracellular protein and lysosome-based TPD for extracellular targets into the platelet-based platform extends application scenarios of TPD technologies. A new TPD platform was developed through engineering method development, mechanism validation, and final treatment efficacy verifications.

The de novo designed TPD strategy establishes an integrative platelet-based TPD platform for selective accumulation and controllable intracellular/extracellular protein degradation in vivo.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. An engineered platelet comprising a platelet cell or platelet-derived microparticle loaded with an HSP90 anchoring chimera comprising a protein of interest (POI) ligand covalently linked to a heat shock protein 90 (HSP90).

2. The engineered platelet of claim 1, wherein the platelet cell or platelet-derived microparticle is of human origin.

3. The engineered platelet of claim 1, wherein the platelet-derived microparticle has a diameter from 100 to 1000 nm.

4. The engineered platelet of claim 1, wherein the POI is an intracellular protein selected from the following table:

| | |
|---|---|
| ESR1 | Estrogen receptor |
| AR | Androgen receptor |
| BTK | Tyrosine-protein kinase BTK |
| IRAK4 | Interleukin-1 receptor-associated kinase 4 |
| EGFR | Epidermal growth factor receptor |
| MET | Hepatocyte growth factor receptor |
| KIT | Mast/stem cell growth factor receptor Kit |
| EPHA2 | Ephrin type-A receptor 2 |
| PDE4D | cAMP-specific 3′,5′-cyclic phosphodiesterase 4D |
| SRC | Proto-oncogene tyrosine-protein kinase Src |
| BRAF | Serine/threonine-protein kinase B-raf |
| FGFR2 | Fibroblast growth factor receptor 2 |
| FGFR1 | Fibroblast growth factor receptor 1 |
| LYN | Tyrosine-protein kinase Lyn |
| ITK | Tyrosine-protein kinase ITK/TSK |
| PARP1 | Poly [ADP-ribose] polymerase 1 |
| HDAC2 | Histone deacetylase 2 |
| HDAC3 | Histone deacetylase 3 |
| JAK1 | Tyrosine-protein kinase JAK1 |
| BCL2 | Apoptosis regulator Bcl-2 |
| HDAC6 | Histone deacetylase 6 |
| CRBN | Protein cereblon |
| EPHB2 | Ephrin type-B receptor 2 |
| BLK | Tyrosine-protein kinase Blk |
| HDAC1 | Histone deacetylase 1 |
| IGF1R | Insulin-like growth factor 1 receptor |
| TGFBR1 | TGF-beta receptor type-1 |
| AKT2 | RAC-beta serine/threonine-protein kinase |
| AKT1 | RAC-alpha serine/threonine-protein kinase |
| PTK2 | Focal adhesion kinase 1 |
| MAPK1 | Mitogen-activated protein kinase 1 |
| MAPK14 | Mitogen-activated protein kinase 14 |
| CDK9 | Cyclin-dependent kinase 9 |
| MCL1 | Induced myeloid leukemia cell differentiation protein Mcl-1 |
| BRD4 | Bromodomain-containing protein 4 |
| BRD3 | Bromodomain-containing protein 3 |
| CDK13 | Cyclin-dependent kinase 13 |
| BCL2L1 | Bcl-2-like protein 1 |
| CDK12 | Cyclin-dependent kinase 12 |
| CDK1 | Cyclin-dependent kinase 1 |
| AKT3 | RAC-gamma serine/threonine-protein kinase |
| CDK11B | Cyclin-dependent kinase 11B |
| PAK4 | Serine/threonine-protein kinase PAK 4 |
| MAPKAPK2 | MAP kinase-activated protein kinase 2 |
| TNK2 | Activated CDC42 kinase 1 |
| SIRT2 | NAD-dependent protein deacetylase sirtuin-2 |
| DAPK1 | Death-associated protein kinase 1 |
| ABL2 | Tyrosine-protein kinase ABL2 |
| PRKAA2 | 5′-AMP-activated protein kinase catalytic subunit alpha-2 |
| KAT2A | Histone acetyltransferase KAT2A |
| PBRM1 | Protein polybromo-1 |
| EIF2AK2 | Interferon-induced, double-stranded RNA-activated protein kinase |
| MAP3K7 | Mitogen-activated protein kinase kinase kinase 7 |
| MAPT | Microtubule-associated protein tau |
| RIPK1 | Receptor-interacting serine/threonine-protein kinase 1 |
| IRAK1 | Interleukin-1 receptor-associated kinase 1 |
| MAP4K1 | Mitogen-activated protein kinase kinase kinase kinase 1 |
| MARK4 | MAP/microtubule affinity-regulating kinase 4 |
| BRD9 | Bromodomain-containing protein 9 |
| RIPK2 | Receptor-interacting serine/threonine-protein kinase 2 |
| LIMK1 | LIM domain kinase 1 |
| STK38 | Serine/threonine-protein kinase 38 |
| TRIM24 | Transcription intermediary factor 1-alpha |
| SMARCA4 | Transcription activator BRG1 |
| PRKAA1 | 5′-AMP-activated protein kinase catalytic subunit alpha-1 |
| TBK1 | Serine/threonine-protein kinase TBK1 |
| KRAS | GTPase KRas |
| SMARCA2 | Probable global transcription activator SNF2L2 |
| PCNA | Proliferating cell nuclear antigen |
| BRD7 | Bromodomain-containing protein 7 |
| SUZ12 | Polycomb protein SUZ12 |
| IKZF1 | DNA-binding protein Ikaros |
| HTT | Huntingtin |
| SNCA | Alpha-synuclein |
| SLC9A1 | Sodium/hydrogen exchanger 1 |
| FER | Tyrosine-protein kinase Fer |
| MAP4K2 | Mitogen-activated protein kinase kinase kinase kinase 2 |
| DLG4 | Disks large homolog 4 |
| IKZF3 | Zinc finger protein Aiolos |
| SMAD3 | Mothers against decapentaplegic homolog 3 |
| PDE4A | cAMP-specific 3′,5′-cyclic phosphodiesterase 4A |
| HMGCR | 3-hydroxy-3-methylglutaryl-coenzyme A reductase |
| ABL1 | Tyrosine-protein kinase ABL1 |
| RARA | Retinoic acid receptor alpha |
| FLT3 | Receptor-type tyrosine-protein kinase FLT3 |
| RARG | Retinoic acid receptor gamma |
| FLT1 | Vascular endothelial growth factor receptor 1 |
| EZH2 | Histone-lysine N-methyltransferase EZH2 |
| EPHA1 | Ephrin type-A receptor 1 |
| AURKA | Aurora kinase A |
| CHEK1 | Serine/threonine-protein kinase Chk1 |
| WEE1 | Wee1-like protein kinase |
| PLK1 | Serine/threonine-protein kinase PLK1 |
| CDC7 | Cell division cycle 7-related protein kinase |
| AURKB | Aurora kinase B |
| PLK4 | Serine/threonine-protein kinase PLK4 |
| MDM2 | E3 ubiquitin-protein ligase Mdm2 |
| MAPK6 | Mitogen-activated protein kinase 6 |
| BUB1 | Mitotic checkpoint serine/threonine-protein kinase BUB1 |
| ESRRA | Steroid hormone receptor ERR1 |
| BCL6 | B-cell lymphoma 6 protein |
| MAPK12 | Mitogen-activated protein kinase 12 |
| CRABP2 | Cellular retinoic acid-binding protein 2 |
| HIPK1 | Homeodomain-interacting protein kinase 1 |
| ADRM1 | Proteasomal ubiquitin receptor ADRM1 |
| CDC20 | Cell division cycle protein 20 homolog |
| BUB1B | Mitotic checkpoint serine/threonine-protein kinase BUB1 beta |
| NTRK2 | BDNF/NT-3 growth factors receptor |
| NTRK1 | High affinity nerve growth factor receptor |
| CD274 | Programmed cell death 1 ligand 1 |
| NUAK1 | NUAK family SNF1-like kinase 1 |
| PDCD1 | Programmed cell death protein 1 |
| ERBB2 | Receptor tyrosine-protein kinase erbB-2 |
| EPHA3 | Ephrin type-A receptor 3 |
| PIK3CG | Phosphatidylinositol 4,5-bisphosphate 3-kinase catalytic subunit gamma isoform |
| MAP2K1 | Dual specificity mitogen-activated protein kinase kinase 1 |
| LCK | Tyrosine-protein kinase Lck |
| MAP2K2 | Dual specificity mitogen-activated protein kinase kinase 2 |
| PDE6D | Retinal rod rhodopsin-sensitive cGMP 3′,5′-cyclic phosphodiesterase subunit delta |
| JAK3 | Tyrosine-protein kinase JAK3 |
| GSK3B | Glycogen synthase kinase-3 beta |
| JAK2 | Tyrosine-protein kinase JAK2 |
| PRKCI | Protein kinase C iota type |
| FKBP1A | Peptidyl-prolyl cis-trans isomerase FKBP1A |
| DHODH | Dihydroorotate dehydrogenase |
| PIK3CA | Phosphatidylinositol 4,5-bisphosphate 3-kinase catalytic subunit alpha isoform |
| CDK6 | Cyclin-dependent kinase 6 |
| CDK4 | Cyclin-dependent kinase 4 |
| PDE4B | cAMP-specific 3′,5′-cyclic phosphodiesterase 4B |
| FYN | Tyrosine-protein kinase Fyn |
| TYK2 | Non-receptor tyrosine-protein kinase TYK2 |
| PDK1 | [Pyruvate dehydrogenase |
| PDK2 | [Pyruvate dehydrogenase |
| PDK3 | [Pyruvate dehydrogenase |
| YES1 | Tyrosine-protein kinase Yes |
| GSK3A | Glycogen synthase kinase-3 alpha |
| CDK16 | Cyclin-dependent kinase 16 |
| CSNK2A1 | Casein kinase II subunit alpha |
| CDK7 | Cyclin-dependent kinase 7 |
| PTK2B | Protein-tyrosine kinase 2-beta |
| MAPK10 | Mitogen-activated protein kinase 10 |
| MAPK9 | Mitogen-activated protein kinase 9 |
| MAPK8 | Mitogen-activated protein kinase 8 |
| CDK5 | Cyclin-dependent-like kinase 5 |
| METAP2 | Methionine aminopeptidase 2 |
| BRD2 | Bromodomain-containing protein 2 |
| MAPK3 | Mitogen-activated protein kinase 3 |
| TEC | Tyrosine-protein kinase Tec |
| CDK14 | Cyclin-dependent kinase 14 |
| CDK17 | Cyclin-dependent kinase 17 |
| MAP3K11 | Mitogen-activated protein kinase kinase kinase 11 |
| RPS6KB1 | Ribosomal protein S6 kinase beta-1 |
| CSK | Tyrosine-protein kinase CSK |
| MERTK | Tyrosine-protein kinase Mer |
| STK17B | Serine/threonine-protein kinase 17B |
| CSNK2A2 | Casein kinase II subunit alpha′ |
| RPS6KA1 | Ribosomal protein S6 kinase alpha-1 |
| MAPK13 | Mitogen-activated protein kinase 13 |
| GAK | Cyclin-G-associated kinase |
| CLK1 | Dual specificity protein kinase CLK1 |
| STK4 | Serine/threonine-protein kinase 4 |
| EIF4E | Eukaryotic translation initiation factor 4E |
| STK10 | Serine/threonine-protein kinase 10 |
| LRRK2 | Leucine-rich repeat serine/threonine-protein kinase 2 |
| TAOK3 | Serine/threonine-protein kinase TAO3 |
| MARK2 | Serine/threonine-protein kinase MARK2 |
| CSNK1D | Casein kinase I isoform delta |
| AAK1 | AP2-associated protein kinase 1 |
| IRAK3 | Interleukin-1 receptor-associated kinase 3 |
| STAT3 | Signal transducer and activator of transcription 3 |
| CAMKK1 | Calcium/calmodulin-dependent protein kinase kinase 1 |
| EED | Polycomb protein EED |
| CSNK1A1 | Casein kinase I isoform alpha |
| NEK1 | Serine/threonine-protein kinase Nek1 |
| BMP2K | BMP-2-inducible protein kinase |
| MAPK7 | Mitogen-activated protein kinase 7 |
| ULK1 | Serine/threonine-protein kinase ULK1 |
| RPS6KA3 | Ribosomal protein S6 kinase alpha-3 |
| PTPN11 | Tyrosine-protein phosphatase non-receptor type 11 |
| LIMK2 | LIM domain kinase 2 |
| CSNK1E | Casein kinase I isoform epsilon |
| EIF2AK4 | eIF-2-alpha kinase GCN2 |
| MAP2K5 | Dual specificity mitogen-activated protein kinase kinase 5 |
| MAP4K3 | Mitogen-activated protein kinase kinase kinase kinase 3 |
| VHL | von Hippel-Lindau disease tumor suppressor |
| MARK3 | MAP/microtubule affinity-regulating kinase 3 |
| TAOK2 | Serine/threonine-protein kinase TAO2 |
| MAP4K5 | Mitogen-activated protein kinase kinase kinase kinase 5 |
| SNRK | SNF-related serine/threonine-protein kinase |
| EEF2K | Eukaryotic elongation factor 2 kinase |
| SGK3 | Serine/threonine-protein kinase Sgk3 |
| AHR | Aryl hydrocarbon receptor |
| NEK4 | Serine/threonine-protein kinase Nek4 |
| NEK9 | Serine/threonine-protein kinase Nek9 |
| CIT | Citron Rho-interacting kinase |
| LATS1 | Serine/threonine-protein kinase LATS1 |
| MINK1 | Misshapen-like kinase 1 |
| SIK3 | Serine/threonine-protein kinase SIK3 |
| RPS6KA4 | Ribosomal protein S6 kinase alpha-4 |
| NEK3 | Serine/threonine-protein kinase Nek3 |
| SIK2 | Serine/threonine-protein kinase SIK2 |
| MAST3 | Microtubule-associated serine/threonine-protein kinase 3 |
| STK32C | Serine/threonine-protein kinase 32C |
| ALK | ALK tyrosine kinase receptor |
| EPHB4 | Ephrin type-B receptor 4 |
| PARP2 | Poly [ADP-ribose] polymerase 2 |
| PTK6 | Protein-tyrosine kinase 6 |
| PARP3 | Protein mono-ADP-ribosyltransferase PARP3 |
| CDK2 | Cyclin-dependent kinase 2 |
| CDK8 | Cyclin-dependent kinase 8 |
| BRDT | Bromodomain testis-specific protein |
| MAPKAPK5 | MAP kinase-activated protein kinase 5 |
| MAP3K1 | Mitogen-activated protein kinase kinase kinase 1 |
| CDK18 | Cyclin-dependent kinase 18 |
| CDK10 | Cyclin-dependent kinase 10 |
| TTK | Dual specificity protein kinase TTK |
| PIM2 | Serine/threonine-protein kinase pim-2 |
| PKMYT1 | Membrane-associated tyrosine- and threonine-specific cdc2-inhibitory kinase |
| MKNK2 | MAP kinase-interacting serine/threonine-protein kinase 2 |
| KAT2B | Histone acetyltransferase KAT2B |
| NEK2 | Serine/threonine-protein kinase Nek2 |
| HASPIN | Serine/threonine-protein kinase haspin |
| PIR | Pirin |
| CYP1B1 | Cytochrome P450 1B1 |
| ERN1 | Serine/threonine-protein kinase/endoribonuclease IRE1 |
| MELK | Maternal embryonic leucine zipper kinase |
| COQ8A | Atypical kinase COQ8A, mitochondrial |
| RIOK2 | Serine/threonine-protein kinase RIO2 |
| RPS6KA6 | Ribosomal protein S6 kinase alpha-6 |
| MAP3K20 | Mitogen-activated protein kinase kinase kinase 20 |
| MAPKAPK3 | MAP kinase-activated protein kinase 3 |
| ULK3 | Serine/threonine-protein kinase ULK3 |
| MAP3K21 | Mitogen-activated protein kinase kinase kinase 21 |
| COQ8B | Atypical kinase COQ8B, mitochondrial |
| TNK1 | Non-receptor tyrosine-protein kinase TNK1 |
| BMPR1A | Bone morphogenetic protein receptor type-1A |
| STK17A | Serine/threonine-protein kinase 17A |
| CDK11A | Cyclin-dependent kinase 11A |
| TESK2 | Dual specificity testis-specific protein kinase 2 |
| NLK | Serine/threonine-protein kinase NLK |
| STK35 | Serine/threonine-protein kinase 35 |
| PKN3 | Serine/threonine-protein kinase N3 |
| STK33 | Serine/threonine-protein kinase 33 |
| SBK1 | Serine/threonine-protein kinase SBK1 |
| SLC9A2 | Sodium/hydrogen exchanger 2 |
| CSNK2A3 | Casein kinase II subunit alpha 3 |
| TACC3 | Transforming acidic coiled-coil-containing protein 3 |
| GSPT1 | Eukaryotic peptide chain release factor GTP-binding subunit ERF3A |
| SLC9A7 | Sodium/hydrogen exchanger 7 |
| RPS6KC1 | Ribosomal protein S6 kinase delta-1 |
| TBCK | TBC domain-containing protein kinase-like protein |
| CRABP1 | Cellular retinoic acid-binding protein 1 |
| BCKDK | [3-methyl-2-oxobutanoate dehydrogenase [lipoamide]] kinase, mitochondrial |
| TRPM7 | Transient receptor potential cation channel subfamily M member 7 |
| TRIB3 | Tribbles homolog 3 |
| UHMK1 | Serine/threonine-protein kinase Kist |
| PDIK1L | Serine/threonine-protein kinase PDIK1L |
| STK40 | Serine/threonine-protein kinase 40 |
| SLC9A4 | Sodium/hydrogen exchanger 4 |
| EPHB3 | Ephrin type-B receptor 3 |
| NTRK3 | NT-3 growth factor receptor |
| MAP3K12 | Mitogen-activated protein kinase kinase kinase 12 |
| MAPK11 | Mitogen-activated protein kinase 11 |
| DDR2 | Discoidin domain-containing receptor 2 |
| RARB | Retinoic acid receptor beta |
| PDE4C | cAMP-specific 3′,5′-cyclic phosphodiesterase 4C |
| IDO1 | Indoleamine 2,3-dioxygenase 1 |
| SLC9B1 | Sodium/hydrogen exchanger 9B1 |
| PRAG1 | Inactive tyrosine-protein kinase PRAG1 |

5. The engineered platelet of claim 1, wherein the POI is an extracellular protein selected from the following table:

| | |
|---|---|
| HER2 | Human epidermal growth factor receptor 2 |
| EGFR | Epidermal growth factor receptor |
| HGFR | Hepatocyte growth factor receptor |
| PTK-7 | Protein Tyrosine Kinase 7 |
| CD71 (TfR1) | Cluster of Differentiation 71; Transferrin |
| | receptor protein 1 |
| ApoE4 | Apolipoprotein E4 |
| PD-L1 | Programmed death ligand 1 |

6. The engineered platelet of claim 1, wherein the POI ligand is covalently linked to the heat HSP90 by a chemical linker.

7. The engineered platelet of claim 1, wherein the chemical linker is an N-acyl-N-alkyl sulfonamide (NASA) linker.

8. A method of making an engineered platelet, comprising
covalently linking a ligand for a POI to an HSP90 to form an to form an HSP90 anchoring chimera, and
incubating a suspension of platelet cells or platelet-derived microparticles with the HSP90 anchoring chimera to load the HSP-90 anchoring chimera in the platelet cells or platelet-derived microparticles and form the engineered platelets.

9. The method of claim 8, wherein the HSP90 chimera is prepared by bridging the POI ligand and an HSP90 ligand by a linker that reacts with a nucleophilic amino acid side chain to generate an HSP90-anchoring chimera, incubating the HSP90-anchoring chimera with HSP90 to transfer and tether the POI ligand to the HSP90, and releasing the HSP90 ligand from the HSP90 to provide the HSP90 chimera.

10. The method of claim 9, wherein the linker is an N-acyl-N-alkyl sulfonamide linker, an ortho-dibromophenyl benzoate linker, an electrophilic phenylsulfonate ester group, or an N-sulfonyl pyridine linker.

11. The method of claim 9, wherein the HSP90 ligand is the HSP90 N-terminal ATPase domain inhibitor PU-H71.

12. A method of degrading an intracellular POI via a ubiquitin-protease system, comprising contacting a disease site with the engineered platelet of claim 1, wherein the POI ligand binds the intracellular POI, and wherein the engineered platelet transfers the HSP-90 anchoring chimera to cells at the disease site, thereby binding to and degrading the intracellular POI.

13. The method of claim 12, wherein the intracellular POI is selected from the following table:

| | |
|---|---|
| ESR1 | Estrogen receptor |
| AR | Androgen receptor |
| BTK | Tyrosine-protein kinase BTK |
| IRAK4 | Interleukin-1 receptor-associated kinase 4 |
| EGFR | Epidermal growth factor receptor |
| MET | Hepatocyte growth factor receptor |
| KIT | Mast/stem cell growth factor receptor Kit |
| EPHA2 | Ephrin type-A receptor 2 |
| PDE4D | cAMP-specific 3′,5′-cyclic phosphodiesterase 4D |
| SRC | Proto-oncogene tyrosine-protein kinase Src |
| BRAF | Serine/threonine-protein kinase B-raf |
| FGFR2 | Fibroblast growth factor receptor 2 |
| FGFR1 | Fibroblast growth factor receptor 1 |
| LYN | Tyrosine-protein kinase Lyn |
| ITK | Tyrosine-protein kinase ITK/TSK |
| PARP1 | Poly [ADP-ribose] polymerase 1 |
| HDAC2 | Histone deacetylase 2 |
| HDAC3 | Histone deacetylase 3 |
| JAK1 | Tyrosine-protein kinase JAK1 |
| BCL2 | Apoptosis regulator Bcl-2 |
| HDAC6 | Histone deacetylase 6 |
| CRBN | Protein cereblon |
| EPHB2 | Ephrin type-B receptor 2 |
| BLK | Tyrosine-protein kinase Blk |
| HDAC1 | Histone deacetylase 1 |
| IGF1R | Insulin-like growth factor 1 receptor |
| TGFBR1 | TGF-beta receptor type-1 |
| AKT2 | RAC-beta serine/threonine-protein kinase |
| AKT1 | RAC-alpha serine/threonine-protein kinase |
| PTK2 | Focal adhesion kinase 1 |
| MAPK1 | Mitogen-activated protein kinase 1 |
| MAPK14 | Mitogen-activated protein kinase 14 |
| CDK9 | Cyclin-dependent kinase 9 |
| MCL1 | Induced myeloid leukemia cell differentiation protein Mcl-1 |
| BRD4 | Bromodomain-containing protein 4 |
| BRD3 | Bromodomain-containing protein 3 |
| CDK13 | Cyclin-dependent kinase 13 |
| BCL2L1 | Bcl-2-like protein 1 |
| CDK12 | Cyclin-dependent kinase 12 |
| CDK1 | Cyclin-dependent kinase 1 |
| AKT3 | RAC-gamma serine/threonine-protein kinase |
| CDK11B | Cyclin-dependent kinase 11B |
| PAK4 | Serine/threonine-protein kinase PAK 4 |
| MAPKAPK2 | MAP kinase-activated protein kinase 2 |
| TNK2 | Activated CDC42 kinase 1 |
| SIRT2 | NAD-dependent protein deacetylase sirtuin-2 |
| DAPK1 | Death-associated protein kinase 1 |
| ABL2 | Tyrosine-protein kinase ABL2 |
| PRKAA2 | 5′-AMP-activated protein kinase catalytic subunit alpha-2 |
| KAT2A | Histone acetyltransferase KAT2A |
| PBRM1 | Protein polybromo-1 |
| EIF2AK2 | Interferon-induced, double-stranded RNA-activated protein kinase |
| MAP3K7 | Mitogen-activated protein kinase kinase kinase 7 |
| MAPT | Microtubule-associated protein tau |
| RIPK1 | Receptor-interacting serine/threonine-protein kinase 1 |
| IRAK1 | Interleukin-1 receptor-associated kinase 1 |
| MAP4K1 | Mitogen-activated protein kinase kinase kinase kinase 1 |
| MARK4 | MAP/microtubule affinity-regulating kinase 4 |
| BRD9 | Bromodomain-containing protein 9 |
| RIPK2 | Receptor-interacting serine/threonine-protein kinase 2 |
| LIMK1 | LIM domain kinase 1 |
| STK38 | Serine/threonine-protein kinase 38 |
| TRIM24 | Transcription intermediary factor 1-alpha |
| SMARCA4 | Transcription activator BRG1 |
| PRKAA1 | 5′-AMP-activated protein kinase catalytic subunit alpha-1 |
| TBK1 | Serine/threonine-protein kinase TBK1 |
| KRAS | GTPase KRas |
| SMARCA2 | Probable global transcription activator SNF2L2 |
| PCNA | Proliferating cell nuclear antigen |
| BRD7 | Bromodomain-containing protein 7 |
| SUZ12 | Polycomb protein SUZ12 |
| IKZF1 | DNA-binding protein Ikaros |
| HTT | Huntingtin |
| SNCA | Alpha-synuclein |
| SLC9A1 | Sodium/hydrogen exchanger 1 |
| FER | Tyrosine-protein kinase Fer |
| MAP4K2 | Mitogen-activated protein kinase kinase kinase kinase 2 |
| DLG4 | Disks large homolog 4 |
| IKZF3 | Zinc finger protein Aiolos |
| SMAD3 | Mothers against decapentaplegic homolog 3 |
| PDE4A | cAMP-specific 3′,5′-cyclic phosphodiesterase 4A |
| HMGCR | 3-hydroxy-3-methylglutaryl-coenzyme A reductase |
| ABL1 | Tyrosine-protein kinase ABL1 |
| RARA | Retinoic acid receptor alpha |
| FLT3 | Receptor-type tyrosine-protein kinase FLT3 |
| RARG | Retinoic acid receptor gamma |
| FLT1 | Vascular endothelial growth factor receptor 1 |
| EZH2 | Histone-lysine N-methyltransferase EZH2 |
| EPHA1 | Ephrin type-A receptor 1 |
| AURKA | Aurora kinase A |
| CHEK1 | Serine/threonine-protein kinase Chk1 |
| WEE1 | Wee 1-like protein kinase |
| PLK1 | Serine/threonine-protein kinase PLK1 |
| CDC7 | Cell division cycle 7-related protein kinase |
| AURKB | Aurora kinase B |
| PLK4 | Serine/threonine-protein kinase PLK4 |
| MDM2 | E3 ubiquitin-protein ligase Mdm2 |
| MAPK6 | Mitogen-activated protein kinase 6 |
| BUB1 | Mitotic checkpoint serine/threonine-protein kinase BUB1 |
| ESRRA | Steroid hormone receptor ERR1 |
| BCL6 | B-cell lymphoma 6 protein |
| MAPK12 | Mitogen-activated protein kinase 12 |
| CRABP2 | Cellular retinoic acid-binding protein 2 |
| HIPK1 | Homeodomain-interacting protein kinase 1 |
| ADRM1 | Proteasomal ubiquitin receptor ADRM1 |
| CDC20 | Cell division cycle protein 20 homolog |
| BUB1B | Mitotic checkpoint serine/threonine-protein kinase BUB1 beta |
| NTRK2 | BDNF/NT-3 growth factors receptor |
| NTRK1 | High affinity nerve growth factor receptor |
| CD274 | Programmed cell death 1 ligand 1 |
| NUAK1 | NUAK family SNF1-like kinase 1 |
| PDCD1 | Programmed cell death protein 1 |
| ERBB2 | Receptor tyrosine-protein kinase erbB-2 |
| EPHA3 | Ephrin type-A receptor 3 |
| PIK3CG | Phosphatidylinositol 4,5-bisphosphate 3-kinase catalytic subunit gamma isoform |
| MAP2K1 | Dual specificity mitogen-activated protein kinase kinase 1 |
| LCK | Tyrosine-protein kinase Lck |
| MAP2K2 | Dual specificity mitogen-activated protein kinase kinase 2 |
| PDE6D | Retinal rod rhodopsin-sensitive cGMP 3′,5′-cyclic phosphodiesterase subunit delta |
| JAK3 | Tyrosine-protein kinase JAK3 |
| GSK3B | Glycogen synthase kinase-3 beta |
| JAK2 | Tyrosine-protein kinase JAK2 |
| PRKCI | Protein kinase C iota type |
| FKBP1A | Peptidyl-prolyl cis-trans isomerase FKBP1A |
| DHODH | Dihydroorotate dehydrogenase |
| PIK3CA | Phosphatidylinositol 4,5-bisphosphate 3-kinase catalytic subunit alpha isoform |
| CDK6 | Cyclin-dependent kinase 6 |
| CDK4 | Cyclin-dependent kinase 4 |
| PDE4B | cAMP-specific 3′,5′-cyclic phosphodiesterase 4B |
| FYN | Tyrosine-protein kinase Fyn |
| TYK2 | Non-receptor tyrosine-protein kinase TYK2 |
| PDK1 | [Pyruvate dehydrogenase |
| PDK2 | [Pyruvate dehydrogenase |
| PDK3 | [Pyruvate dehydrogenase |
| YES1 | Tyrosine-protein kinase Yes |
| GSK3A | Glycogen synthase kinase-3 alpha |
| CDK16 | Cyclin-dependent kinase 16 |
| CSNK2A1 | Casein kinase II subunit alpha |
| CDK7 | Cyclin-dependent kinase 7 |
| PTK2B | Protein-tyrosine kinase 2-beta |
| MAPK10 | Mitogen-activated protein kinase 10 |
| MAPK9 | Mitogen-activated protein kinase 9 |
| MAPK8 | Mitogen-activated protein kinase 8 |
| CDK5 | Cyclin-dependent-like kinase 5 |
| METAP2 | Methionine aminopeptidase 2 |
| BRD2 | Bromodomain-containing protein 2 |
| MAPK3 | Mitogen-activated protein kinase 3 |
| TEC | Tyrosine-protein kinase Tec |
| CDK14 | Cyclin-dependent kinase 14 |
| CDK17 | Cyclin-dependent kinase 17 |
| MAP3K11 | Mitogen-activated protein kinase kinase kinase 11 |
| RPS6KB1 | Ribosomal protein S6 kinase beta-1 |
| CSK | Tyrosine-protein kinase CSK |
| MERTK | Tyrosine-protein kinase Mer |
| STK17B | Serine/threonine-protein kinase 17B |
| CSNK2A2 | Casein kinase II subunit alpha′ |
| RPS6KA1 | Ribosomal protein S6 kinase alpha-1 |
| MAPK13 | Mitogen-activated protein kinase 13 |
| GAK | Cyclin-G-associated kinase |
| CLK1 | Dual specificity protein kinase CLK1 |
| STK4 | Serine/threonine-protein kinase 4 |
| EIF4E | Eukaryotic translation initiation factor 4E |
| STK10 | Serine/threonine-protein kinase 10 |
| LRRK2 | Leucine-rich repeat serine/threonine-protein kinase 2 |
| TAOK3 | Serine/threonine-protein kinase TAO3 |
| MARK2 | Serine/threonine-protein kinase MARK2 |
| CSNK1D | Casein kinase I isoform delta |
| AAK1 | AP2-associated protein kinase 1 |
| IRAK3 | Interleukin-1 receptor-associated kinase 3 |
| STAT3 | Signal transducer and activator of transcription 3 |
| CAMKK1 | Calcium/calmodulin-dependent protein kinase kinase 1 |
| EED | Polycomb protein EED |
| CSNK1A1 | Casein kinase I isoform alpha |
| NEK1 | Serine/threonine-protein kinase Nek1 |
| BMP2K | BMP-2-inducible protein kinase |
| MAPK7 | Mitogen-activated protein kinase 7 |
| ULK1 | Serine/threonine-protein kinase ULK1 |
| RPS6KA3 | Ribosomal protein S6 kinase alpha-3 |
| PTPN11 | Tyrosine-protein phosphatase non-receptor type 11 |
| LIMK2 | LIM domain kinase 2 |
| CSNK1E | Casein kinase I isoform epsilon |
| EIF2AK4 | eIF-2-alpha kinase GCN2 |
| MAP2K5 | Dual specificity mitogen-activated protein kinase kinase 5 |
| MAP4K3 | Mitogen-activated protein kinase kinase kinase kinase 3 |
| VHL | von Hippel-Lindau disease tumor suppressor |
| MARK3 | MAP/microtubule affinity-regulating kinase 3 |
| TAOK2 | Serine/threonine-protein kinase TAO2 |
| MAP4K5 | Mitogen-activated protein kinase kinase kinase kinase 5 |
| SNRK | SNF-related serine/threonine-protein kinase |
| EEF2K | Eukaryotic elongation factor 2 kinase |
| SGK3 | Serine/threonine-protein kinase Sgk3 |
| AHR | Aryl hydrocarbon receptor |
| NEK4 | Serine/threonine-protein kinase Nek4 |
| NEK9 | Serine/threonine-protein kinase Nek9 |
| CIT | Citron Rho-interacting kinase |
| LATS1 | Serine/threonine-protein kinase LATS1 |
| MINK1 | Misshapen-like kinase 1 |
| SIK3 | Serine/threonine-protein kinase SIK3 |
| RPS6KA4 | Ribosomal protein S6 kinase alpha-4 |
| NEK3 | Serine/threonine-protein kinase Nek3 |
| SIK2 | Serine/threonine-protein kinase SIK2 |
| MAST3 | Microtubule-associated serine/threonine-protein kinase 3 |
| STK32C | Serine/threonine-protein kinase 32C |
| ALK | ALK tyrosine kinase receptor |
| EPHB4 | Ephrin type-B receptor 4 |
| PARP2 | Poly [ADP-ribose] polymerase 2 |
| PTK6 | Protein-tyrosine kinase 6 |
| PARP3 | Protein mono-ADP-ribosyltransferase PARP3 |
| CDK2 | Cyclin-dependent kinase 2 |
| CDK8 | Cyclin-dependent kinase 8 |
| BRDT | Bromodomain testis-specific protein |
| MAPKAPK5 | MAP kinase-activated protein kinase 5 |
| MAP3K1 | Mitogen-activated protein kinase kinase kinase 1 |
| CDK18 | Cyclin-dependent kinase 18 |
| CDK10 | Cyclin-dependent kinase 10 |
| TTK | Dual specificity protein kinase TTK |
| PIM2 | Serine/threonine-protein kinase pim-2 |
| PKMYT1 | Membrane-associated tyrosine- and threonine-specific cdc2-inhibitory kinase |
| MKNK2 | MAP kinase-interacting serine/threonine-protein kinase 2 |
| KAT2B | Histone acetyltransferase KAT2B |
| NEK2 | Serine/threonine-protein kinase Nek2 |
| HASPIN | Serine/threonine-protein kinase haspin |
| PIR | Pirin |
| CYP1B1 | Cytochrome P450 1B1 |
| ERN1 | Serine/threonine-protein kinase/endoribonuclease IRE1 |
| MELK | Maternal embryonic leucine zipper kinase |
| COQ8A | Atypical kinase COQ8A, mitochondrial |
| RIOK2 | Serine/threonine-protein kinase RIO2 |
| RPS6KA6 | Ribosomal protein S6 kinase alpha-6 |
| MAP3K20 | Mitogen-activated protein kinase kinase kinase 20 |
| MAPKAPK3 | MAP kinase-activated protein kinase 3 |
| ULK3 | Serine/threonine-protein kinase ULK3 |
| MAP3K21 | Mitogen-activated protein kinase kinase kinase 21 |
| COQ8B | Atypical kinase COQ8B, mitochondrial |
| TNK1 | Non-receptor tyrosine-protein kinase TNK1 |
| BMPR1A | Bone morphogenetic protein receptor type-1A |
| STK17A | Serine/threonine-protein kinase 17A |
| CDK11A | Cyclin-dependent kinase 11A |
| TESK2 | Dual specificity testis-specific protein kinase 2 |
| NLK | Serine/threonine-protein kinase NLK |
| STK35 | Serine/threonine-protein kinase 35 |
| PKN3 | Serine/threonine-protein kinase N3 |
| STK33 | Serine/threonine-protein kinase 33 |
| SBK1 | Serine/threonine-protein kinase SBK1 |
| SLC9A2 | Sodium/hydrogen exchanger 2 |
| CSNK2A3 | Casein kinase II subunit alpha 3 |
| TACC3 | Transforming acidic coiled-coil-containing protein 3 |
| GSPT1 | Eukaryotic peptide chain release factor GTP-binding subunit ERF3A |
| SLC9A7 | Sodium/hydrogen exchanger 7 |
| RPS6KC1 | Ribosomal protein S6 kinase delta-1 |
| TBCK | TBC domain-containing protein kinase-like protein |
| CRABP1 | Cellular retinoic acid-binding protein 1 |
| BCKDK | [3-methyl-2-oxobutanoate dehydrogenase [lipoamide]] kinase, mitochondrial |
| TRPM7 | Transient receptor potential cation channel subfamily M member 7 |
| TRIB3 | Tribbles homolog 3 |
| UHMK1 | Serine/threonine-protein kinase Kist |
| PDIK1L | Serine/threonine-protein kinase PDIK1L |
| STK40 | Serine/threonine-protein kinase 40 |
| SLC9A4 | Sodium/hydrogen exchanger 4 |
| EPHB3 | Ephrin type-B receptor 3 |
| NTRK3 | NT-3 growth factor receptor |
| MAP3K12 | Mitogen-activated protein kinase kinase kinase 12 |
| MAPK11 | Mitogen-activated protein kinase 11 |
| DDR2 | Discoidin domain-containing receptor 2 |
| RARB | Retinoic acid receptor beta |
| PDE4C | cAMP-specific 3′,5′-cyclic phosphodiesterase 4C |
| IDO1 | Indoleamine 2,3-dioxygenase 1 |
| SLC9B1 | Sodium/hydrogen exchanger 9B1 |
| PRAG1 | Inactive tyrosine-protein kinase PRAG1 |

14. The method of claim 12, wherein the disease site is in a patient suffering from a postoperative tumor or a wound-associated disease.

15. The method of claim 14, wherein the engineered platelets are administered by intravenous injection or locoregional administration.

16. A method of degrading an extracellular POI via a lysosome-associated pathway, comprising contacting a disease site with the engineered platelet of claim 1, wherein the POI ligand binds the extracellular POI, and wherein the engineered platelet releases the HSP90 anchoring chimera to the extracellular space at the disease site, thereby binding to the extracellular POI and guiding it to the lysosome for degradation.

17. The method of claim 16, wherein the POI is an extracellular protein selected from the following table:

| | |
|---|---|
| HER2 | Human epidermal growth factor receptor 2 |
| EGFR | Epidermal growth factor receptor |
| HGFR | Hepatocyte growth factor receptor |
| PTK-7 | Protein Tyrosine Kinase 7 |
| CD71 (TfR1) | Cluster of Differentiation 71; Transferrin |
| | receptor protein 1 |
| ApoE4 | Apolipoprotein E4 |
| PD-L1 | Programmed death ligand 1 |

18. The method of claim 16, wherein the disease site is in a patient suffering from a postoperative tumor or a wound-associated diseases.

19. The method of claim 16, wherein the engineered platelets are administered by intravenous injection or locoregional administration.
